(19) United States
(12) Patent Application Publication
(10) Pub. No.: US 2026/0137940 A1
(43) Pub. Date: May 21, 2026

(54) SENSORY MEDICAL DEVICE WITH EXPANDED FEATURES

(71) Applicants: COCHLEAR LIMITED, Macquarie University, NSW (AU); UNIVERSIDAD DE LAS PALMAS DE GRAN CANARIA, Las Palmas de Gran Canaria (ES)

(72) Inventors: Koen Erik VAN DEN HEUVEL, Macquarie University (AU); Erika J. VAN BAELEN, Macquarie University (AU); Angel Manuel RAMOS MACIAS, Las Palmas de Gran Canaria (ES); Carl VAN HIMBEECK, Zottegem (BE); Angel RAMOS DE MIGUEL, Las Palmas de Gran Canaria (ES); Kenneth OPLINGER, Macquarie University (AU)

(21) Appl. No.: 19/121,584

(22) PCT Filed: Sep. 14, 2023
(86) PCT No.: PCT/IB2023/059122

§ 371 Date: Apr. 16, 2025

(30) Foreign Application Priority Data
Oct. 17, 2022 (EP) 22382991.2
Aug. 30, 2023 (EP) 23382883.9

(51) Int. Cl. A61N 1/36; A61N 1/372

(52) U.S. Cl. CPC A61N 1/36067; A61N 1/36038; A61N 1/36064; A61N 1/36128; A61N 1/37217

(57) ABSTRACT

A system, including a first subsystem configured to neurologically affect a human when activated and a second subsystem configured to provide an indication that the system is activated and/or not activated, wherein the system is a sensory management and/or sensory stimulation system.

### FUNDING STATEMENT

The project leading to this application has received funding from the European Union's Horizon 2020 research and innovation programme under grant agreement No 801127.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an exemplary embodiment, there is a system, comprising a first subsystem configured to neurologically affect a human when activated and a second subsystem configured to obtain data based on an ambient environment of the system, wherein the system is configured to control the first subsystem, at least in part, based on the obtained data, and the system is a human balance medical system.

In an exemplary embodiment, there is a method, comprising automatically obtaining data based on a changeable environment of a balance-impaired human and controlling, at least partially, input to the brain of the human from a vestibula system of the human based on the obtained data.

In an exemplary embodiment, there is a method, comprising obtaining data based on an ambient environment of a balance-impaired human, varying the ambient environment based on the obtained data, operating a balance sensory medical device connected to the human based on the varied ambient environment.

In an exemplary embodiment, there is an apparatus, comprising one or more electrodes, a power source, a light capture device and a control unit, wherein the apparatus is configured so that the control unit controls electrical signal(s) to the one or more electrodes to provide balance therapy to a recipient of the apparatus, the apparatus also configured so that the control unit controls the electrical signal(s) based on output from the light capture device.

In an exemplary embodiment, there is a human balance medical system, comprising a neurological stimulator subsystem configured to influence neurological signals to a brain of a recipient of the human balance medical system to improve balance of the recipient and a power source, wherein the neurological stimulatory subsystem is powered by the power source, and the human balance medical system is a smart human balance medical system. in an embodiment, there is a prosthetic human balance medical device, comprising:
at least one of:
   (1) one or more external electrodes; or
   (2) one or more implantable electrodes configured to be exposed to body fluids for at least 12 months;
a battery, wherein the battery is rechargeable or disposable;
a control circuit configured to controls electrical signal(s) to the one or more electrodes to provide balance therapy to a recipient of the prosthetic medical device; and
a control circuit configured to provide output from the device indicative of an activation state and/or an efficacy of the device.

In an exemplary embodiment, there is a prosthetic human balance medical device, comprising:
at least one of:
   (1) one or more external electrodes; or
   (2) one or more implantable electrodes configured to be exposed to body fluids for at least 12 months;
a battery, wherein the battery is rechargeable or disposable;
a photoelectric device configured to capture and/or be sensitive to ambient light, wherein the photoelectric device includes one or more of photoactive region(s), photoresistor(s), photodiode(s), photodetector(s), phototransistor(s) or charged coupled device; and
a control circuit configured to controls electrical signal(s) to the one or more electrodes to provide balance therapy to a recipient of the prosthetic medical device, and configured to control electrical signal(s) based on output from the photoelectric device to improve balance of a recipient of the prosthetic medical device.

In an exemplary embodiment, there is a system, comprising a first subsystem configured to neurologically affect a human when activated and a second subsystem configured to provide an indication that the system is activated and/or not activated, wherein the system is a sensory management and/or sensory stimulation system.

In an exemplary embodiment, there is a non-transitory computer-readable media having recorded thereon, a computer program for executing at least a portion of a method, the computer program including code for automatically determining whether a system is functioning and/or is not functioning and/or how well the system is functioning and/or if the system is capable of functioning, wherein the system is a sensory management and/or sensory stimulation system and code for providing an indication that the system is functioning and/or is not functioning and/or how well the system is functioning and/or if the system is capable of functioning and/or how well the system will function.

In an exemplary embodiment, there is a method, comprising operating a medical device connected to a human, wherein the medical device is configured to stimulate the inner ear of the human and prior to, during and/or after the action of operating, automatically evaluating efficacy of the medical device.

### BRIEF DESCRIPTION OF THE DRAWINGS

Embodiments are described below with reference to the attached drawings, in which:

FIG. 1 is a perspective view of an exemplary hearing prosthesis;

FIG. 2 presents a functional block diagram of an exemplary cochlear implant;

FIG. 3A and FIGS. 3B and 3C present exemplary systems of communication between devices;

FIG. 4 presents an exemplary retinal prosthesis;

FIG. 5 presents an exemplary vestibular implant;

FIGS. 6 and 7 provide details of another exemplary vestibular implant;

FIG. 8 presents an exemplary external component;

FIGS. 9-12 present exemplary flowcharts for exemplary methods;

FIG. 13 presents an exemplary implant;

FIGS. 14-17 and 22-26 and 29 present exemplary flowcharts for exemplary methods;

FIGS. 18 and 19 present anatomical structures;

FIG. 20 presents another exemplary vestibular implant; and

FIG. 21 presents electrode insertions points for an exemplary embodiment;

FIGS. 27 and 28 present exemplary functional block diagrams of an exemplary system;

FIGS. 30 and 31 present exemplary sensory affecting systems.

### DETAILED DESCRIPTION

Merely for ease of description, the techniques presented herein are described herein with reference by way of background to an illustrative medical device, namely a cochlear implant and/or This is because in some embodiments, but not all, features of such an implant have general and/or specific applicability to a vestibular implant, which in turn has genera/and/or specific applicability to a balance prosthesis according to the teachings herein. However, it is noted that the techniques presented herein can also be used with a variety of other medical devices that, while providing a wide range of therapeutic benefits to recipients, patients, or other users, may benefit from setting changes based on the location of the medical device. In this regard, for example, as just noted, the techniques presented herein may be used to with a vestibular implant and/or a balance medical device (such as a balance prosthesis), and/or a retinal implant, with respect to a particular human being. And with regard to the latter, the techniques presented herein are also described with reference by way of background to another illustrative medical device, namely a retinal implant. But to be clear, the techniques presented herein are squarely applicable to the technology of balance medical devices, vestibular devices (e.g., vestibular implants), where the two are not mutually exclusive. Embodiments can be also directed to visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc.

And briefly, in an embodiment, a commercially available cochlear implant external component (a so-called sound processor) can be modified to implement at least some of the teachings herein vis-à-vis vestibular stimulation (e.g., by changing the software and/or firmware, and possibly changing a chip or some circuitry, but starting with an external cochlear implant component). The implant can be a modified implantable portion of a cochlear implant, for that matter. Note that these changes change the initial device from a cochlear implant to a vestibular implant.

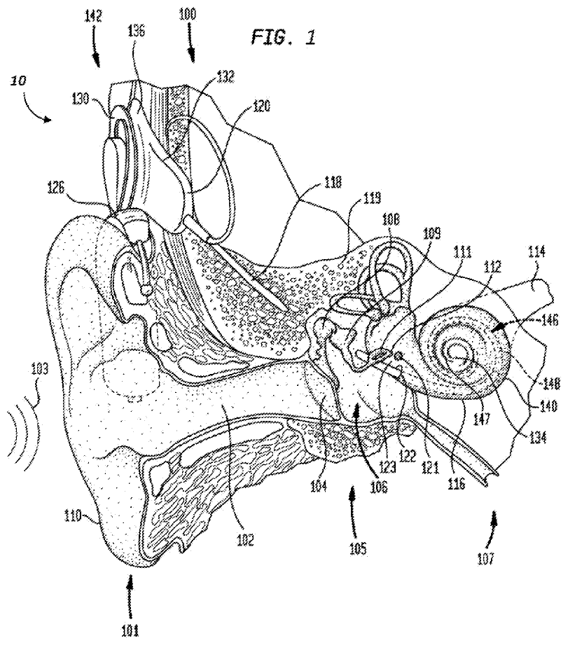

Again, as will be noted below, some embodiments of the vestibular implant use at least some features of a cochlear implant. And thus, we first describe a cochlear implant. FIG. 1 is a perspective view of a cochlear implant, referred to as cochlear implant 100, implanted in a recipient, to which some embodiments detailed herein and/or variations thereof are applicable. Particularly, as will be detailed below, there are aspects of a cochlear implant that are utilized with respect to a vestibular implant, and thus there is utility in describing features of the cochlear implant for purposes of understanding a vestibular implant. The cochlear implant 100 is part of a system 10 that can include external components in some embodiments, as will be detailed below. Additionally, it is noted that the teachings detailed herein are also applicable to other types of hearing prostheses, such as, by way of example only and not by way of limitation, bone conduction devices (percutaneous, active transcutaneous and/or passive transcutaneous), direct acoustic cochlear stimulators, middle ear implants, and conventional hearing aids, etc. Indeed, it is noted that the teachings detailed herein are also applicable to so-called multi-mode devices. In an exemplary embodiment, these multi-mode devices apply both electrical stimulation and acoustic stimulation to the recipient. In an exemplary embodiment, these multi-mode devices evoke a hearing percept via electrical hearing and bone conduction hearing.

In view of the above, at least some embodiments detailed herein and/or variations thereof are directed towards a body-worn sensory supplement medical device (e.g., a balance prosthesis and/or a vestibular prosthesis (more on this below), or the hearing prosthesis of FIG. 1, (or a device that uses at least one or more of the features of such)). A balance device can supplement the one or more of the senses that provide the sensation of balance.

Some embodiments include the application of the teachings detailed herein to any type of sensory supplement medical device to which the teachings detailed herein are enabled for use therein in a utilitarian manner. In this regard, the phrase sensory supplement medical device refers to any device that functions to provide sensation to a recipient irrespective of whether the applicable natural sense is only partially impaired or completely impaired, or indeed never existed.

Returning back to FIG. 1, the recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1 with an external device 142, that is part of system 10 (along with cochlear implant 100), which, as described below, is configured to provide power to the cochlear implant, where the implanted cochlear implant includes a battery that is recharged by the power provided from the external device 142.

In the illustrative arrangement of FIG. 1, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate electrode assembly 118. In some embodiments, internal energy transfer assembly 132 and main implantable component 120 are hermetically sealed within a biocompatible housing. In some embodiments, main implantable component 120 includes an implantable microphone assembly (not shown) and a sound processing unit (not shown) to convert the sound signals received by the implantable microphone in internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement.

Main implantable component 120 further includes a stimulator unit (also not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

Thus, as seen above, one variety of implanted devices depends on an external component to provide certain functionality and/or power. For example, the recipient of the implanted device can wear an external component that provides power and/or data (e.g., a signal representative of sound) to the implanted portion that allow the implanted device to function. In particular, the implanted device can lack a battery and can instead be totally dependent on an external power source providing continuous power for the implanted device to function. Although the external power source can continuously provide power, characteristics of the provided power need not be constant and may fluctuate. Additionally, where the implanted device is an auditory prosthesis such as a cochlear implant, the implanted device can lack its own sound input device (e.g., a microphone). It is sometimes utilitarian to remove the external component. For example, it is common for a recipient of an auditory prosthesis to remove an external portion of the prosthesis while sleeping. Doing so can result in loss of function of the implanted portion of the prosthesis, which can make it impossible for recipient to hear ambient sound. This can be less than utilitarian and can result in the recipient being unable to hear while sleeping. Loss of function would also prevent the implanted portion from responding to signals representative of streamed content (e.g., music streamed from a phone) or providing other functionality, such as providing tinnitus suppression noise.

The external component that provides power and/or data can be worn by the recipient, as detailed above. While a wearable external device is worn by a recipient, the external device is typically in very close proximity and tightly aligned with an implanted component. The wearable external device can be configured to operate in these conditions. Conversely, in some instances, an unworn device can generally be further away and less tightly aligned with the implanted component. This can create difficulties where the implanted device depends on an external device for power and data (e.g., where the implanted device lacks its own battery and microphone), and the external device can need to continuously and consistently provide power and data in order to allow for continuous and consistent functionality of the implanted device.

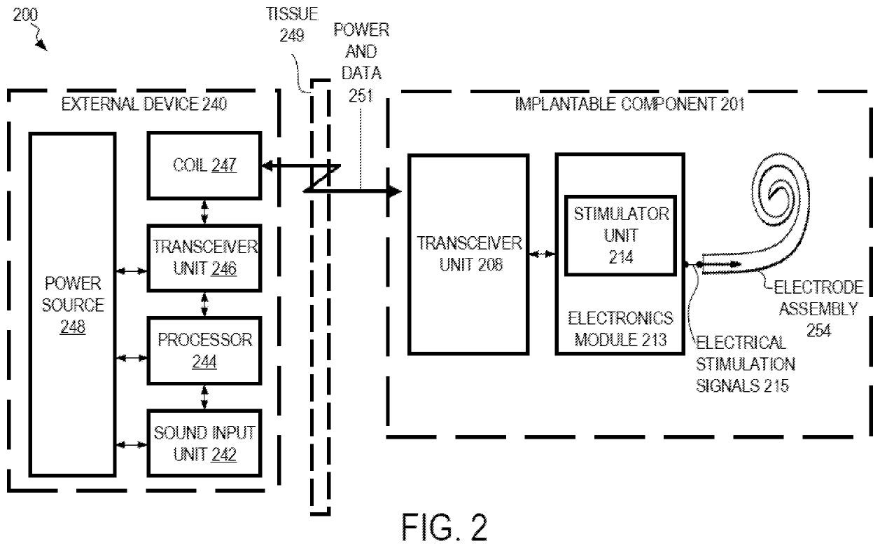

FIG. 2 is a functional block diagram of a cochlear implant system 200 that is usable in an embodiment. The cochlear implant system 200 includes an implantable component 201 (e.g., implantable component 100 of FIG. 1) configured to be implanted beneath a recipient's skin or other tissue 249, and an external device 240 (e.g., the external device 142 of FIG. 1).

The external device 240 can be configured as a wearable external device, such that the external device 240 is worn by a recipient in close proximity to the implantable component, which can enable the implantable component 201 to receive power and stimulation data from the external device 240. As described in FIG. 1, magnets can be used to facilitate an operational alignment of the external device 240 with the implantable component 201. With the external device 240 and implantable component 201 in close proximity, the transfer of power and data can be accomplished through the use of near-field electromagnetic radiation, and the components of the external device 240 can be configured for use with near-field electromagnetic radiation.

Implantable component 201 can include a transceiver unit 208, electronics module 213, which module can be a stimulator assembly of a cochlear implant, and an electrode assembly 254 (which can include an array of electrode contacts disposed on lead 118 of FIG. 1). The transceiver unit 208 is configured to transcutaneously receive power and/or data from external device 240. As used herein, transceiver unit 208 refers to any collection of one or more components which form part of a transcutaneous energy transfer system. Further, transceiver unit 208 can include or be coupled to one or more components that receive and/or transmit data or power. For example, the example includes a coil for a magnetic inductive arrangement coupled to the transceiver unit 208. Other arrangements are also possible, including an antenna for an alternative RF system, capacitive plates, or any other utilitarian arrangement. In an example, the data modulates the RF carrier or signal containing power. The transcutaneous communication link established by the transceiver unit 208 can use time interleaving of power and data on a single RF channel or band to transmit the power and data to the implantable component 201. In some examples, the processor 244 is configured to cause the transceiver unit 246 to interleave power and data signals, such as is described in U.S. Patent Publication Number 2009/0216296 to Meskens. In this manner, the data signal is modulated with the power signal, and a single coil can be used to transmit power and data to the implanted component 201. Various types of energy transfer, such as infrared (IR), electromagnetic, capacitive and inductive transfer, can be used to transfer the power and/or data from the external device 240 to the implantable component 201.

Aspects of the implantable component 201 can require a source of power to provide functionality, such as receive signals, process data, or deliver electrical stimulation. The source of power that directly powers the operation of the aspects of the implantable component 201 can be described as operational power. There are two exemplary ways that the implantable component 201 can receive operational power: a power source internal to the implantable component 201 (e.g., a battery) or a power source external to the implantable component. However, other approaches or combinations of approaches are possible. For example, the implantable component may have a battery but nonetheless receive operational power from the external component (e.g., to preserve internal battery life when the battery is sufficiently charged).

The internal power source can be a power storage element (not pictured). The power storage element can be configured for the long-term storage of power, and can include, for example, one or more rechargeable batteries. Power can be received from an external source, such as the external device 240, and stored in the power storage element for long-term use (e.g., charge a battery of the power storage element). The power storage element can then provide power to the other components of the implantable component 201 over time as needed for operation without needing an external power source. In this manner, the power from the external source may be considered charging power rather than operational power, because the power from the external power source is for charging the battery (which in turn provides operational power) rather than for directly powering aspects of the implantable component 201 that require power to operate. The power storage element can be a long-term power storage element configured to be a primary power source for the implantable component 201.

In some embodiments, the implantable component 201 receives operational power from the external device 240 and the implantable component 201 does not include an internal power source (e.g., a battery)/internal power storage device. In other words, the implantable component 201 is powered solely by the external device 240 or another external device, which provides enough power to the implantable component 201 to allow the implantable component to operate (e.g., receive data signals and take an action in response). The operational power can directly power functionality of the device rather than charging a power storage element of the external device implantable component 201. In these examples, the implantable component 201 can include incidental components that can store a charge (e.g., capacitors) or small amounts of power, such as a small battery for keeping volatile memory powered or powering a clock (e.g., motherboard CMOS batteries). But such incidental components would not have enough power on their own to allow the implantable component to provide primary functionality of the implantable component 201 (e.g., receiving data signals and taking an action in response thereto, such as providing stimulation) and therefore cannot be said to provide operational power even if they are integral to the operation of the implantable component 201.

As shown, electronics module 213 includes a stimulator unit 214 (e.g., which can correspond to the stimulator of FIG. 1). Electronics module 213 can also include one or more other components used to generate or control delivery of electrical stimulation signals 215 to the recipient. As described above with respect to FIG. 1, a lead (e.g., elongate lead 118 of FIG. 1) can be inserted into the recipient's cochlea. The lead can include an electrode assembly 254 configured to deliver electrical stimulation signals 215 generated by the stimulator unit 214 to the cochlea.

In the example system 200 depicted in FIG. 2, the external device 240 includes a sound input unit 242, a sound processor 244, a transceiver unit 246, a coil 247, and a power source 248. The sound input unit 242 is a unit configured to receive sound input. The sound input unit 242 can be configured as a microphone (e.g., arranged to output audio data that is representative of a surrounding sound environment), an electrical input (e.g., a receiver for a frequency modulation (FM) hearing system), and/or another component for receiving sound input. The sound input unit 242 can be or include a mixer for mixing multiple sound inputs together.

The processor 244 is a processor configured to control one or more aspects of the system 200, including converting sound signals received from sound input unit 242 into data signals and causing the transceiver unit 246 to transmit power and/or data signals. The transceiver unit 246 can be configured to send or receive power and/or data 251. For example, the transceiver unit 246 can include circuit components that send power and data (e.g., inductively) via the coil 247. The data signals from the sound processor 244 can be transmitted, using the transceiver unit 246, to the implantable component 201 for use in providing stimulation or other medical functionality.

The transceiver unit 246 can include one or more antennas or coils for transmitting the power or data signal, such as coil 247. The coil 247 can be a wire antenna coil having of multiple turns of electrically insulated single-strand or multi-strand wire. The electrical insulation of the coil 247 can be provided by a flexible silicone molding. Various types of energy transfer, such as infrared (IR), radiofrequency (RF), electromagnetic, capacitive and inductive transfer, can be used to transfer the power and/or data from external device 240 to implantable component 201.

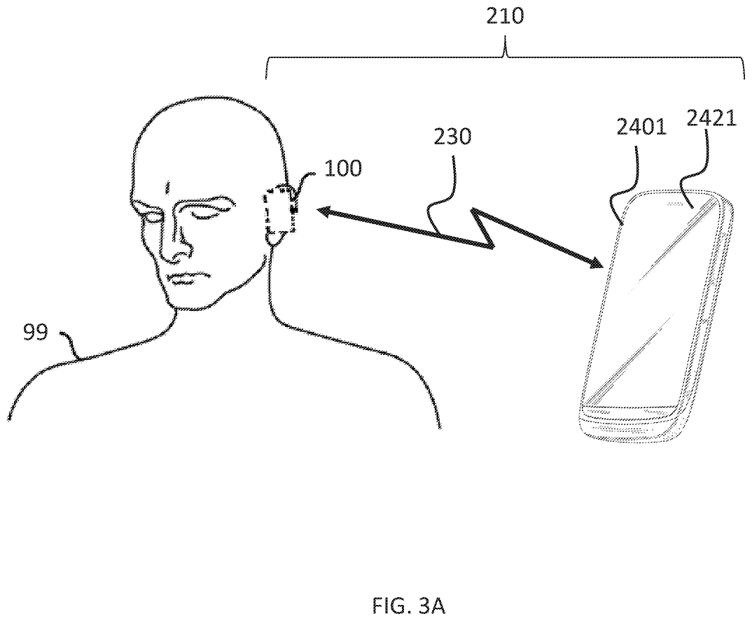

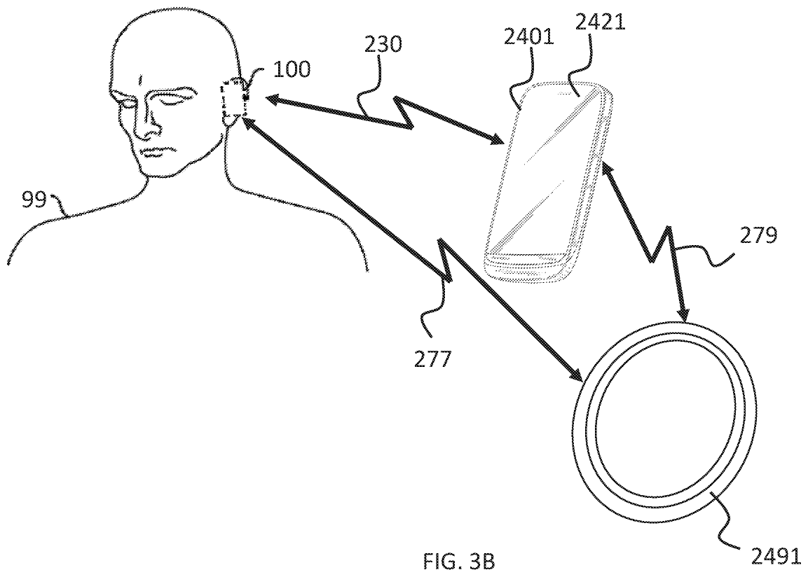

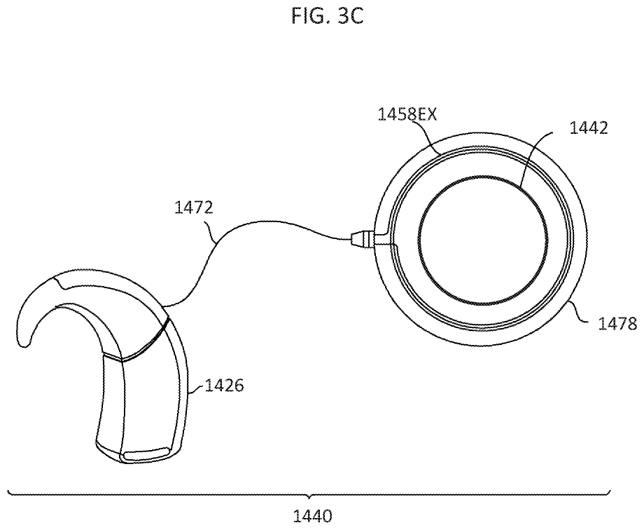

FIG. 3A depicts an exemplary system 210 according to an exemplary embodiment, including hearing prosthesis 100, which, in an exemplary embodiment, corresponds to cochlear implant 100 detailed above, and a portable body carried device (e.g., a portable handheld device as seen in FIG. 2A, a watch, a pocket device, etc.) 2401 in the form of a mobile computer having a display 2421. The system includes a wireless link 230 between the portable handheld device 2401 and the hearing prosthesis 100. In an embodiment, the prosthesis 100 is an implant implanted in recipient 99 (represented functionally by the dashed lines of box 100 in FIG. 3A).

In an exemplary embodiment, the system 210 is configured such that the hearing prosthesis 100 and the portable handheld device 2401 have a symbiotic relationship. In an exemplary embodiment, the symbiotic relationship is the ability to display data relating to, and, in at least some instances, the ability to control, one or more functionalities of the hearing prosthesis 100. In an exemplary embodiment, this can be achieved via the ability of the handheld device 2401 to receive data from the hearing prosthesis 100 via the wireless link 230 (although in other exemplary embodiments, other types of links, such as by way of example, a wired link, can be utilized). As will also be detailed below, this can be achieved via communication with a geographically remote device in communication with the hearing prosthesis 100 and/or the portable handheld device 2401 via link, such as by way of example only and not by way of limitation, an Internet connection or a cell phone connection. In some such exemplary embodiments, the system 210 can further include the geographically remote apparatus as well. Again, additional examples of this will be described in greater detail below.

As noted above, in an exemplary embodiment, the portable handheld device 2401 comprises a mobile computer and a display 2421. In an exemplary embodiment, the display 2421 is a touchscreen display. In an exemplary embodiment, the portable handheld device 2401 also has the functionality of a portable cellular telephone. In this regard, device 2401 can be, by way of example only and not by way of limitation, a smart phone, as that phrase is utilized generically. That is, in an exemplary embodiment, portable handheld device 2401 comprises a smart phone, again as that term is utilized generically.

It is noted that in some other embodiments, the device 2401 need not be a computer device, etc. It can be a lower tech recorder, or any device that can enable the teachings herein.

The phrase “mobile computer” entails a device configured to enable human-computer interaction, where the computer is expected to be transported away from a stationary location during normal use. Again, in an exemplary embodiment, the portable handheld device 2401 is a smart phone as that term is generically utilized. However, in other embodiments, less sophisticated (or more sophisticated) mobile computing devices can be utilized to implement the teachings detailed herein and/or variations thereof. Any device, system, and/or method that can enable the teachings detailed herein and/or variations thereof to be practiced can be utilized in at least some embodiments. (As will be detailed below, in some instances, device 2401 is not a mobile computer, but instead a remote device (remote from the hearing prosthesis 100. Some of these embodiments will be described below).) In an exemplary embodiment, the portable handheld device 2401 is configured to receive data from a hearing prosthesis and present an interface display on the display from among a plurality of different interface displays based on the received data. Exemplary embodiments will sometimes be described in terms of data received from the hearing prosthesis 100. However, it is noted that any disclosure that is also applicable to data sent to the hearing prosthesis from the handheld device 2401 is also encompassed by such disclosure, unless otherwise specified or otherwise incompatible with the pertinent technology (and vice versa).

It is noted that in some embodiments, the system 210 is configured such that cochlear implant 100 and the portable device 2401 have a relationship. By way of example only and not by way of limitation, in an exemplary embodiment, the relationship is the ability of the device 2401 to serve as a remote microphone for the prosthesis 100 via the wireless link 230. Thus, device 2401 can be a remote mic. That said, in an alternate embodiment, the device 2401 is a stand-alone recording/sound capture device.

It is noted that in at least some exemplary embodiments, the device 2401 corresponds to an Apple Watch™ Series 1 or Series 2, as is available in the United States of America for commercial purchase as of Jan. 10, 2021. In an exemplary embodiment, the device 2401 corresponds to a Samsung Galaxy Gear™ Gear 2, as is available in the United States of America for commercial purchase as of Jan. 10, 2021. The device is programmed and configured to communicate with the prosthesis and/or to function to enable the teachings detailed herein.

In an exemplary embodiment, a telecommunication infrastructure can be in communication with the hearing prosthesis 100 and/or the device 2401. By way of example only and not by way of limitation, a telecoil 2491 or some other communication system (Bluetooth, etc.) is used to communicate with the prosthesis and/or the remote device. FIG. 2B depicts an exemplary quasi-functional schematic depicting communication between an external communication system 2491 (e.g., a telecoil), and the hearing prosthesis 100 and/or the handheld device 2401 by way of links 277 and 279, respectively (note that FIG. 3B depicts two-way communication between the hearing prosthesis 100 and the external audio source 2491, and between the handheld device and the external audio source 2491—in alternate embodiments, the communication is only one way (e.g., from the external audio source 2491 to the respective device)). It is noted that unless otherwise noted, the embodiment of FIG. 3B is applicable to any body worn medical device/implanted device disclosed herein in some embodiments.

FIG. 3C depicts an exemplary external component 1440. External component 1440 can correspond to external component 142 of the system 10 (it can also represent other body worn devices herein/devices that are used with implanted portions). As can be seen, external component 1440 includes a behind-the-ear (BTE) device 1426 which is connected via cable 1472 to an exemplary headpiece 1478 including an external inductance coil 1458EX, corresponding to the external coil of FIG. 1. As illustrated, the external component 1440 comprises the headpiece 1478 that includes the coil 1458EX and a magnet 1442. This magnet 1442 interacts with the implanted magnet (or implanted magnetic material) of the implantable component to hold the headpiece 1478 against the skin of the recipient. In an exemplary embodiment, the external component 1440 is configured to transmit and/or receive magnetic data and/or transmit power transcutaneously via coil 1458EX to the implantable component, which includes an inductance coil. The coil 1458X is electrically coupled to BTE device 1426 via cable 1472. BTE device 1426 may include, for example, at least some of the components of the external devices/components described herein.

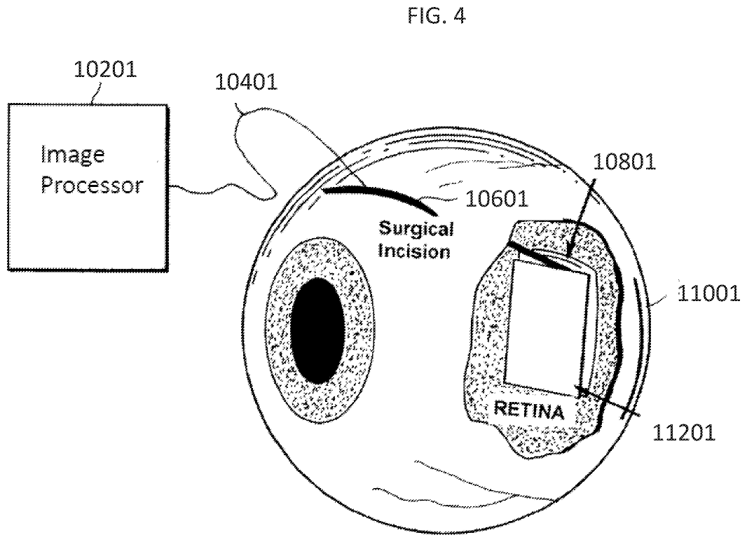

FIG. 4 presents an exemplary embodiment of a neural prosthesis in general, and a retinal prosthesis and an environment of use thereof, in particular, the components of which can be used in whole or in part, in some of the teachings herein. In some embodiments of a retinal prosthesis, a retinal prosthesis sensor-stimulator 10801 is positioned proximate the retina 11001. In an exemplary embodiment, photons entering the eye are absorbed by a microelectronic array of the sensor-stimulator 10801 that is hybridized to a glass piece 11201 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 10201 is in signal communication with the sensor-stimulator 10801 via cable 10401 which extends through surgical incision 00601 through the eye wall (although in other embodiments, the image processor 10201 is in wireless communication with the sensor-stimulator 10801). The image processor 10201 processes the input into the sensor-stimulator 10801 and provides control signals back to the sensor-stimulator 10801 so the device can provide processed output to the optic nerve. That said, in an alternate embodiment, the processing is executed by a component proximate with or integrated with the sensor-stimulator 10801. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 10801 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

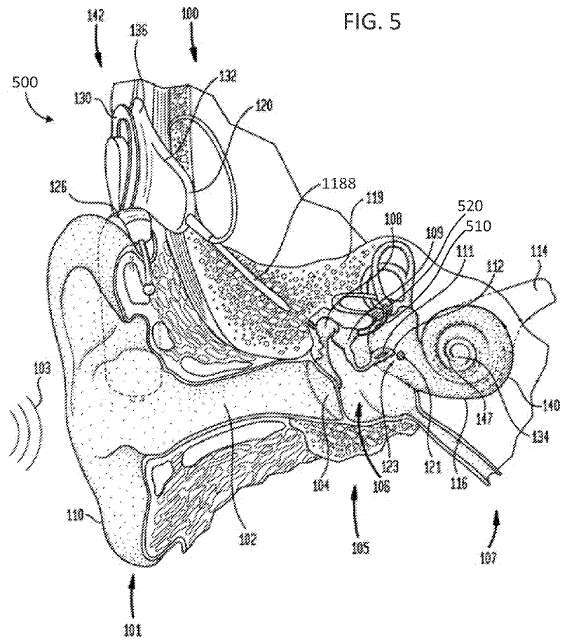

FIG. 5 depicts an exemplary vestibular implant 500 according to one example. Some specific features are described utilizing the above noted cochlear implant of FIG. 1 in contacts for the various elements. In this regard, as noted above, some features of a cochlear implant are utilized with vestibular implants. That is, embodiments herein can use cross-over technologies. In the interest of textual and pictorial economy, various elements of the vestibular implant that generally correspond to the elements of the cochlear implant above are referenced utilizing the same numerals. Still, it is noted that some features of the vestibular implant 500 will be different from that of the cochlear implant above. By way of example only and not by way of limitation, there may not be a microphone on the behind-the-ear device 126. Alternatively, sensors that have utilitarian value in the vestibular implant can be contained in the BTE device 126. By way of example only and not by way of limitation, motion sensors can be located in BTE device 126. There also may not be a sound processor in the BTE device. Conversely, other types of processors, such as those that process data obtained from the sensors, will be present in the BTE device 126. Power sources, such as a battery, will also be included in the BTE device 126. Consistent with the BTE device of the cochlear implant of FIG. 1, a transmitter/transceiver will be located in the BTE device or otherwise in signal communication therewith.

The implantable component includes a receiver stimulator in a manner concomitant with the above cochlear implant. Here, vestibular stimulator comprises a main implantable component 120 and an elongate electrode assembly 1188 (where the elongate electrode assembly 1188 has some different features from the elongate electrode assembly 118 of the cochlear implant, some of which will be described shortly). In some embodiments, internal energy transfer assembly 132 and main implantable component 120 are hermetically sealed within a biocompatible housing. In some embodiments, main implantable component 120 includes a processing unit (not shown) to convert data obtained by sensors, which could be on board sensors implanted in the recipient, into data signals.

Main implantable component 120 further includes a stimulator unit (also not shown) which generates electrical stimulation signals based on the data signals. The electrical stimulation signals are delivered to the recipient via elongate electrode assembly 1188.

It is briefly noted that while the embodiment shown in FIG. 5 represents a partially implantable vestibular implant, embodiments can include a totally implantable vestibular implant, such as, where, for example, the motion sensors are located in the implantable portion, in a manner analogous to a cochlear implant.

Elongate electrode assembly 1188 has a proximal end connected to main implantable component 120, and extends through a hole in the mastoid 119, in a manner analogous to the elongate electrode assembly 118 of the cochlear implant, and includes a distal end that extends to the inner ear. In some embodiments, the distal portion of the electrode assembly 1188 includes a plurality of leads 510 that branch out away from the main body of the electrode assembly 118 to electrodes 520. Electrodes 520 can be placed at the base of the semicircular ducts as shown in FIG. 5. In an exemplary embodiment, one or more of these electrodes are placed in the vicinity of the vestibular nerve branches innervating the semicircular canals. In some embodiments, the electrodes are located external to the inner ear, while in other embodiments, the electrodes are inserted into the inner ear. Note also while this embodiment does not include an electrode array located in the cochlea, in other embodiments, one or more electrodes are located in the cochlea in a manner analogous to that of a cochlear implant.

Embodiments can be directed to, but are not limited to, humans and mammals who experience/are afflicted with, vestibular dysfunction, including for example, bilateral vestibular dysfunction. Embodiments can include using the device of FIG. 5 or a variation thereof, or a related device, to treat BVD (any disclosure herein of BVD corresponds to an alternat disclosure of unilateral vestibular dysfunction, and vice versa, unless otherwise noted, or in fact, any vestibular dysfunction that can cause balance difficulties in a human, or in some embodiments, general balance difficulties). In some scenarios, such people can show postural instability and gait difficulties. Some embodiments include an implant that provides one or more stimulating electrodes for medium and/or long-term stimulation on one or more inferior vestibular nerve and, in some embodiments, by way of example, deliver a constant train of electrical pulses, such as high-frequency electrical pulses, by one or more of those electrodes to one or more of those nerves. In an embodiment, the vestibular stimulator, such as the device of FIG. 5, can deliver 500, 600, 700, 800, 900, 1000, 1100, 1200, 1300, 1400, 1500, 1600, 1700, 1800, 1900, 2000 or more, or less, or any value or range of values therebetween in 1 increment (e.g., 1111, 678, 1776, 1234 to 1818, etc.) mono or biphasic pulses per second. The electrical stimulation can be provided by an electrode at a location where, for example, the electrical stimulation is stimulating saccular afferents, including mainly stimulating saccular afferents.

Embodiments include providing the electrical stimulation in a manner that can improve the human's postural stability and/or gait performance, which can be measured by Computerized Dynamic Posturography (CDP) and the Dynamic Gait Index (DGI) to evaluate the efficacy of such (which is not needed to implement embodiments-this is a control that can be used by one of skill in the art to evaluate the definitiveness of performance. Embodiments can “improve” peripheral vestibular function by the stimulation. Efficacy can be evaluated, in some embodiments, the extent to which the human has “need” or even desires assistance in walking and stabilizing themselves. To be clear, embodiments can, but do not need to, be linked to the “saccular substitution” concept, and not all embodiments relate/cause neural activity generated by the electrical stimulation to substitute for the reduced or absent constant saccular afferent activity from the saccular macula. Any device, system, and/or method that enhances balance, gate, or otherwise modifies balance and/or gate, by way of electrical stimulation, or by way of chemical application for that matter, can be used in some embodiments, providing that the art enables such, unless otherwise noted.

Indeed, in view of the ability of at least some of the teachings detailed herein to improve postural stability and/or gait performance, embodiments can also be directed towards utilizing one or more or all of the teachings detailed herein with respect to treatment of movement disorders and/or motor disorders in addition to balance disorders, at least where the two are mutually exclusive or otherwise sufficiently exclusive enough to be considered separate disorders. Indeed, embodiments include devices, systems and/or methods for treating/of treating vestibular function impairments/disorders, regardless of the ultimate effects of that disorder (balance or motor function impairment, etc.) Embodiments can include applying the electrical stimulation techniques detailed herein to reduce the magnitude of or otherwise control tremors (whole body or localized, such as in the arms or hands or legs or feet, etc.) that are for example involuntary. These embodiments can leverage the nexus that exists in some medical scenarios where vestibular function and motor functions are connected or otherwise correlated to one another.

In view of the utilitarian value of applying electrical stimulation (or other type of stimulation for that matter as complemented herein) to tissue to treat movement disorders, in the interest of textual economy, any disclosure herein of implementing the teachings herein to treat balance disorders corresponds to an alternate disclosure of treating movement disorders/motor disorders unless otherwise noted providing that the art enables such. Moreover, in the interest of textual economy, any disclosure herein of implementing the teachings herein to treat balance disorders corresponds to an alternate disclosure of treating vestibular function-impairment/vestibular function disorders. To be clear, any device, system and/or method action disclosed herein can be applicable to a device, system and/or method, respectively, of treating a movement disorder/motor disorder/vestibular function disorder in a human providing that the art enables such. By way of example only and not by way of limitation, any disclosure of improving balance corresponds to an alternate disclosure of improving motor function and/or movement function. Further, any disclosure of improving balance corresponds to an alternate disclosure of improving posture and/or gait (which is movement, albeit a more specific type thereof). And any disclosure of improving balance corresponds to a disclosure of improving vestibular function. In this regard, any disclosure of a balance sensory medical device herein corresponds to an alternate disclosure of a vestibular function medical device, etc., and any method of treating balance corresponds to an alternate disclosure of a method of treating a vestibular function.

It is noted that embodiments can include devices, systems and/or methods for and of controlling motor function and movements, and otherwise providing therapy to a human as detailed herein variously and in other manners, by stimulating other portions of the body other than the vestibula. Accordingly, the embodiments disclosed herein relating to the devices, systems and/or methods of evaluating the efficacy of the medical device and/or providing an indication that the system/device is activated and/or not activated and/or determining whether the system/device is functioning and/or not functioning, etc., and the associated teachings thereabout, correspond to an alternate disclosure of utilizing those teachings with medical devices that stimulate parts of the body other than the vestibular system, such as medical devices disclosed in the preceding paragraph, and otherwise medical devices that address motor function impairment/movement disorders, etc., provided that the art enables such, unless otherwise noted. In this regard, the aforementioned utilitarian value associated with providing an indication to the recipient as to whether or not a stimulation device is functioning, etc., can have utilitarian value with respect to these other medical devices. For example, a human that relies on a medical device that improves motor control could injure himself or herself or others if the device is not functioning in a scenario where the recipient of the medical device does not know that the system is not functioning. By more specific example, a human that is utilizing a sharp knife to cut food who believes that a medical device that remedies at least partially a disorder associated with motor control is functioning, but where, in reality, the device is not functioning or otherwise functioning at an efficacy level below that which is expected, could cut himself or herself or stab himself or herself by accident because of a degraded motor function relative to that which would otherwise be the case if the device was functioning in an efficacious manner. This could also be the case with respect to driving, walking, and other activities detailed herein in other activities.

Some embodiments use electrical stimulation to activate or otherwise stimulate the descending spinal pathways as well. Embodiments can include saccular stimulation as noted above. Embodiments can include deep-brain stimulation, providing that such can affect balance, such as simulation by electrodes in/at the basal ganglia acting to help alleviate the symptoms of the human vis-à-vis balance and/or gait. Embodiments can include stimulating the spine as well, providing such has an efficacious effect on balance and/or gait. The devices above and/or modified devices or related devices can be used to provide such electrical stimulation.

Embodiments include the application of electrical stimulation, including constant electrical stimulation, of the vestibular nerves, such as the inferior vestibular never, in humans, to improve or at least attempt to improve balance and/or gait of the human who suffers from balance and/or gait problems, including such problems that are a result of vestibular dysfunction. While the embodiments above have been directed to an implanted device, embodiments can include external devices that apply electrical stimulation to the surface of the skin from external electrodes (and thus can be a non-implanted/implantable device).

Embodiments include devices, systems, and methods that utilize stimulation (electrical and/or chemical) to humans that can have, at least in some embodiments, vestibular dysfunction (bilateral or unilateral). In some of these humans, there is at least some residual peripheral vestibular function, but the level of such falls below the international accepted standard for vestibular dysfunction, such as the standard for bilateral vestibular dysfunction. In an embodiment, the level is at least 10, 15, 20, 25, 30, 35, 40, 45, 50, 55, 60, 65, 70, 75, or 80% or more, or any value or range of values therebetween in 1% increments reduction in the accepted standard and/or the normal level of function, and/or normal results with respect to postural stability and/or gait using the DGI and/or CDP, etc. (providing that there is some neurological issue that creates the results).

Embodiments can be implemented in a dedicated vestibular stimulation implant, or a modified cochlear implant, or an implant that uses a modified cochlear implant electrode and/or a plurality of electrodes or a modified cochlear implant array. In an embodiment, the electrode(s) are implanted on, at or proximate a branch of the vestibular nerve in one ear or both ears. The exact location of the electrode(s) can depend on particular anatomical considerations at surgery. Embodiments can include implanting the electrode(s)/having the electrode(s) being implanted, very close to the inferior vestibular nerve containing afferents from the saccular macula and posterior semicircular canal. This can be verified from a CT scan or some other non-invasive inspection regime. Embodiments can have the electrode(s) within 10, 9, 8, 7, 6, 5, 4, 3, 2.5, 2, 1.5, 1, 0.75, 0.5, 0.25, 0.2, 0.15, or 0.1 mm, or less, or any value or range of values therebetween in 0.01 mm increments, from the inferior vestibular nerve, such as the nerve containing afferents from the saccular macula and posterior semicircular canal. The electrode(s) locations can be otolithic and/or saccular afferents. Embodiments can utilize vestibular stimulation and/or saccular stimulation. But any stimulation that can treat gait and/or posture and/or balance problems can be used in some embodiments.

Embodiments include devices, systems, and/or methods of applying a train of pulses, such as a constant train of pulses, such as at high-frequency or medium frequency, and maintained continuously and/or semi continuously while the recipient is involved in walking activities and/or activities that involve balance and/or coordination, etc. That said, in some embodiments, the application of electrical stimulation can be applied only when needed/utilitarian, and thus need not be continuous.

Embodiments can include a stimulation device that includes 1, 2, 3, 4, 5, 6, 7, 8, 9 or 10 or more electrodes or any values or range of values therebetween in 1 increment (4-8, 2-8, etc.). The stimulation device, which again can be based on a cochlear implant/a modified cochlear implant, or a vestibular device, or any device that can provide electrical stimulation to enable the teachings herein, can provide a mono phase and/or a biphasic series of pulses, such as pulses having 5, 10, 15, 20, 25, 30, 35, 40, 45, 50, 55 or 60 or any value or range arise therebetween in one increment microseconds per phase, and can be delivered at any of the frequency's detailed above or any other frequencies for that matter that can enable the teachings detailed herein. In an embodiment, one, two, three, four, five, or more electrodes are activated so that the stimulus is essentially a constant train of pulses. In an embodiment, the electrical stimulation activates the vestibular afferents at a relatively high rate, thus duplicating the constant barrage of action potentials that a normal human would receive.

Embodiments include providing stimulus to the human in a manner that influences the cerebellum in the control of balance and/or locomotion and/or coordination. Embodiments can be applied where there is cerebellar damage and/or cerebellar loss in the human. Embodiments can be applied to humans that show for example excessive or diminished responses to perturbations, with inadequate (e.g., poor) control of equilibrium during motion and abnormal (statistically significant) oscillation of the trunk. The patient can experience gait ataxia with distinctive features, including variable foot placement, irregular foot trajectories, wide base of support, a veering path of movement, and abnormal inter-joint coordination. Embodiments include stimulating the human so that the medial cerebellar zone can integrate spinal and vestibular inputs and influence motor pathways for walking.

It is noted that the various medical devices herein can be of the type that recreate a feeling of balance/coordination, etc., by stimulating with an electrical signal and/or chemical that is related to information gathered with accelerometer and/or gyroscope or other device that can enable an orientation of the recipient/human to be evaluated/determined. It is noted that the various medical devices herein can be of the type that inhibits or suppresses the balance function/coordination function by stimulation with an electrical signal, such as an unmodulated electrical signal and/or chemical. Embodiments can include a medical device, implant or otherwise, that is configured to do one or both (the medical device can be a system that is integrated, for example, and that can determine whether or not one type of treatment is more utilitarian than the other, or otherwise more efficacious than the other, which could be determined automatically by the system or by a remote healthcare professional and/or by the human himself or herself; the human can have a “feel” for which type of treatment has the desired results for a given scenario, and this can be based on empirical and/or statistical and/or analytical data). In an embodiment, the apparatuses detailed herein are configured recreate a feeling of balance in the recipient by stimulation of tissue of the recipient using the electrical signal(s) and configured to enhance and/or provide signals from the vestibula (or elsewhere for that matter, as noted above) that travel to the brain by stimulation of tissue of the recipient using the electrical signal(s).

It is further noted that the various medical devices detailed herein can be of the sensory substitution variety. In an embodiment, there can be a device that produces an acoustic stimulus or some other stimulus that is related to information gathered with a body worn/carried device (e.g., a device that includes an accelerometer, gyroscope, or some other device that can sense orientation of a human etc.).

Embodiments can include systems and methods for sensory substitution by suppressing one sensory channel and providing signals via another. In one example, a system suppresses a dysfunctional vestibular system and provides substitute vestibular information via another sensory channel. Dysfunctional balance information from a recipient's vestibular system can be suppressed using electrical stimulation. The electrical stimulation can be provided to the otolith region, semicircular canals, vestibular nerve or another portion of the recipient's vestibular system. Balance information that would normally be provided by a healthy vestibular system (e.g., how the recipient is positioned with respect to gravity, such as rotation along pitch and roll axes) is provided via stimulation of another sensory channel. Such sensor channels can include visual, audible, or tactile sensory channels. For example, audible percepts can be generated via an auditory prosthesis (e.g., a cochlear implant providing electrical stimulation in the recipient's cochlea). By suppressing one sensory channel and providing stimulation via another, one sense can be substituted for another. While described herein primarily in the context of vestibular sensory substitution, sensory substitution can be extended to other sensory systems.

Indeed, embodiment can include a medical device where there is an architecture for a combined auditory system and sensory substitution system. In an example, sensory substitution is delivered as perceptible auditory cues that are provided via intracochlear electrodes. The sensory substitution cues can be provided via one or more dedicated intracochlear electrode channels, rather than being superimposed on all hearing channels. In such examples, the remaining electrodes can deliver standard cochlear implant stimulation (e.g., to cause auditory precepts to make up for a dysfunctional auditory system). Balance signals that can substitute for the dysfunctional vestibular system can originate from one or more accelerometers, magnetometers other sensors, or combinations thereof that pass pitch, roll and yaw information to a balance signal generator. The balance signals that substitute for the dysfunctional vestibular system can then be injected into the cochlear stimulation signal processing path in such a manner as to not interfere with (or be interfered by) other signal channels. Thus, while some of the sound processing path can be shared between balance signals and sound input signals (e.g., from a microphone or other sound source), some of the processing path can be exclusive to the balance input signals. In at least some examples, some of the processing path can be exclusive to the sound input signals and some of the processing path can be shared by both the sound input signals and the balance input signals.

The various balance signals that can be used to substitute for a dysfunctional vestibular system include movement or position compared to gravity that is used as an indicator of stability of the recipient. Such a signal can be used to provide information allowing the recipient to quickly recover from stumble or incident of balance failure, which can aid in fall prevention. In another example, gait information is extracted from one or more sensors placed at different locations on the recipient's body (e.g., in a smart watch, phone, gait monitor, step counter, or another device having one or more sensors). Extraction of gait information can be used to predict falls. Fall prediction can be used in combination with fall prevention techniques by, for example, providing balance substitution when gait analysis indicates that there is a risk of falling.

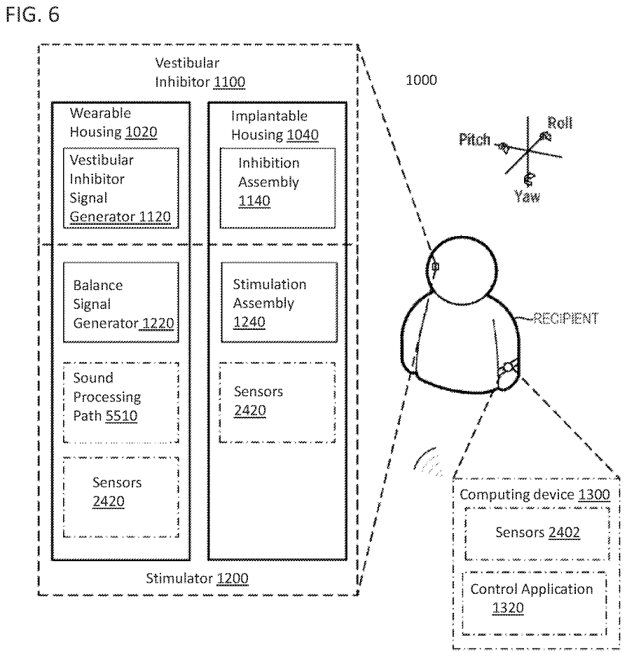

FIG. 6 illustrates an example system 1000 for treating a balance dysfunction of a recipient. The illustrated system 1000 includes a vestibular inhibitor 1100 and a stimulator 1200.

The vestibular inhibitor 1100 is a portion of the system 1000 configured to inhibit the recipient's vestibular system. The vestibular inhibitor 1100 can include a vestibular inhibitor signal generator 1120 and an inhibition assembly 1140, which can be disposed in the same or separate housings.

The vestibular inhibitor signal generator 1120 can be a component that controls the stimulation provided by the inhibition assembly 1140, such as by being or including one or more processors that provide signals. For example, the vestibular inhibitor signal generator 1120 can be configured to provide stimulation signals to the inhibition assembly 1140.

The inhibition assembly 1140 can take any of a variety of forms. The inhibition assembly 1140 can include one or more stimulation electrodes. The inhibition assembly 1140 can be or include an implantable assembly configured to apply electrical stimulation to an otolith region, semicircular canals, other vestibular tissue of the recipient, or combinations thereof using the one or more electrodes. The electrical stimulation can inhibit the signals provided by the vestibular system to reduce the perception of signals produced by a portion of the vestibular system. For example, where the vestibular system of the recipient is dysfunctional, the stimulation provided by the vestibular inhibitor 1100 can be sufficient to reduce or eliminate the perception of dysfunctional signals by the recipient. In some examples, this is achieved by preventing the vestibular system from producing signals or by causing the signals that are produced by the vestibular system to be noisy or otherwise have properties that cause the signals to be disregarded by the recipient.

Additional example implementations of a vestibular stimulator that can act as one or both of the vestibular inhibitor signal generator 1120 and the inhibition assembly 1140 are described in relation to European Patent Application No. 19382629.4 and European Patent Application No. 19382632.8, both of which were filed on Jul. 24, 2019. Embodiments can include one or more or all of the features therein.

The stimulator 1200 is a portion of the system 1000 configured to cause a sensory percept (e.g., audio, visual, or tactile precepts) for the recipient. Such a sensory percept can be used to, for example, provide balance compensation signals to the recipient via one or more non-vestibular sensory channels of the recipient. Balance compensation signals can be signals that cause sensory percepts configured to compensate for a dysfunctional vestibular system. For instance, the balance compensation signals can provide balance information relating to the percepts that would be provided by a normally functioning vestibular system, such as information regarding balance, equilibrium, and orientation in space, among others.

The stimulator 1200 can be configured to target one or more non-vestibular sensor channels of the recipient with stimulation to convey balance information. The stimulator 1200 can include a balance signal generator 1220 and a stimulation assembly 1240, disposed in a same or separate housings.

The balance signal generator 1220 can be a component configured to generate one or more balance compensation output signals to cause stimulation via the stimulation assembly 1240. The balance compensation output signals can be configured to compensate a vestibular deficiency, such as by providing precepts indicative of balance information in a manner that bypasses a defective vestibular system of a recipient.

The stimulation assembly 1240 can be a component configured to cause one or more sensory percepts in the recipient to provide the balance information based on the balance compensation output signals. For example, the sensory percepts can provide the balance information to the recipient via one or more non-vestibular sensory channels of the recipient. The one or more sensory channels can include, for example, a visual sensory channel, an auditory sensory channel, a tactile sensory channel, other sensor channels, or combinations thereof. Various characteristics of these sensory channels can be modified to convey different components of balance information. For example, providing balance information regarding rotation about a first axis (e.g., a roll axis) can be performed using a first characteristic, and providing balance information regarding rotation about a second axis (e.g., a pitch axis) can be performed using a second characteristic. In another example, providing balance information regarding rotation about a first axis (e.g., a roll axis) can be performed using a first sensory channel, and providing balance information regarding rotation about a second axis (e.g., a pitch axis) can be performed using a second sensory channel.

Where the sensory channel is a visual sensory channel, the stimulation assembly 1240 can be configured to cause the recipient to experience visual percepts that convey the balance information. The balance signal generator 1220 can provide signals to the stimulation assembly 1240 to vary characteristics of the visual percept to convey the balance information. The visual characteristics can include, for example, characteristics of light provided by a set of one or more lights that make up the stimulation assembly 1240 (e.g., LED lights), such as color, brightness, blinking frequency, location, pattern, other characteristics, or combinations thereof. In an example, the stimulation assembly 1240 includes a display (e.g., an LCD display) that can show balance information in any of a variety of forms (e.g., a visual diagram or textual description). The stimulator 1200 can be configured to provide such information visually by, for example, disposing one or more light emitting elements of the stimulation assembly 1240 proximate the recipient's eyes such that the light emitting elements are disposed in the recipient's field of view. The stimulator 1200 can be configured as a wearable headset (e.g., shaped like a pair of eyeglasses). In examples, the stimulator 1200 can directly stimulate portions of the recipient's visual system, such as with a visual prosthesis. In such an example, the stimulation assembly 1240 can be an implantable component configured to provide electrical stimulation to the recipient to cause visual percepts.

Where the sensory channel is a tactile sensory channel, the stimulation assembly 1240 can be configured to cause tactile percepts that are indicative of the balance information. In an example, the stimulation assembly 1240 can include one or more vibratory actuators that vibrate the recipient's skin to convey the balance information tactilely. The balance signal generator 122 can provide signals to the stimulation assembly 1240 to vary characteristics of the tactile percept to convey the balance information. The characteristics modifiable to indicate balance information can include, for example, vibration strength, vibration frequency, and vibration location, among others.

Where the sensory channel is an audio sensory channel, the stimulation assembly 1240 can be configured to cause audio percepts in the recipient that are indicative of the balance information. In an example, the stimulation assembly 1240 can be a headset with speakers. The stimulator 1200 can be a wearable or implantable auditory prosthesis medical device, such as a bone conduction device or a cochlear implant. In such examples, the stimulation assembly 1240 can be or include a vibratory bone conduction actuator or an electrode assembly of a cochlear implant. The balance signal generator 1220 can provide signals to the stimulation assembly 1240 to vary characteristics of the audio percept to convey the balance information. The characteristics modifiable to indicate balance information can include, for example, loudness, pitch, stimulation frequency, location (e.g., left or right side), other characteristics, or combinations thereof. In addition to or instead of tones, the audio percepts can be audio descriptions, such as can be provided by a text-to-speech system describing the balance information.

The balance compensation signals can be generated to cause precepts that convey balance information relating to movement about one or more of pitch, roll, or yaw axes. Rotation about the pitch axis can relate to the recipient's head tilting up and down (e.g., in a nodding motion). Rotation about the roll axis can relate to the recipient's head tilting left or right. Rotation about the yaw axis can relate to the recipient's head rotating left or right. As an example, implementation of the stimulator 1200 can provide audio signals at a first frequency (e.g., corresponding to the pitch Di) to represent a positive rotation about the roll axis and at a second frequency (e.g., corresponding to the pitch CO to represent a negative rotation about the roll axis. A degree of rotation can be represented by changing a volume of the audio signal provided. For instance, a volume can be approximately 0 dB when the rotation is approximately 0 degrees and can increase to approximately 60 dB as the rotation approaches 90 degrees. As the recipient becomes accustomed to such signals indication rotation, the signals can substitute for a dysfunctional vestibular system of the recipient. In some examples, the stimulator 1200 can further include a sound processing path 5510. The balance signal generator 122 can be configured to inject balance compensation output signals into the sound processing path 5510, such as is described in more detail in relation to FIG. 5 herein. Audible percepts are one of a variety of kinds of ways such information can be provided. The stimulator 1200 can take any of a variety of forms.

While the system 1000 can be a single-purpose system (e.g., to solely treat balance dysfunctions by inhibiting vestibular organs and providing balance signals). The system can be a multi-purpose system, such as by the stimulator 1200 providing sensory compensation for multiple sensory systems of the recipient. For example, in addition to providing compensation for a dysfunctional vestibular system, the stimulator 1200 can cause stimulation to compensate for a dysfunctional visual or auditory system of the recipient. In such an example, the balance signal generator 1220 can be in addition to a signal generator to treat the sensory defect. For instance, the stimulator 1200 can be an auditory prosthesis configured to cause hearing percepts in the recipient that are indicative of the auditory environment around the recipient. Such a stimulator 1200 can further include a sound processing path configured to convert an environmental sound input signal into an auditory stimulation signal to cause stimulation via the stimulation assembly 1240. The balance signal generator 1220 can inject a balance information output signal into the sound processing path to cause a hearing percept in the recipient that is indicative of the balance information.

As described above, the various components of the system 1000 can be disposed in same or separate housings. As illustrated, the system 1000 can include a wearable housing 1020 in which the vestibular inhibitor signal generator 1120, balance signal generator 1220, and the sound processing path 5510 are disposed. The wearable housing 1020 can be configured to be worn by the recipient, such as via a headband, magnetic connection, hair clip, or via another technique. As further illustrated, the system 1000 can include an implantable housing 1040. The implantable housing 1040 can at least partially include the inhibition assembly 1140 and the stimulation assembly 1240. For example, the assemblies 1140, 1240 can extend from the implantable housing 1040. The implantable housing 1040 can be constructed from or coated with a biocompatible material. In some examples, the implantable housing 1040 further includes one or more of the vestibular inhibitor signal generator 1120, the balance signal generator 1220, and the sound processing path 5510. While the various components can be separated into a wearable housing 1020 and an implantable housing 1040, in some examples, the components can be disposed entirely in the wearable housing 1020 or the implantable housing 1040. For example, some implementations can implement the vestibular inhibitor 1100 and the stimulator 1200 as a totally-implantable device.

As illustrated, there is one stimulator 1200 and one vestibular inhibitor 1100 disposed on one side of the recipient's head. In other examples, the recipient can have multiple different stimulators 12000 and vestibular inhibitors 1100. In an example, there is a bi-lateral configuration where there are both left-and right-side vestibular inhibitors 1100 and left-and right-side stimulators 1200. Such components can be configured to stimulate respective left and right vestibular or other tissue of the recipient. In some examples, the multiple components can cooperate with each other to provide substantially the same or different stimulation. In some examples, the sidedness of the stimulation (e.g., more intense signals on one side rather than the other) can indicate a particular balance state.

As illustrated, some examples of the system 1000 can further include one or more sensors 2420 disposed in various locations throughout the system 1000. The sensors 2420 can be, for example, one or more sensors for detecting data used for the balance or gait information, such as accelerometers, gyroscopes, piezoelectric sensors, other sensors, or combinations thereof. Additional example sensors 2420 include physiological sensors, such as heartbeat, galvanic skin response sensors, blood pressure sensors, electromyography sensors, other sensors, or combinations thereof. Still further examples of the sensors 2420 include microphones and light sensors, among others. The sensors 2420 can include components disposed within or connected to (e.g., via wired or wireless connections) the components of the system 1000. In some examples, the sensors 2420 include software sensors, such as software that obtains data from one or more of the sensors 2420 and produces additional data based thereon. For example, a software sensor can be configured to obtain data from one or more gyroscopes and accelerometers to produce gait data regarding the recipient. The gait data can relate to how the recipient is walking, running, or otherwise moving. Such data can describe whether the recipient is limping, lurching, or otherwise has an abnormal gate that can be indicative of a balance issue.

As further illustrated, some examples of the system 1000 can further include a computing device 1300. The computing device 1300 can be a computing device associated with the recipient of the stimulator 1200. In many examples, the computing device 1300 is a cell phone, tablet, smart watch, step counter, or heart rate monitor, but the computing device 1300 can take other forms. Although described primarily in the context of the recipient, the computing device 1300 can be a computing device owned or primarily used by a parent or caregiver for the recipient. The computing device 1300 can have one or more processors configured to perform operations based on instructions stored in memory of the computing device 1300. The computing device can further include one or more interfaces for interacting with a user (e.g., via a touchscreen) or other devices (e.g., a wireless transceiver). In the illustrated example, the computing device 1300 includes one or more sensors 2420 and a control application 1320.

The control application 1320 can be a computer program stored as computer-executable instructions in memory of the computing device 1300 that, when executed, performs one or more tasks relating to the system 1000. The control application 1320 can cooperate with one or both of the vestibular inhibitor 1100 and the stimulator 1200. For instance, the control application 1320 can control when and how inhibition is provided by the vestibular inhibitor 1100 and when and how signals are provided by the stimulator 1200. In some examples, such control of the functioning of components of the system 1000 can be performed automatically by the control application 1320 or based on input received from a user of the computing device 1300. The control application 1320 can further provide data from one or more signals from sensors 242 of the computing device 1300 to the stimulator 1200 for use by the balance signal generator 1220. The computing device 1300 can connect to one or both of the vestibular inhibitor 110 and the stimulator 1200 using, for example, a wireless radiofrequency communication protocol (e.g., BLUETOOTH). The control application 1320 can transmit or receive data from one or both of the vestibular inhibitor 1100 and the stimulator 1200 over such a connection. Where the stimulator 1200 includes the sound processing path 5510, the control application 1320 can be configured to stream audio as input into the sound processing path 5510, such as from a microphone of the sensors 2420 or an application running on the computing device 1300 (e.g., a video or audio application). In other examples, another application running on the computing device 1300 can stream audio to the sound processing path 5510.

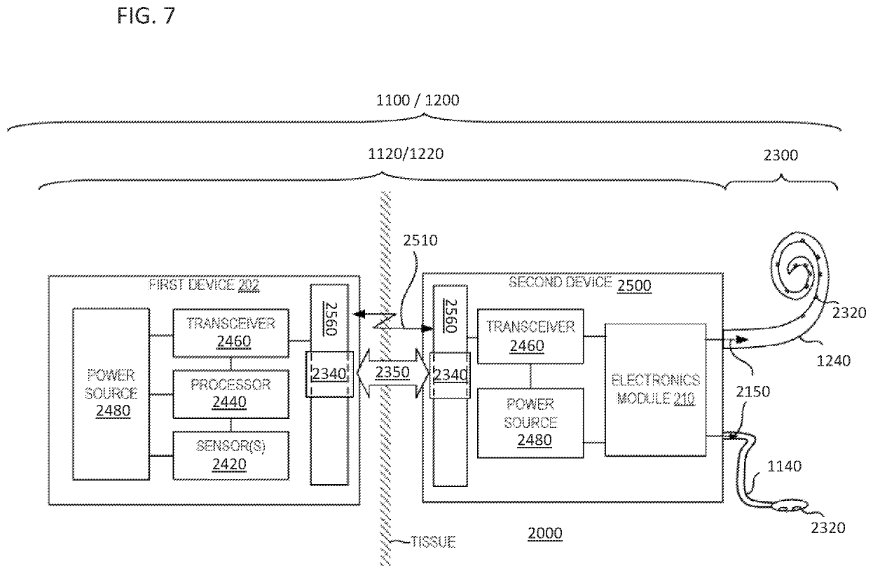

As described above, the components of the system 1000 can take any of a variety of forms. An example apparatus that can be used to implement one or both of the vestibular inhibitor 1100 and the stimulator 1200 is described in FIG. 7. And in this regard, FIG. 7 is a functional block diagram of an example apparatus 2000 that be used to implement one or both of the vestibular inhibitor 1100 and the stimulator 1200. In the illustrated example, the apparatus 2000 includes a first device 2020 acting as an external processor device and a second device 2500 acting as an implanted stimulator device. In examples, the second device 2500 is an implantable stimulator device configured to be implanted beneath a recipient's tissue (e.g., skin). In examples, the second device 2500 includes a biocompatible housing. The first device 2020 can be a device configured to couple with (e.g., wirelessly) the second device 2500 to provide additional functionality, such as stimulation control signals or charging. While the apparatus 2000 is shown as having both implantable and external components, implementations of the apparatus 2000 can be entirely external or entirely implantable.

In the illustrated example, the first device 2020 includes one or more sensors 2420, a processor 2440, a transceiver 2460, and a power source 2480. The one or more sensors 2420 can be units configured to produce data based on sensed activities. In an example where the stimulation system 200 is an auditory prosthesis system, the one or more sensors 2420 can include sound input sensors, such as a microphone, a telecoil, wireless audio sources (e.g., a BLUETOOTH transceiver), an electrical input for an FM hearing system, and/or another component for receiving sound input. Where the stimulation system 0200 is a visual prosthesis system, the one or more sensors 2420 can include one or more cameras or other visual sensors. The processor 2440 can be a component (e.g., a central processing unit) configured to control stimulation provided by the second device 2500. The stimulation can be controlled based on data from the sensor 2420, a stimulation schedule, or other data. Where the stimulation system 2000 implements an auditory prosthesis, the processor 2440 can be configured to convert sound signals received from the sensor(s) 2420 (e.g., acting as a sound input unit) into external device signals 2510, using, for example, a sound processing path as is described elsewhere herein. The transceiver 2460 is a component configured to send signals 2510, such as power signals, data signals, other signals, or combinations thereof (e.g., by interleaving the signals). The transceiver 2460 can be configured to receive power or data. Stimulation signals can be generated by the processor 2440 and transmitted, using the transceiver 2460, to the second device 2500 for use in providing stimulation.

In the illustrated example, the second device 2500 includes an electronics module 2100, a stimulator assembly 2300, a transceiver 2460, a power source 2480, and a coil 2560. The second device 2500 further includes a hermetically sealed, biocompatible housing enclosing one or more of the components.

The electronics module 2100 can include one or more other components to provide stimulation. In many examples, the electronics module 2100 includes one or more components for receiving a signal and converting the signal into the stimulation signal 215. The electronics module 2100 can further include a stimulator unit. The electronics module 2100 can generate or control delivery of the stimulation signals 215 to the stimulator assembly 230 to stimulate tissue proximate the stimulation assembly 2300. In examples, the electronics module 2100 includes one or more processors (e.g., central processing units) coupled to memory components (e.g., flash memory) storing instructions that when executed cause performance of an operation described herein. In examples, the electronics module 2100 generates and monitors parameters associated with generating and delivering the stimulus (e.g., output voltage, output current, or line impedance). In examples, the electronics module 2100 generates a telemetry signal (e.g., a data signal) that includes telemetry data. The electronics module 2100 can send the telemetry signal to the first device 2020 or store the telemetry signal in memory for later use or retrieval.

The apparatus 2000 can include one or more stimulator assemblies 2300 can be one or more components configured to provide stimulation to target tissue. In the illustrated example, there are two stimulator assemblies 2300 with one corresponding to the implantable inhibition assembly 1140 and the implantable stimulation assembly 1240. Further in the illustrated example, the stimulator assemblies 2300 are electrode assemblies that includes arrays of electrodes 2320 disposed on a lead configured to be inserted into the recipient's cochlea. The stimulator assembly 2300 can be configured to deliver stimulation signals 2150 (e.g., electrical stimulation signals) generated by the electronics module 2100 to the cochlea to cause a hearing percept in the recipient. In some examples, the stimulator assembly 2300 is a vibratory actuator disposed inside or outside of a housing of the second device 2500 and configured to generate vibrations. The vibratory actuator receives the stimulation signals 2150 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion. In addition or instead, the actuator can deliver the vibrations to cause tactile percepts in the recipient.

The transceivers 2460 can be components configured to transcutaneously receive or transmit a signal 2510 (e.g., a power signal or a data signal). The transceiver 2460 can be a collection of one or more components that form part of a transcutaneous energy or data transfer system to transfer the signal 251 between the first device 202 and the second device 250. Various types of signal transfer, such as electromagnetic, capacitive, and inductive transfer, can be used to usably receive or transmit the signal 2510. The transceiver 2460 can include or be electrically connected to the coil 2560.

The coils 2560 can be components configured to receive or transmit a signal 2510, typically via an inductive arrangement formed by multiple turns of wire. In examples, in addition to or instead of a coil, other arrangements can be used, such as an antenna or capacitive plates. Magnets 2340 can be used to align respective coils 2560 of the first device 2020 and the second device 2500. For example, the coil 2560 of the second device 2500 can be disposed in relation to (e.g., in a coaxial relationship) with a magnet 2340 to facilitate orienting the coil 2560 in relation to the coil 256 of the first device 2020 via a magnetic connection 2350. The coil 256 of the first device 2020 can also be disposed in relation to (e.g., in a coaxial relationship with) a magnet 2340.

The power source 2480 of the respective devices can be configured to provide operational power to other components. The power sources 2480 can be or include one or more rechargeable batteries. Power for the batteries can be received from a source and stored in the battery. The power can then be distributed to the other components of the second device 2500 as needed for operation.

As should be appreciated, while particular components are described in conjunction with this, technology disclosed herein can be applied in any of a variety of circumstances. The above discussion is not meant to suggest that the disclosed techniques are only suitable for implementation within systems akin to that illustrated in and described with respect to the figure. In general, additional configurations can be used to practice the methods and systems herein and/or some aspects described can be excluded without departing from the methods and systems disclosed herein. For example, while FIG. 2 illustrates a second device 250 being implanted beneath the recipient's tissue, the system 2000 can be formed without an implanted component. Instead, for example, the stimulation assemblies 2300 can be configured to be used externally/external stimulators can be used.

The various components of the system 1000 can cooperate to compensate for a balance dysfunction of a recipient of the system 1000. An exemplary process will now be described.

The process can include obtaining data from the one or more sensors 2420. For example, one or both of vestibular inhibitor 1100 (e.g., the vestibular inhibitor stimulator generator 1120 thereof) and the stimulator 1200 (e.g., the balance signal generator 1220 thereof) can obtain the data. The one or more sensors 2420 can be one or more balance sensors that obtain data relating to balance data. Such data can include, for example, accelerometer data, gyroscope data, or magnetometer data. That data can describe rotation around one or more axes, such as pitch, yaw, or roll axes. Obtaining the data from one or more sensors 2420 can include obtaining data from physiological sensors, such as heartbeat, galvanic skin response sensors, electromyography sensors, or other sensors. In some examples, one or more of the sensors 2420 are disposed remote from the component obtaining the data. The obtaining can include wirelessly obtaining the data from a remote sensor 2420. For instance, in an example, the balance signal generator 122 obtains the data from the commuting device 1300. The process can include inhibiting the recipient's vestibular system. The inhibiting can include the vestibular inhibitor signal generator 1120 generating a signal that causes the inhibition assembly 1140 to stimulate the recipient's vestibular system in a manner that inhibits dysfunctional signals supplied by the recipient's vestibular system. In various implementations inhibiting can be substantially constant, intermittent, performed in response to a schedule, or performed based on the sensor data obtained in operation 3200. The inhibiting can be controlled automatically or manually. For example, a user interface (e.g., a switch, button, touch screen, or wirelessly connected control) can be provided (e.g., at the computing device 1300) to permit the recipient or a caregiver thereof to engage or disengage the inhibition. Such a user interface can also be used to modify an intensity or other parameters of the inhibition being provided.

In some examples, inhibiting the vestibular system can include deactivating tissue associated with the vestibular system, such as by ablating tissue associated with the vestibular system. In some examples, a pharmacological agent is provided to the recipient that inhibits the vestibular system or a perception of signals provided by the vestibular system.

The process can include generating inhibition stimulation signals. The inhibition stimulation signals can be generated using, for example, a processor 2440 or an electronics module 2100 associated with the inhibitor 1100. The generation of the signals can cause the inhibiting to be substantially constant, intermittent, performed in response to a schedule, or performed based on the sensor data. The inhibition stimulation signals can be signals usable to control the delivery of stimulation. For example, the inhibiting can include electrically stimulating the vestibular system with one or more electrodes of the inhibition assembly 1140. The stimulation can be configured to mask naturally-occurring signals generated by the vestibular system that can cause abnormal vestibular percepts in the recipient. In some examples, the inhibiting can include delivering stimulation at approximately 500 Hz, approximately 900 HZ, or at less than 1 KHz.

The process can include applying inhibition stimulation based on the inhibition. stimulation signals. Techniques for applying the stimulation can vary depending on the configuration of the stimulator assembly 2300 being used. For example, where the stimulator assembly 2300 is an electrode assembly, applying the stimulation can include electrically stimulating the recipient using the stimulator assembly. The stimulation can be delivered to an otolith region, semicircular canals, or other regions of the vestibular system of the recipient to inhibit the vestibular system. In another example, the stimulation is delivered to a vestibular nerve.

The process can include ceasing inhibiting the vestibular system. For instance, electrical or other stimulation of the vestibular system can be stopped. The ceasing can be performed in response to any of a variety of events, such as detecting that the recipient is not walking or otherwise moving. For example, it can be desirable to inhibit the vestibular system while the recipient is moving around and to cease the inhibiting at other times (e.g., when the recipient is sitting or lying down). In some examples, the inhibiting is ceased when the recipient is sleeping (e.g., which can be detected based on a variety of factors, such as a time of day, movement of the recipient, a lack of light detected by a light sensor, other factors, or combinations thereof). In some examples, the inhibiting can occur responsive to detecting that the recipient has an abnormal gait or is falling or about to fall. The inhibiting can cease responsive to determining that such events (e.g., a heightened risk of falling) are no longer occurring. And note that any disclosure herein of ceasing inhibition and/or starting inhibition (or suppression) corresponds to an alternate disclosure of ceasing stimulation and/or starting stimulation vis-à-vis embodiments that utilize stimulation for enhancement purposes and/or generation of signals for the brain purposes.

The process can include providing balance compensation output signals to the recipient via one or more non-vestibular sensory channels of the recipient. The providing can include providing first balance compensation output signal while inhibiting the recipient's vestibular system. The providing can include providing second balance compensation output signal while the inhibiting is ceased. The process can include generating one or more balance compensation output signals. The balance compensation output signals can be configured for use in compensation of a vestibular deficiency of the recipient. The process can include obtaining balance compensation input signals 2430 from one or more sensors 2420. Such balance compensation input signals 2430 can include, for example signals relating to rotation about one or more axes.

The process can include generating the one or more balance compensation output signals based on balance compensation input. The process can include encoding data regarding rotation about one or more axes using one or more characteristics. The process can include encoding data regarding rotation about first, second, and third axes using respective first, second, and third characteristics. In some examples, the axes are selected from a group consisting of a yaw axis, a roll axis, and a pitch axis. The axes can be with respect to the recipient, such that the rotation about the particular axis provides information about movement of, for example, the recipient's head. The rotation about a first axis can be determined based on, for example compensation input signals obtained from the one or more sensors 2420. The characteristics can be characteristics of a percept that is ultimately perceived by a recipient. The encoding can include modifying a signal (e.g., the balance compensation output signals) such that the signal ultimately causes a percept to be detected by the recipient having the characteristic. The characteristics can vary based on a stimulation modality (e.g., tactile precept, audio percept, or visual percept). Further, the chosen stimulation modality itself can be a characteristic that can be used to convey balance information. For instance, where the stimulation modality is audio, such audio characteristics that can be varied to indicate rotation about the various axes can include: loudness, pitch, stimulation frequency, melody, rhythm, location (e.g., left or right side), stereo effect (e.g., a relative loudness or other difference between playback on left or right sides), other characteristics, or combinations thereof. Further, the same characteristic can be used to indicate information regarding rotation about multiple axes.

In an example, rotation about first and second axes is encoded using pitch and encoding an extent of the rotation about the axes using volume. For instance, as a recipient rotates their head about a roll axis, a tone having a first pitch can be played at a first volume. As the recipient continues to rotate their head further, the first volume can increase while the pitch remains the same. In addition, as the recipient rotates their head about a pitch axis, a tone having a second pitch can be played at a second volume. As the recipient continues to rotate their head further, the second volume can increase while the second pitch remains the same. The two tones can be played substantially simultaneously to each other. In some examples, negative or positive rotation angles can be encoded based on which side of a head the sound is played. The process can include applying stimulation based on the balance compensation output signals. Applying the stimulation can include generating electrical, vibratory, visual, or other kinds of stimulation based on the signal, such as is described herein. Such stimulation can be configured to provide balance compensation.

The process can include causing a hearing percept. Causing a hearing percept can include stimulating the recipient's auditory system so the recipient perceives an audio event. The process can include electrically stimulating a cochlea of the recipient. For example, the cochlear can be stimulated with one or more intracochlear electrodes. An example of a cochlear implant with which hearing percepts can be caused is described in FIG. 4. The process can include applying vibratory stimulation. The vibratory stimulation can include, for example, causing bone-conducted or air-conducted vibrations, such as from a bone conduction apparatus or consumer audio product, respectively. Such vibrations can cause an auditory precept to be experienced by the recipient.

The process can include causing a visual percept. Causing a visual percept can include stimulating the recipient's visual system so that the recipient perceives a visual event. The process can include activating LEDs (Light Emitting Diodes) or an LCD (Liquid Crystal Display) to cause the visual percept. The process can include directly stimulating a recipient's visual sensory system via electrical or other stimulation.

The process can include causing a tactile percept. Causing a tactile percept can include causing one or more vibratory actuators to vibrate the recipient's skin to tactilely convey balance information.

It is noted that various devices, systems, and methods have been described up to this point which can individually and/or collectively utilized to compensate for a balance/coordination dysfunction or otherwise compensate for a vestibular dysfunction. Embodiments can utilize one or more or all of the teachings detailed herein separately and/or in combination in any manner that can enable the teachings herein. And it is noted that the various devices described above have features that are related to each other in some instances. Accordingly, embodiments include any one or more of the above teachings associated with the device detailed above combined with or otherwise substituted for any one or more of the other teachings of the devices detailed above unless otherwise noted, providing that the art enables such. In this regard, for the purposes of textual economy, some features and/or components and/or method actions associated with one type of device has not been presented because such features can be present in some instances in the other device.

In a normally functioning sensory system of a human being, there can be three sensory inputs that are utilized by the human body to achieve balance. First, there is the vestibular system input, which can provide equilibrium, spatial awareness, rotation and linear moment input. Second, there is also the visual input, which entails sight. The third sensory input is the proprioceptive, which entails touch and feel. In a normally functioning human being, these three sensory inputs are provided to the cerebellum, which coordinates, regulates posture movement and balance. These three inputs can also be provided to the brainstem, which integrates and sorts the sensory information.

Embodiments disclosed herein focus for the most part on the first sensory input, the vestibular. But it is noted that embodiments can also include influencing the visual and/or the proprioceptive. Embodiments can be directed towards enhancing and/or suppressing or otherwise influencing the vestibular system so as to improve the balance and coordination of the human. Again, as noted above, there can be a system that re-creates a feeling of balance by providing stimulation with the signal, there can be a system that inhibits or suppresses the balance function, and there can be a system that produces some other stimulus, such as an acoustic stimulus, that is related information gathered with a sensor system, which can include an accelerometer and/or a gyroscope. Embodiments can include a combination of two or more or all of the systems.

The inventors have determined that there can be utilitarian value with respect to controlling the various systems for providing stimulation to the human to improve balance or otherwise the systems that influence the vestibular function/system based on a state of an environment around the human. By way of example, with respect to the medical device detailed above that suppresses the vestibular balance function, once such a device starts to so suppress, the recipient of that device will need to use other sensory inputs for balance and coordination, such as, for example, the visual system. The present inventors have also determined that in such scenarios, depending on the environment, there can be utilitarian value with respect to increasing or otherwise initiating the provision of sensory substitution relative to that which might otherwise be the case. In this regard, in an exemplary embodiment, by way of example only, embodiments can include evaluating an ambient light level to serve as a basis for whether or not or otherwise how much the vestibular balance function should be suppressed. For example, the visual sensory input functions best when there is a certain amount of ambient light. Too little light, or, in some scenarios, too much light, will decrease the utilitarian value of the visual sensory input relative to that which would otherwise be the case. Accordingly, in at least some exemplary scenarios, by way of example, there could be a detrimental effect to suppressing the vestibular balance function in low light levels. Thus, embodiments include a functionality of the medical device that enables the medical device to receive input indicative of an ambient environment, such as the ambient light level. By way of example only and not by way of limitation, the medical devices detailed herein can include a light capture device, such as a photodiode or a CCD, that can capture light. A light sensor can be used. A photoresistor can be used. Any combination of these can be used providing that the art enables such. In an embodiment, the light capture devices approximate the human eye response. In an embodiment, the medical device includes a processor that can evaluate a signal based on the captured light, and determine or otherwise estimate an ambient light level. In an embodiment, there can be a device that is the same as or otherwise analogous to a device that can automatically determine whether or not to utilize a flash for a digital camera, by way of example. The control logic and/or the settings thereof can be utilized in/with the medical device. Indeed, in an exemplary embodiment, the medical device can be in signal communication with a smart phone, such as that which is the case in FIG. 3A above, which smart phone has a “camera phone,” and has an application thereon that can evaluate a signal from the light capture device, such as the CCD of the smart phone, and determine or otherwise estimate an ambient light level. The smart phone can provide a signal to the vestibular implants or other medical device, such as in a wireless manner such as by a Bluetooth, which indicates the ambient light level, or pending fact provide the ultimate control signal that determines whether or not the medical device should suppress the vestibular balance function and/or by how much, all by way of example.

Note also that concomitant with the cross-pollination of the technologies described herein, as with using the technologies of a cochlear implant for a vestibular stimulation device, embodiments can include utilizing the light capture apparatuses and circuitry (at least some of such) of the retinal implant as the light capture apparatus of the balance sensory devices in some embodiments.

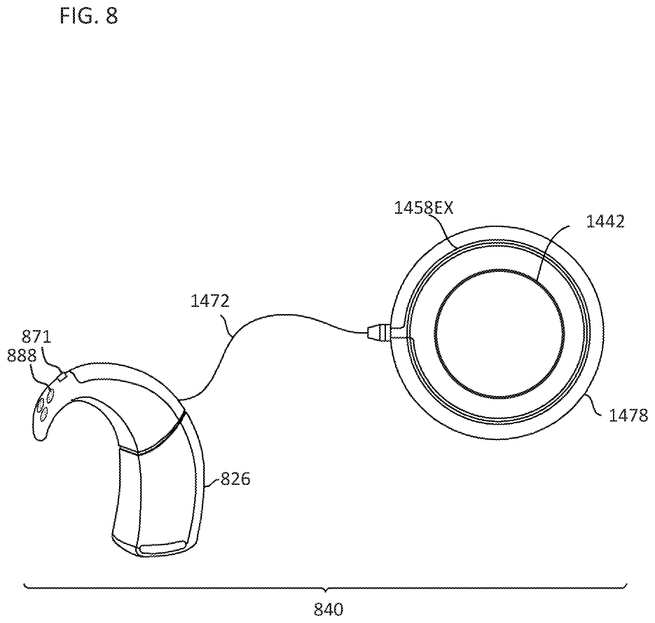

Accordingly, in one embodiment, there is a medical device, such as a vestibular implant, that includes a light capture device on the external device (the BTE device, for example, or an off-the-ear device, for example) that can capture ambient light. The device can measure the ambient light level. In an embodiment, the vestibular implant only starts to stimulate, and inhibit the vestibular function, if the vestibular implant determines that there is enough ambient light in the environment of the recipient. Thus, with reference to FIG. 8, there can be an external component 840 of a vestibular implant that includes a behind-the-ear (BTE) device 826 which is connected via cable 1472 to an exemplary headpiece 1478 including an external inductance coil 1458EX, corresponding to the external coil of FIG. 5. As seen, there are three (3) CCD sensors 888. In an embodiment, elements 888 can be lenses of a smart phone, such as a wide angle lens, or any other lens that can enable the teachings detailed herein. Any commercial off-the-shelf light capture device, that can capture light or otherwise sense light and output a signal, electrical or fiber-optic or otherwise, that can be used adequately evaluate an ambient light level can be used in some embodiments. And in this regard, the signal that is outputted by the light capture device is provided to a computer chip or a processor or some other arrangement of electronics, such as a logic circuit, that receives the signal and analyzes the signal to evaluate the ambient light level based on the signal. The electronics can have circuitry configured to determine or otherwise evaluate the magnitude of the light and/or frequencies of the light (it could be that the wavelengths of the ambient light are useful in determining whether or not to implement the vestibular balance function suppression or otherwise what at level such should be operated). The circuitry could include high-pass or low passband filters.

The external component (implantable component-some embodiments can be directed towards a so-called totally implantable vestibular implant, analogous to a totally implantable cochlear implant) can include circuitry, such as a processor or a computer chip, that can make an evaluation based on the received signal from the light capture device of the ambient light level. More particularly, the external component (implantable component-hereinafter, any disclosure of the external component corresponds to an alternate disclosure of the implantable component, and vice versa, unless otherwise noted, providing that the art enables such) can include a lookup table with pre-stored values. The values of the lookup table can be compared to the pertinent values associated with the received signal from the light capture device by the electronics of the external component. The values lookup table can correspond to light levels that are indicative of good or bad light levels, or otherwise indicative of light levels where the vestibular balance function should be suppressed (or not suppressed). These values can be based on empirical and/or analytical values. All of this can be executed in an automated fashion.

It is briefly noted that the vestibular implant can be configured with a switch or otherwise with and input suite that can enable a recipient to activate and deactivate the light level feature of this exemplary embodiment. In this regard, it could be that the recipient knows that the ambient light level is sufficiently high for the other teachings detailed herein to be implemented, but for some reason or another, the sensors 888 cannot capture adequately the true ambient light. This could be because the recipient is wearing a wide-brimmed hat or for some reason has hair that is extending in front of the sensors 888. That is, embodiments can include the ability to override or otherwise disable the light capture feature of the implant, so that the implant functions as it normally would in the absence of the innovative features associated with the ambient environment sensor.

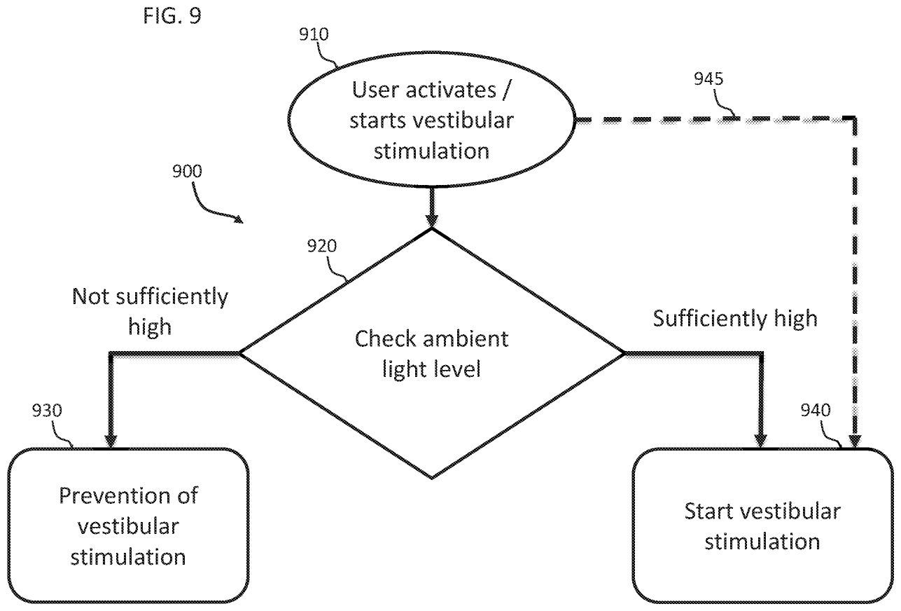

FIG. 9 presents an exemplary flowchart for a high-level exemplary method according to an exemplary embodiment. The method begins at method action 910, where the user initiates vestibular stimulation by activating the vestibular stimulation function on his or her vestibular stimulation implant. In this exemplary embodiment, the vestibular stimulation implant is already implanted, and is otherwise powered up and waiting to be implemented. Prior to method action 910, the implant is turned off, or at least the implant is in a sleep mode or a mode where the implant is not stimulating the vestibular system of the recipient. In this embodiment, even though the user activates the vestibular stimulation functionality of the implant, the device does not start stimulation. Instead, the device moves to method action 920, where the device automatically checks the ambient light level. In this regard, this can be considered a precautionary measure or a potential override measure with respect to the actions of the user/recipient. This can be akin to a lane maintaining functionality of an advanced car. Corollary to this is that in at least some exemplary embodiments, the user/recipient can override this feature, whether before method action 920 or after method action 920. Indeed, in an exemplary embodiment, the vestibular implant is configured so that the logic circuitry bypasses method action 920 and goes directly to method action 940, the features of that method action being described below. Still, with respect to method action 920, here, logic circuitry of the device, whether part of the external portion of the implanted portion, evaluates the signals from the light capture device 888 to check the ambient light level. If a determination is made that the light level is not sufficiently high, the algorithm proceeds to method action 930, which is the prevention of vestibular stimulation by the device. Conversely, if a determination is made that the light level is sufficiently high, the algorithm proceeds to method action 940, which is the start/implementation of vestibular stimulation.

As referenced above, the user can override the environmental sensor features of the implant. This is represented by dashed line 945, which extends from the action of the user activating vestibular stimulation to action 940, which entails the start of vestibular stimulation. In an exemplary embodiment, a dedicated switch 871 can be provided on device 840, and the recipient can depress the switch to activate and/or deactivate the environmental sensor functionality of the implant. The switch 871 can be configured to open or close electrical circuit, which circuit leads to the logic circuitry of the system so as to enable and/or disable the environmental sensor functionality.

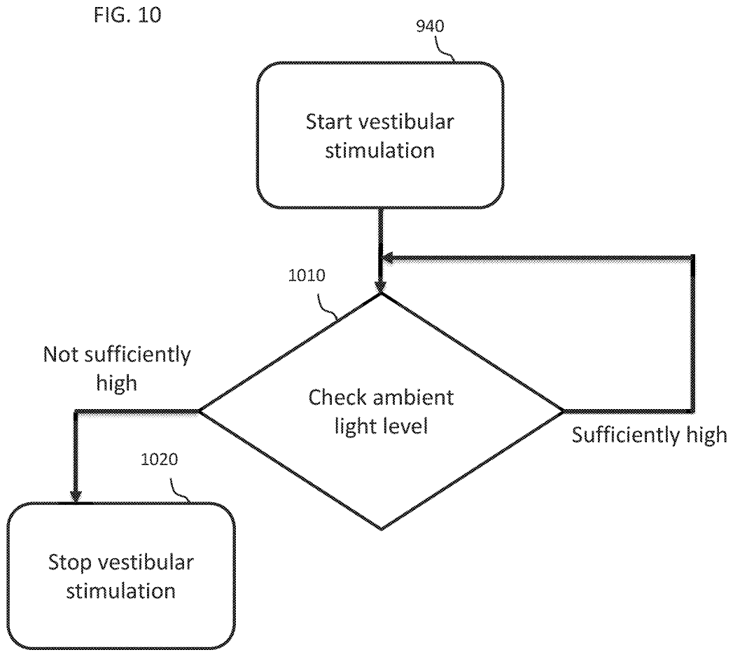

FIG. 10 shows an extension of the algorithm of FIG. 9. Here, upon a determination that the vestibular stimulation should be started at action 940, the vestibular implant periodically, including continuously, checks the ambient light level. Upon a determination that the ambient light level is sufficiently high, there is an essence a continuous do loop. Conversely, upon a determination that the light level is not sufficiently high, the method proceeds to method action 1020, with the vestibular stimulation is stopped. Accordingly, the method represented by FIG. 10 is a method that operates or otherwise is executed while the vestibular stimulation implant is stimulating the vestibular system or at least is enabled to stimulate the vestibular system, because there was a previous determination that the ambient light was sufficiently high, but repeatedly checks the ambient light level, and upon a determination that the light level is not sufficiently high, essentially overrides the functionality. It is noted that in an exemplary embodiment, an indication can be provided to the recipient that the implant is operating according to any one or more of the scenarios. For example, if the implant is operating to provide vestibular stimulation because there is sufficient light, the implant can provide some form of notification to the recipient, whether by an audio chirp or a verbal sound for that matter, or a light or some form of tactile output, and conversely, if the implant stops providing vestibular stimulation because there is insufficient light, (or does not provide such in the first instance), the implant also provides some form of notification to the recipient.

The embodiment of FIGS. 9 and 10 utilizes a binary logic system where either the light level is sufficiently high or not sufficiently high. There is no in between in this exemplary embodiment. Moreover, the functionality is applied, at least with respect to the ambient environment, as an all or nothing functionality in that the vestibular stimulation is started/activated, or the vestibular stimulation is prevented. As will be detailed below, in alternate embodiments, the light level was evaluated on a sliding scale, and the functionality of the implant is varied based on the different levels of light that are sensed slashed can be sensed.

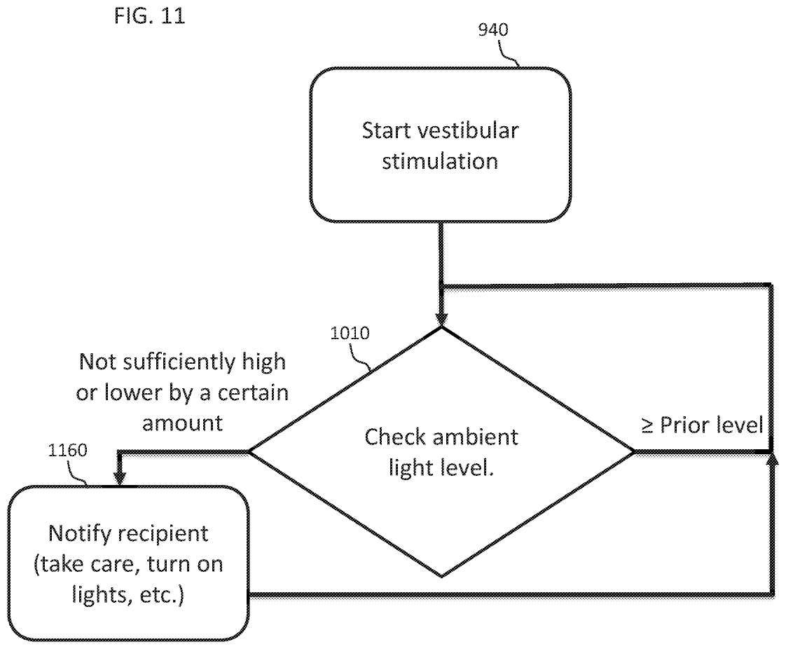

FIG. 11 presents another exemplary flowchart, which again starts at method action 940. Here, the system again executes method action 1010. If the light level is the same as it was before, or greater (for example-again, there could be scenarios where too much light causes a problem, and thus, the check can be a check for light within a range, where below or above is a cause to stop stimulation, or as is the case here, at least notify the recipient to take care), the system continues to provide vestibular stimulation. And upon a determination that the light level has changed so that it is not sufficiently high, or at least lower by a certain amount (which still may result in a light level that is sufficiently high, light represents a reduction in light level that warrants some form of action) the implant proceeds to method action 1160, which entails the implant notifying the recipient of the change in light level, and/or instructing the recipient to take an action, such as at least be careful, or turn on lights, etc. In this exemplary algorithm, after notification, the method goes back to returning to check the ambient light level. If the light level does not change, or more accurately, if the light level does not decrease further, the system can remain in the continuous loop, and if not, the system can re-notify the recipient that the light level has further decreased. Also, in an exemplary embodiment, while the light level is that the reduced light level, the implant can periodically remind the recipient of such or otherwise periodically instruct the recipient to take certain actions. The recipient can override this repeated warning or otherwise cancel this repeated wording by providing input into the external device, such as by depressing the switch 871 by way of example.

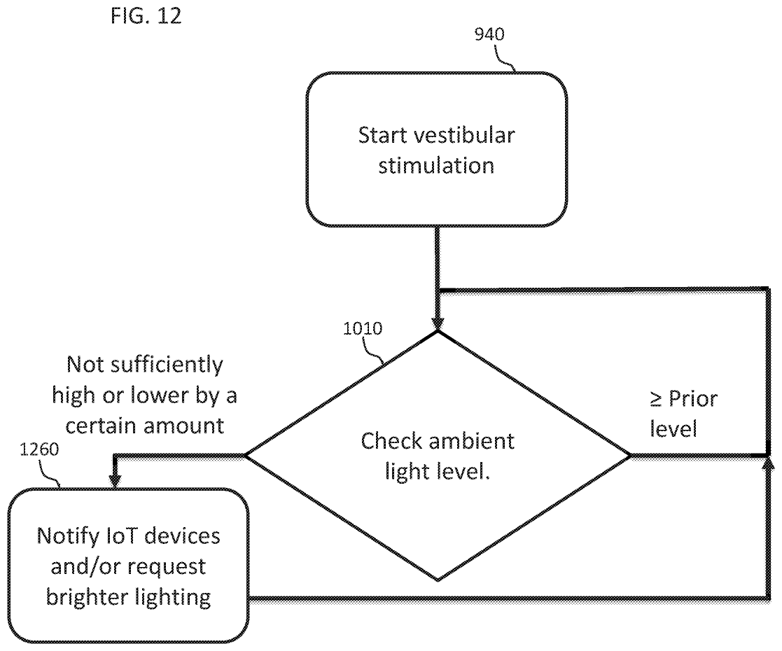

FIG. 12 presents another exemplary flowchart, which again starts at method action 940. Here, the system again executes method action 1010. If the light level is the same as it was before, or greater, the system continues to provide vestibular stimulation. And upon a determination that the light level has changed so that it is not sufficiently high, or at least lower by a certain amount, the implant proceeds to method action 1260, which entails the implant notifying an overall system, such as the Internet of Things and/or specific devices thereof to take an action. In this embodiment, by way of example, the action is to automatically make an adjustment to the state of lights in the area around the recipient. For example, the implant could be in signal communication with one or more devices of the Internet of Things by Bluetooth communication or some other wireless communication regime, and could convey a command signal to the Internet of Things to turn on one or more lights that are currently turned off, or increase the output of a light or a plurality of lights that are currently one but at a low level of output. Alternatively, the output could simply be the value of the light level determined by the implant, or even the raw data from the light capture device, and then the Internet of Things can make the determination as to whether or not or how to adjust the ambient lighting. This process could be iterative in at least some exemplary embodiments. And note while this embodiment focuses on brightening the ambient lighting, consistent with the teachings above, in some embodiments, there can be a scenario where the lighting is actually reduced if the light level increases beyond the upper limits of a given range of values for the light level.

And while the embodiments herein focus on light level, in some embodiments, it can be other features the ambient environment, such as noise level or even temperature for that matter. Any ambient environment feature that can improve the efficacy of the teachings detailed herein can be utilized in some embodiments. Moreover, while the above has focused on light level, again, as briefly noted above, it can be the frequency of light that can be adjusted or otherwise controlled to enhance the efficacy of the teachings herein.

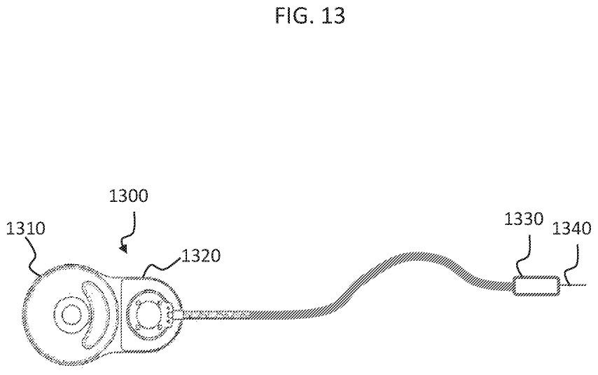

In view of the above, it can be seen that in an embodiment, there is a system, such as a medical system, that comprises a first subsystem configured to neurologically affect a human when activated and a second subsystem configured to obtain data based on an ambient environment of the system. In this embodiment, the system is configured to control the first subsystem, at least in part, based on the obtained data and the system is a human balance medical system. Concomitant with the teachings above, the first subsystem can be a vestibular neurological system stimulation device. The first subsystem can provide electrical signals to the vestibular neurological system of the human, directly or indirectly (again, embodiments can, in some instances, be a totally external device without any implant component, where the electrodes of the system are located supercutaneously for example). The first subsystem can be a therapeutic substance delivery system, such as a device that releases chemicals into the body of the human. This can be a subcutaneously implanted device, that can have, for example, a conduit that is in fluid communication with the vestibular duct(s) and can inject a fluid therein, such as a chemical, that can neurologically affect the human. FIG. 13 presents an exemplary implantable component of a vestibular stimulation device, device 1300, that includes a receiver 1310 and logic circuit/control circuit 1320 which is connected by an electrical lead to a therapeutic substance electromechanically actuated syringe 1330 with a termination 1340. Briefly, the device 1300 can correspond in general terms to the implantable component of the cochlear implant and/or vestibular implants detailed above. The implant 1300 can receive control signals from the external component transcutaneously, which control signals are related to the control circuit 1320. This control circuit converts these control signals into electrical signals that are supplied by the electrical lead to the electromechanical syringe 1330. The termination 1340 extends into a vestibular duct or another component of the middle ear. Depending on the signal received from the external component, the electromechanical syringe 1330 can inject a therapeutic substance into the middle ear when it is determined that such can be utilized for utilitarian value with respect to the treatment regimes detailed herein.

Concomitant time with the teachings detailed above, the first subsystem configured to neurologically affect a human when activated can be a traditional vestibular implant that provides or otherwise re-creates a feeling of balance by stimulating with the signal that is related to information gathered by an accelerometer and/or gyroscope. It can also be a device that suppresses or inhibits the operation of the vestibular system, at least in part. It can also be the device detailed above that provides sensory substitution. It can be a combination of any of these. Still, in an embodiment, the first subsystem is a vestibular implant.

Any device and/or system that can neurologically affect a human when activated to treat a balance and/or coordination problem in a human or mammal that can enable the teachings detailed herein can be utilized in some exemplary embodiments.

Concomitant with the teachings above, the second subsystem can be a light capture subsystem. Alternatively, and/or in addition to this, the second subsystem can be a sound sensor subsystem.

In more utilitarian terms, the overall system can be a system configured to varyingly suppress sensory input to a brain of the human. In this regard, in an exemplary embodiment, the system can be configured to suppress and/or inhibit the vestibular balance function. As noted above, some people can be afflicted with an ailment where the vestibular balance function results in the human becoming unbalanced or otherwise uncoordinated relative to that which would otherwise be the case in a properly functioning vestibular system. There can thus be utilitarian value in suppressing and/or inhibiting the vestibular balance function. In some embodiments, this can be achieved by applying electrical signals to the vestibular neurological system. This can entail directly applying electrical signals to the inner ear, or directly applying the electrical signals to another part of the human, such as the brain, to the spine, or some other portion of the human, providing that such can inhibit and/or suppress the vestibular balance function. Of course, as noted above, signals could potentially be provided from outside the body, and thus the signals could be indirectly applied to one or more of the various body components just noted. Accordingly, in at least some exemplary embodiments, the electrical signals are not necessarily applied to the ear system of the human. Any signal application that can have utilitarian value with respect to the teachings detailed herein can be utilized in at least some exemplary embodiments. Note that in some embodiments, the system is configured to varyingly enhance and/or provide sensory input to a brain of the human, such as input from the vestibula and/or recreating the output of the vestibular system entirely (whether from the vestibular system or at a location elsewhere-any stimulation at any location that can provide utilitarian value with respect to balance can be used in at least some embodiments). Similarly, there are apparatuses herein that are configured to enhance and/or provide signals from the vestibula that travel to the brain by stimulation of tissue of the recipient using the electrical signal(s).

With respect to suppressing the sensory input to the brain in the human, some embodiments can include utilizing the first subsystem in a manner that completely shuts down or otherwise renders meaningless the vestibular balance function of the human. With respect to inhibiting the sensory input to the brain, some embodiments can include utilizing the first subsystem in a manner that limits or otherwise reduces the impact of the vestibular balance function on the human. This can occur in varying degrees as will be described in greater detail below. The point here is that not all embodiments always completely eliminate the vestibular balance function or otherwise render such completely void. Embodiments can include simply limiting the effects of such. Briefly, as will be described in greater detail below, variations of light level can be correlated to levels of inhibition (controlled inhibition) of the vestibular balance function.

And in some embodiments, the system is configured to limit a level of effect of the first subsystem on the human based on the obtained data from the second subsystem. In this regard, again, with respect to the scenario where there are various different levels of light, in a bright or otherwise adequately lit environment, the system allow the first subsystem to operate at its maximum capacity or to its full effect, which could be to completely null the vestibular balance function, or could be to limit the vestibular balance function to the maximum extent intended for that subsystem. For example, if the vestibular implant is designed to inhibit the vestibular balance function to a level of 35% relative to the normal function when the first subsystem is operating at its full capacity, the system might instead operate the first subsystem so that the vestibular balance function is inhibited to a level of 50 or 60 or 70%, for example, relative to the normal function. For example, if the vestibular implant is designed to inhibit the vestibular balance function to a level of 0% relative to the normal function (complete nullity) when the first subsystem is operating at its full capacity, the system might instead operate the first subsystem so that the vestibular balance function is inhibited to a level of no more than 10, 15, 20, 25, 30, 35, 40, 45, 50, 55, 60, 65, 70, 75 or 80% or any value or range of values therebetween in 1% increments, for example, relative to the normal function.

The concept is that a cost-benefit analysis can reveal that there may be less utilitarian value in inhibiting or suppressing the vestibular balance function when the environment is a lowlight environment relative to allowing the vestibular balance function to affect the human without inhibition and/or suppression even though that could have deleterious results. The low light level scenario could sufficiently deprive the recipient of sufficient vision sensory input so that it is better to have the deleterious effects of the vestibular balance function impact the human, as that would be less deleterious than the results of inhibiting or suppressing the vestibular balance function in the reduced vision sensory input scenario. Put another way, a cost-benefit analysis would weigh the effects of reducing the vestibular sensory input against the reduced vision sensory input. If there is sufficient reduced vision sensory input, the reduction in vestibular sensory input would be limited or prevented, even though that vestibular sensory input can itself cause the materials results.

Such embodiments can have utilitarian value in ensuring that or otherwise increasing the odds that the vision sensory input is sufficient to supplement the reduced vestibular sensory input. In this regard, determining that there is sufficient light level for the recipient to see or otherwise adjusting the implementation of the first subsystem based on the light level can provide greater results with respect to providing balance and/or coordination therapy to the human relative to that which would otherwise be the case if the first subsystem was operated without regard to the environment in which the human finds himself or herself, here, the light environment.

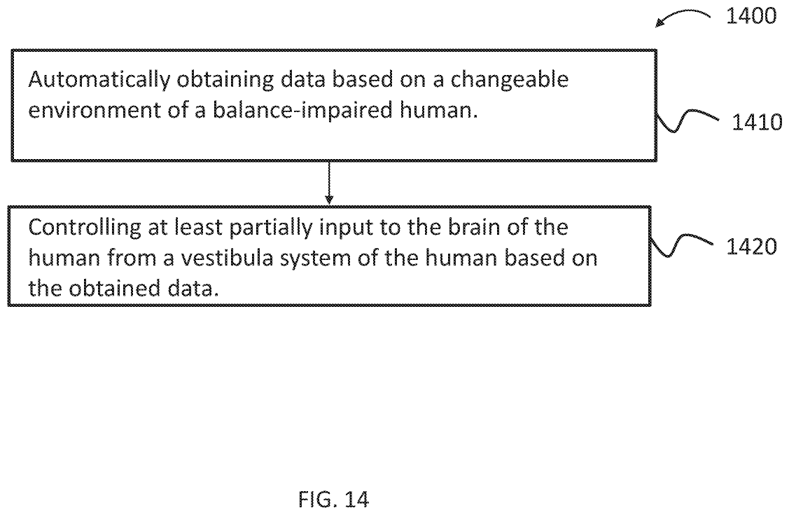

Embodiments include methods. FIG. 14 shows an algorithm for an exemplary method, method 1400, that includes method action 1410, which includes the action of automatically obtaining data based on a changeable environment of a balance-impaired human. This can be the latent environments of the human or the sound environment of the human or a vibrational environment of the human. Any environmental aspect that can have utilitarian value can be utilized in at least some exemplary embodiments. In one of the embodiments, method action 1410 can be executed utilizing the light capture device of the external component of the vestibular system. In another embodiment, alternatively and/or in addition to this, method action 1410 can be executed utilizing a smart phone or the like, where the smart phone can have an application that can activate the smart phones camera or other light capture device of the smart phone (smart phones have a configuration that can adjust the brightness of the display based on the ambient light, embodiments can utilize that to evaluate the ambient light level, and it is noted that some embodiments include an extra component of the implant, such as the BTE device or the off the ear device, that has the hardware and software and firmware and circuitry built into the device (e.g., the light capture device of the smart phone and the logic circuitry thereof that enables the light detection to adjust the screen brightness can be present in the BTE or OTE device)). The embodiment where the light capture device is based on a smart phone, the smart phone can be in signal communication with the implant in accordance with the teachings detailed above. It is noted that in some embodiments, the smart phone can have an accessory that can enable long-range radio field induction so that the smart phone can communicate with the implant without the need of an external component of the implant. Corollary to this is that instead of a smart phone, some other form of portable handheld or body carry device can be utilized that can sense the ambient light level, at which portable handheld or body carry device is not part of the implant system per se, but is an accessory thereto.

Still further, it can be that the Internet of Things or the like can be utilized to provide the data based on a changeable environment of the human. Devices that sense light levels in consumer electronic devices that are located in a household or in the environment where the human lives or works or otherwise spends significant amounts of time, such as inside a car for example, can provide the data or otherwise generate the data that is automatically obtained in method action 1410. By way of example, a video camera of a personal computer can capture light, and the personal computer can have a program thereon that can evaluate the capture light and determine the ambient light level in a room or otherwise in the area proximate the laptop computer. Televisions that have automatic brightness adjustment depending on the light level can be looped into the system to provide the data that is automatically obtained in method action 1410. And while the embodiments above have focused on sensors that capture light, in an alternate embodiment, latent variables can be utilized to assess the light level. By way of example, whether or not one or more or all of the lights in a given room are on or lights of a house or an area/environment are on and/or the power consumption can be utilized to estimate the light level in the environment. Power consumption can be utilized as a latent variable, to determine the settings of lights (a three-way bulb for example). In an embodiment, the Internet of things or otherwise the house or building or infrastructure where the human is located can communicate with the implant or with an accessory device to provide the data based on a changeable environment of the human. The data that is obtained in method action 1410 can be varied and can originate from a variety of sources providing that such can enable method 1400. Indeed, in some embodiments, the action of automatically obtaining data of method action 1410 can be executed with a microphone or the like that is part of the implant are part of an accessory device, with a microphone picks up a voice statement by the balance-impaired human indicative of the brightness. For example, the balance-impaired human could declare that the room is dark or it is not very bright in here, or even make a statement that is not definitive, but indicates an ambient light level, such as it is difficult to see. The microphone could automatically capture the voice and thus execute method action 1410.

And speaking of microphones, again in embodiments where the environment is a sound environment, the microphone of the vestibular implant can pick up sounds in the environment and thus automatically obtain data based on a changeable environment of the human. Vibration sensors can be utilized to automatically obtain the data. Humidity and/or temperature sensors can be utilized to automatically obtain the data, where the data is based on local climate values. Indeed, levels of wind can impact the balance and/or coordination of a balance-impaired human. There could be windspeed and/or wind direction sensors in the system that includes the implant. And again, it is noted that device that automatically obtains the data may not necessarily be a direct part of the implant. It can be an accessory device such as the smart phone.

Note that while the above and the below often focus on the brightness of an environment, the features of the environment that are used for some emblements that relate to the visual input of the human can be broader than just general light levels. Indeed, an environment where there are many moving objects or otherwise objects moving relatively fast or in a non-organized manner could be an environment that has an impact on the vision sense that can affect balance. And in this regard, the actions associated with the environment detailed herein with respect to features that impact the vision sense can be applicable not just to light levels, but to the overall vision “scene” or to a specific part of the vision “scene” that could have an impact on the balance impaired human. Consider a scenario where there are objects moving in different directions, in a chaotic fashion, such as a group of children playing on a playground (or an American soccer game or non-North American football game or a basketball game) or a group of dogs running around in a dog park (consider the Corgis of Elizabeth II running in between the various staff members legs). The environment could be perfectly illuminated, the greatest possible illumination vis-à-vis seeing one's surroundings (frequency, brightness for the pupil dilation, etc.), but the visual senses could be impacted by these movements in a deleterious manner vis-à-vis balance. And it could be that the movements are more organized or even random and such could also impact the visual senses. For example, if the balance impaired human is exposed to a series of cars all traveling in one direction, this could impact the balance impaired person's sense of balance if such person is relying more extensively on the visual senses than otherwise would be the case, such as if the stimulation device was not suppressing or inhibiting the vestibular balance function. The balance impaired person could have a sensation of leaning towards the direction of movement of the cars when that is not the case. And note that it may not necessarily be associated with a pattern or a lack of pattern of the moving objects. It could be the simple fact that objects are moving as opposed to being stationary that results in the balance impaired person having a more difficult balance experience. Accordingly, the features associated with the visual environment are not limited to the more generalized features of like level and/or frequency.

Corollary to this is that in environments where the objects are less defined relative to one another, a balance impaired person may have more problems with balance then in an environment where the objects are more defined. By way of example, a balance impaired person in a forest or looking at dense foliage may have more of a difficult time with respect to balance than if he or she were looking at more well-defined features that are less natural, such as straight lined objects or sharp boundary objects (nature does not work in straight lines).

Note that the action of obtaining in method 1410 does not require the action of formulating the data that is obtained, although that is not excluded from method action 1410. The data can be formulated by third-party, such as a remote database, and then provided to the implant or the user of the implant or whatever medical device is implementing the teachings detailed herein. Indeed, in an exemplary embodiment, a house or building or infrastructure in which the balance impaired person lives or spends some time in can be “wired” with sensors or devices that capture light, and the light levels can be evaluated or analyzed at a remote location via an Internet connection or the like. This remote location can provide the data or otherwise offer the data so that it is accessible by the implant or the user of the implant. The data could be data that is indicative of the light level, or the data could be command data or instructions to the implant or the recipient of the implant to take action. And the data that is obtained in method action 1410 need not be rigorously developed. In this regard, in an exemplary embodiment, a caregiver or healthcare provider or friend or companion of the balance impaired person could provide information relating to the environment that can be changed, such as declaring that it is dark in a certain room or is less bright in a certain room than another room, etc., or it is getting dark out, and that could be the data obtained in method action 1410.

Method 1400 further includes method action 1420, which includes the action of controlling, at least partially, input into the brain of the human from a vestibula system of the human based on the obtained data. This can entail utilizing any one or more of the devices detailed herein or variations thereof to re-create a feeling of balance by stimulating with a signal that is related to information gathered with an accelerometer or gyroscope or some other position capture device orientation capture device. Thus, the action of controlling at least partially input to the brain includes commencing artificially stimulating a vestibular neurological system of the human. This can be done with a vestibular implant as detailed herein, or some form of the external device that applies an electrical current to the skin of a human providing that it artificially stimulates a vestibular neurological system of the human. This can also be accomplished by providing a chemical as noted above. And note that the chemical that is provided can be provided directly to the inner ear utilizing, by way of example, the device of FIG. 13, or can be provided in a less invasive manner, such as a transdermal lumen or an injection or the like.

This can also be the inhibition or suppression of the vestibular balance function. Thus, in an embodiment, the action of controlling at least partially important to the brain includes limiting vestibular function, which covers both the action of inhibiting and suppressing. And this can also be sensory substitution for example. Any device, system, and/or method that controls at least partially, input into the brain of the human from the vestibula system can be utilized in some embodiments, providing that it is obtained from method action 1410.

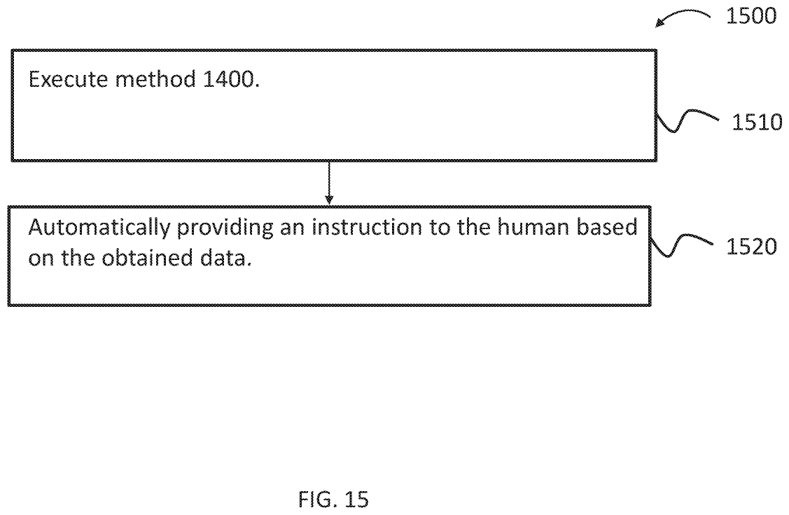

FIG. 15 provides another exemplary flowchart for an exemplary method, method 1500. This method recalls the teachings above regarding the exemplary embodiment where an action is recommended to be taken by the balance impaired person or otherwise the recipient of the prosthesis, which action can be utilitarian vis-à-vis at least lowering a likelihood that the balance impaired person could hurt himself or herself. Accordingly, method 1500 includes method 1510, which entails executing method 1400, and also includes method action 1520 which includes the action of automatically providing instruction to the balance-impaired human based on the data obtained in method action 1410.

Returning back to method action 1420, in an exemplary embodiment, the action of controlling at least partially input to the brain includes halting artificial stimulation of the vestibular system of the human. In this regard, it is noted that there are types of prosthetic devices by inhibiting or suppressing the balance function by stimulation of the neurological system of the human, such as by an unmodulated signal. These types of prosthetic devices must rely more so on the other sensory inputs that feed into balance, such as sight. If it is deemed that the light level is too low so that the sight sense cannot adequately compensate for the diminished vestibular portion of the input, the artificial stimulation of the vestibular system that inhibited or suppress the balance function is halted. The idea here is that even with a deficient vestibular system, the recipient is better off with the full effects thereof then with the more limited vestibular system which then enhances the reliance on the sight input, because the light level is lower than that which would otherwise be sufficiently utilitarian. It is thus to be understood that controlling input into the brain can be executed by halting the provision of the electrical stimulation (as distinguished by not providing electrical stimulation—that alone is not what is covered by the action of controlling in method action 1420—doing nothing is not controlling, but changing what one is doing is controlling). Corollary to this is that method action 1420 can entail controlling at least partially input to the brain by varying artificial stimulation of the vestibular system of the brain. Instead of halting the artificial stimulation, the artificial stimulation could be varied. For example, if the inhibition of the balance function is such that the vestibular balance function is reduced to 50% relative to that which would otherwise be the case in the absence of the inhibition, the artificial stimulation can be varied so that the vestibular balance function is reduced to 75% relative to that which would otherwise be the case or some other value. And note that the action of varying artificial stimulation need not necessarily result in the lessening of the “damping” of the vestibular balance function. If the light level has been deemed to increase or otherwise is deemed to be better than that which was previously the case, the artificial stimulation could be varied so that the inhibition of the balance function is reduced so that the effect of the balance function on the balance impaired human is lower than that which was previously the case. In this regard, if the light level is better than it was before, the more aggressive the stimulation treatment can be to inhibit the balance function.

In another exemplary embodiment, method action 1420 can be executed by commencing artificial stimulation of the vestibular system of the human. This occurs when there was no previous artificial stimulation of the vestibular system. In an exemplary embodiment, if the light level improves, the inhibition and/or suppression actions of the implant or other prosthetic device can begin to be implemented whereas previously they were not being implemented. In an exemplary embodiment, the action of commencing artificial stimulation occurs where there was no artificial stimulation within the past 1, 2, 3, 4, 5, 6, 7, 8, 9, 10, 15, 20, 25, 30, 35, 40, 45, 50, 55, 60, 65, 70, 80, 90, 100, 120, 150, 180, 250, 300, 350, 400, 500, or 600 or more minutes, or any value or range of values therebetween in one minute increments. In an exemplary embodiment, the actions associated with method 1400 and/or 1500 can be executed automatically. Indeed, it is noted that unless otherwise noted, providing that the art such, any action detailed herein can be executed automatically by the prostheses or by a remote device, such as a smart phone, or any other the devices detailed herein, such as a laptop or desktop computer or a remote device remote from the recipient/balance impaired person, providing that the art enables such, unless otherwise noted. And any of the method actions detailed herein can be executed in a manual manner or by a human in a non-automatic matter unless otherwise noted, providing that such can be executed by the human.

Much of the above is focused on the vestibular prosthesis that inhibits or suppresses the vestibular balance function. It is noted that many of the above-noted features of the method can also be practiced by the other types of vestibular prostheses or other balance prostheses. For example, the action of commencing artificial stimulation of the vestibular system of the human can be executed by the type of vestibular implant that re-creates a feeling of balance based on some artificial device, such as an accelerometer and/or gyroscope. Such is also the case with respect to varying the artificial stimulation, and halting the artificial stimulation. Accordingly, any disclosure herein of utilizing a device that inhibits or suppresses the vestibular balance function corresponds to an alternat disclosure where the vestibular system is stimulated by the stimulation device to achieve improved balance and/or improved coordination and/or improved peace of mind of the human. Any disclosure herein of vestibular stimulation by a medical device corresponds to a disclosure of suppression/inhibition and an alternate disclosure of the opposite and/or stimulating to provide a sense of balance and/or to enhance the vestibular balance function.

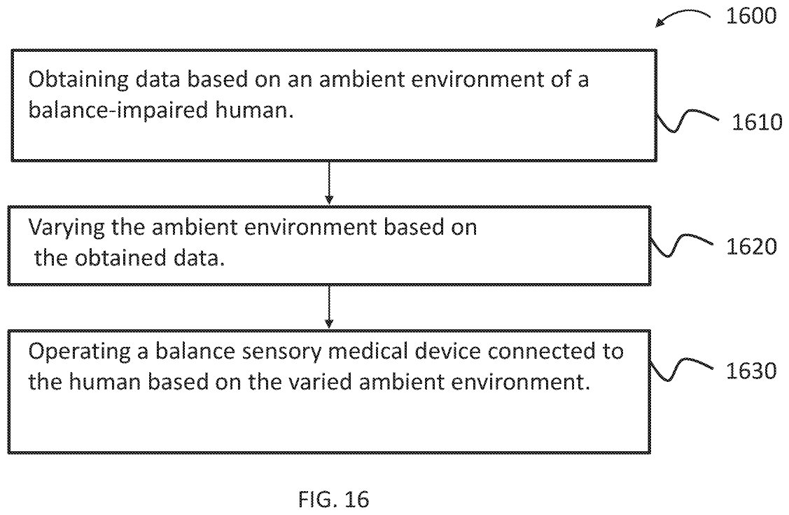

FIG. 16 presents another exemplary algorithm for another exemplary method, method 1600. Here, method 1600 includes method action 1610, which includes the action of obtaining data based on an ambient environment of an impaired human, such as a balance impaired human or a motor function impaired human (for purposes of textual economy, most of the descriptions herein will be directed to a balance impaired human and/or a balance sensory system, but again, as noted above, any disclosure of such corresponds to an alternate disclosure of a motor impaired person, etc., and vis-a-versa, providing that the art enables such). Unlike method action 1410 above, this need not be executed automatically, although it can be, and the corollary to this is that in a variation of the method 1400, method action 1410 in this variation is not executed automatically.

It is briefly noted that the phrase based on an ambient environment includes data that is directly related to a measured or analyzed property, such as the light level, as well as data that is based on data that is directly related to a measured or analyzed property, such as an overall characterization of the measured or analyzed property. For example, data based on an ambient environment could be a qualifier that the ambient environment is bright, which is based on a measurement indicating that the ambient environment at the sensor that was utilized to capture light has Y lumens for example. As long as there is a nexus to the ambient environment, the data is based on the ambient environment.

Method 1600 further includes method action 1620, which includes the action of varying the ambient environment based on the data obtained in method action 1610. Again we return to the scenario where the ambient environment of the impaired human (e.g., balanced impaired human) is such that the light level is low, sufficiently low that it may be inadequate for the impaired human to see his or her surroundings or otherwise process the visual input of his or her surroundings in a sufficiently timely and/or accurate manner that his or her visual senses can provide for compensation for a reduced vestibular function (e.g., reduced vestibular balance function).

Of course, it could be that the ambient environment is so bright that the brightness reduces the efficacy of the visual input vis-à-vis the inputs that are utilized to achieve the ultimate goal of balance and/or improved motor function. In this regard, the phenomenon can be well understood where sunlight is so bright that people sometimes wear sunglasses that actually limit the amount of light reaching one's eyes, but improve the sensations associated with sight relative to that which would otherwise be the case. Corollary to this is the phenomenon where a person walks out of an area that is relatively dark and into an area that is much brighter, such as from a house illuminated by electric lights to the outside in a cloudless and treeless area at midday. A person's eyes will take time to adjust. And in this regard, it is noted that the teachings detailed herein are temporal in nature and that in some embodiments, there can be devices, systems, and methods that are dynamic in nature and account for the fact that the balance impaired person's eyes must take time to adjust to a changed environment, and this works for a change in environment going from dark to bright (relative) and from relative brightness to relative darkness. The balance impaired human's eyes will adjust over time, and thus an ambient environment that may be problematic for the balance impaired human at a first temporal location may not be problematic or otherwise as problematic at a later temporal location. Accordingly, the teachings detailed herein vis-à-vis ambient environments may not be, in some instances, hard and fast requirements or absolute teachings as they can depend on changed circumstances. Thus, embodiments include taking into account the human ability to adapt to a changed environment over time. Accordingly, embodiments include taking into account the temporal nature of a changed environment. In an exemplary embodiment, such as where the ambient light decreases from a first value to a second value, where that second value initially is sufficiently problematic so as to warrant taking some form of action with respect to the implant for example, after a certain amount of time, that second value may be no longer sufficiently problematic that whatever action was taken at the first temporal period can be removed or otherwise relaxed at the second temporal period.

Accordingly, the action of varying the ambient environment may entail initially increasing or decreasing a brightness of the ambient environment, and then more slowly reversing at least in part that increase or decrease (actually decreasing the brightness from an increase brightness are actually increasing the brightness from a decrease brightness, all over time). Thus, where the ambient environment is an ambient light level, the varying of the ambient environment could be reducing the light level or increasing the light level (the latter including turning lights on).

Corollary to the above is that the method actions associated with method 1400 and adjusting a vestibular implant or other balance prostheses based on the obtained data based on the changeable environment of the person can also include readjusting or periodically providing further adjustments to the prostheses are implant to account for the fact that the human's body is adapting to the initial exposure to the environment, even though the environment is no longer changing or otherwise is not changing. And it is noted that a changed environment does not necessarily mean that something happens in the environment. It can be that the balance impaired human has gone from one environment to the other, such as walking from inside a house to outside the house into the bright sunlight (or into the darkness-the human body adapts in both instances over time). Accordingly, embodiments include devices, systems, and methods that have algorithms that account for the human body's adaptations. This can be based on human factors engineering data for a statistically significant population of which the balance impaired person is a part, are based on subjective/individual data relating specifically to observable features of the specific balance impaired person. It is noted that the utilization of such data for setting or adjusting or otherwise refining the implementations of the teachings detailed herein are not limited to body adaptation. Embodiments can include utilizing human factors engineering and/or subjective, specific observable features of the human as a basis to implement one or more of the teachings detailed herein providing that the art enables such.

In any event, regardless of whether the ambient environment is dim or too bright, some form of actions taken to vary the ambient environment. Method 1600 further includes method action 1630, which includes the action of operating a sensory medical device, such as a balance sensory medical device or a motor function medical device connected to the impaired human (balance impaired and/or motor function impaired) based on the varied ambient environment. Here, there can be an automatic determination such as by the prosthesis or the prosthesis accessory/assistant device or by some other device, that the now varied ambient environment is in a sufficient condition to operate the medical device, if only in a certain way. This could be to increase the level of inhibition of the vestibular balance function, or to suppress the vestibular function (e.g., vestibular balance function). This could also be to provide more or less sensory substitution, or provide sensory substitution in the first instance. And this could also be to begin applying stimulus or adjust the stimulus that re-creates the feeling of balance for example or otherwise improves motor function.

Method action 1630 could also be executed manually, such as by activating the inhibition or suppression function or by elevating the suppression function for example.

Again, while embodiments have focused on light, it is noted that the ambient environment can have other features that can impact the overall efficacy of the teachings herein, such as the level of sound of the types of sound in the environment, etc., some sounds can be distracting or otherwise disorienting, and otherwise can have reverberant features that could “trick” the balance impaired person into perceiving a sensation with respect to his or her position and/or balance that is not consistent with reality. Thus, in an embodiment, the ambient environment is sound. Accordingly, the variation of the ambient environment could be reducing a sound level (volume) or changing a frequency of ambient sounds, or changing a direction of origin of sound (to address reverberant sounds, for example).

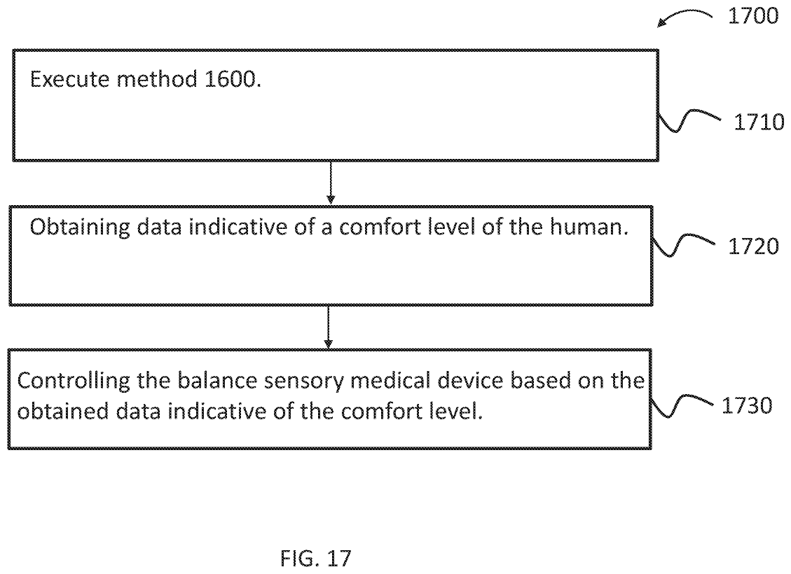

FIG. 17 shows another exemplary flowchart method 1700 for an exemplary method, method 1700, which includes method action 1710, which includes the method action of executing method 1600. Method 1700 further includes method action 1720 which includes the action of obtaining data indicative of a comfort level of the human. The comfort level of the human could the related to the ambient environment's impact on the balance impaired human. A noisy environment could be distracting to the human in a bright or dark environment could also be distracting. Indeed, as noted above, an environment where there are many moving objects or otherwise objects moving relatively fast or in a non-organized manner could be an environment that has an impact on the comfort level of the human. A very bright environment can be uncomfortable.

Embodiments can include devices, systems, and methods that automatically ascertain a comfort level of the human, or at least data indicative of a comfort level of the human. This can be based on latent variables, such as a body temperature or a heart rate or a blood pressure of the human, or how the human is talking (a fast rate of speech could the indicative of agitation, or the mispronunciation of words could be indicative of the person being uncomfortable, etc.). Body sensors can be provided with the prostheses, such as temperature monitors (e.g., an infrared monitor that can detect skin temperature and thus extrapolate from that body temperature), eye-movement monitors, EKG and/or EEG monitors, a microphone can be part of the prosthesis or the microphone to the smart phone or a microphone of an accessory/assistant device or a microphone of the Internet of Things can be utilized to capture speech (which could also be utilized to capture sound in embodiments where the environment that is evaluated is a sound environment). Indeed, visual cameras could be utilized to sense facial features, such as the cameras of the smart phone. All of this can be utilized to obtain data indicative of a comfort level of a human. The various devices herein can be configured with electronics and/or processors and/or computer chips and/or firmware or software or circuitry that can receive the data indicative of a comfort level of a human, such as from the various sensors just detailed, and then analyze that data to obtain an estimation of the current comfort level of the human.

The data indicative of a comfort level of a human can be used in method action 730, which includes the action of controlling the balance sensory medical device based on the obtained data indicative of the comfort level of the human. Here, even if the environmental factors are indicative of something that might be problematic for the balance impaired human, if the balance impaired human is relatively comfortable even in this environment, the balance sensory medical device might be set at a more aggressive level than that which would otherwise be the case. For example, in the case of the vestibular implant that suppresses and/or inhibits the vestibular balance function, the suppression and/or inhibition function might be reengaged because the balance impaired person is comfortable with the environment. Corollary to this is that if the balance impaired person is deemed to be agitated or otherwise uncomfortable or otherwise experiencing symptoms indicative of such, the vestibular implant that suppresses and/or inhibits the vestibular balance function might be deactivated or the aggressiveness thereof might be scaled-back. All of this can be because the comfort level can correlate to distraction or lack of distraction or otherwise the ability of the sensory system of the human to process qualitatively, quantitatively and/or within a sufficient temporal time period the visual input that plays a role in the human balance function. Accordingly, by obtaining data indicative of a comfort level of the human and controlling the balance sensory medical device based on the obtained data indicative of the comfort level of the human, a more efficacious results can be achieved from the use of the balance sensory medical device.

Embodiments include methods where method action 1730 includes adjusting/varying an output of the balance sensory medical device based on the varied ambient environment and operating the balance sensory medical device at the adjusted/varied output. By way of example only and not by way of limitation, an output level of the balance sensory medical device can be increased or decreased. Output levels of the medical device can relate to the stimulation current amplitude, the rate/frequency of the stimulation current, and/or the pulse length/width by way of example only and not by way of limitation. An adjustment of an output level can entail adjusting one or more or all of these features. An adjustment to the output level can entail adjusting the stimulation current but not the rate and not the pulse length. An adjustment to the output level can entail adjusting the pulse rate but not the stimulation current and not the pulse length. An adjustment to the output level can entail adjusting the pulse length but not the other two. An adjustment to the output level can entail two of the three and not the third or all three is just noted above.

Embodiments include empirical analysis of what types of adjustments can have utilitarian value for the given human with respect to various environmental conditions and/or changes. Embodiments can also include the utilization of statistically significant data sets from similarly situated balance impaired people, such as balance impaired people falling within a specific human factors engineering subset that is statistically significant or otherwise statistically pertinent to the human at issue. Accordingly, it could be that for a given individual, it is the stimulation rate that is adjusted given an environmental change or an environmental seen as opposed to stimulation current or pulse length for another individual. It could be stimulation current amplitude that is changed and not the other two, or stimulation current and pulse length but not rate, or rate and stimulation current level but not pulse length, etc. Any of the permutations possible with respect to output being adjusted can be utilized in at least some exemplary embodiments. The idea is that different people may react differently with respect to the output of the balance sensory medical device, at least when correlated to different environmental scenarios, and the teachings detailed herein include operating the balance sensory medical device the in a manner that is customized to the particular balanced impaired human, whether based on strict subjective analysis or statistically significant data for a group of similarly situated individuals, or a combination of the two.

Note that adjusting the output level can include halting stimulation entirely consistent with some of the embodiments above. Any one or more of the exemplary features of the stimulation can be increased or decreased depending on the scenario. For example, in embodiments where there is inadequate lighting, it could be that the stimulation current is reduced, including halted altogether, whereas if there was an increase in the lighting to a more adequate are totally adequate lighting level, the stimulation current could be increased, at least for a balance sensory medical device that is configured to suppress the vestibular balance function. The same could be the case for the rate or the pulse, as well, or a combination of two of the three or all three. Still, with respect to embodiments that focus on the suppression of the vestibular balance function, the output level will often be decreased in a scenario of inadequate lighting.

In an exemplary embodiment, the action of operating the balance sensory medical device includes operating the device in a manner that achieves an increased aggressiveness of a treatment by operating the balance sensory medical device differently from a previous operation based on the varied ambient environment. This increased level of aggressiveness can correspond to operating the device at an increased level of output according to the teachings above. That said, in some scenarios, and increased aggressiveness may be achieved by actually decreasing the output level of one or more of the various variables detailed above. By aggressiveness, it is meant the overall intended results from operating the balance sensory medical device in a certain manner. For example, with respect to the embodiments where the balance sensory medical device is configured to suppress and/or inhibit the vestibular balance function, a more aggressive treatment could be greater inhibition of the vestibular balance function. It could also be a greater temporal suppression of the vestibular balance function. In this regard, it could be that at a given setting, the vestibular balance function is suppressed sometimes but not all the time, and an increased aggressiveness would suppress the vestibular balance function so that the likelihood of a period of non-suppression is lower than that which would otherwise be the case.

In an exemplary embodiment, the increase in aggressiveness of the treatment could be an increase in the results of the re-creation of a feeling of balance and/or can be the increase of a sensory substitution, or more accurately, the results (effect) on the human are increased. Conversely, the action of operating the balance sensory medical device can include operating the device in a manner that achieves a decreased aggressiveness of a treatment by operating the balance sensory medical device differently from a previous operation based on the varied ambient environment. This could entail halting stimulation entirely, or could entail adjusting one or more of the above-noted variables so that the effects of the balance sensory device on the human are reduced relative to that which was previously the case.

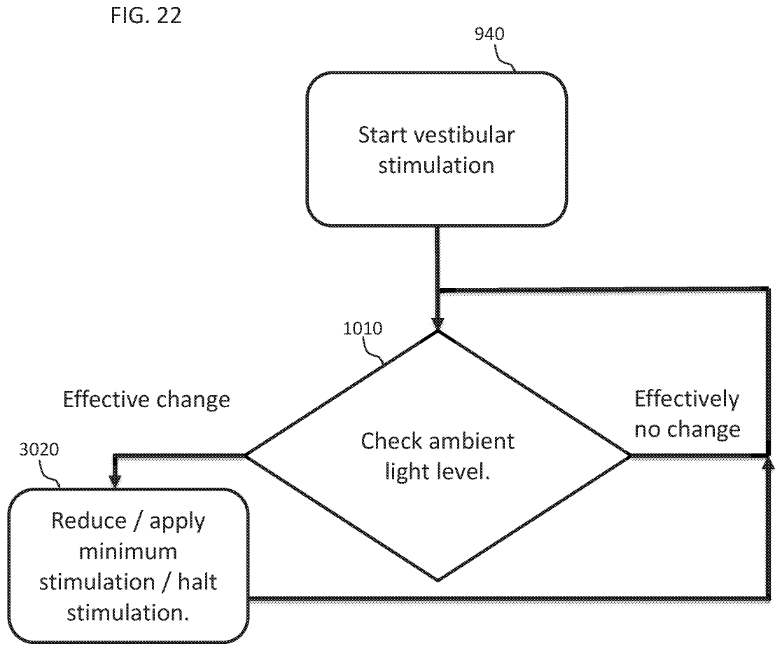

Embodiments above have focused on the concept that there are certain light levels that may be inadequate to operate at least some embodiments of the balance sensory prosthesis relative to other light levels, such as a dark ambient light environment. Embodiments have also focused on the concept of a “too bright” environment and embodiments that briefly focused on the concept of an environment the changes swiftly. Corollary to this is that there could be ambient light environments that are otherwise perfectly fine for the human to continue receiving the therapy for example, but it is the change itself that creates a level of discomfort that warrants a change in the balance therapy. In this regard, the present inventors have found that when an ambient light level changes, at least when it changes by a certain amount, a balanced impaired person can experience balance challenges that here she might otherwise not have experienced had that light level been present previously for a sufficient amount of time. Note that this is not a phenomenon that is based on the fact that eyes take time to adjust to a change in brightness, although that can be a part of this or otherwise can result in this phenomenon, at least in part. This is more a psychological phenomenon that can have impact on balance in general, and can impact on the efficacy of the balance sensory prosthesis/medical device in particular. Accordingly, FIG. 22 presents an exemplary flowchart for an exemplary method, that that includes repeatedly checking the ambient light level. Method action 940 and 1010 are as described above. If there is no change in the ambient light level, the routine continues, but if there is, the routine continues to method action 3020, which entails the action of reducing and/or applying minimum stimulation and/or halting stimulation. Here, if there is a change that is an effective change, were effective means that it has an impact on the human, or is statistically speaking likely to have an impact on the human, whether that is based on empirical results for a specific human or statistically significant data for a class of people in which the human is a part, the balance sensory device will be adjusted or operated differently, In some scenarios or for some types of recipients are particular recipients, the different operation may be to halt all stimulation or reduce stimulation or reply a minimum level of stimulation, or at least reduce the aggression of the treatment to a minimum level as would be known are expected with respect to the given human at issue. In an exemplary embodiment, the change in lighting, whether it is an increase or decrease, could cause balance problems in that the human's visual senses may not be as effective as that which was previously the case before the change, and thus there can be utilitarian value with respect to reducing and/or eliminating the amount of suppression of the vestibular balance function.

In an exemplary embodiment, the method of FIG. 22 proceeds from method action 1010 to method action 4020 upon a change in light level of less than, greater than and/or equal to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 80, 90, 100, 110, 120, 130, 140, 150, 160, 170, 180, 190 or 200 lux. or any value or range of values therebetween in 0.1 lux. increments, and does not proceed if not. In an embodiment, the change takes place within 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 80, 90, 100, 110, 120, 130, 140, 150, 160, 170, 180, 210, 250, 300, 350, 400, 450, 500, 600 or 700 seconds or more or any value or range of values therebetween in 0.1 second increments.

Note also that method action 4020 can also include or in the alternative, includes the action of applying some form of alternate stimulation other than vestibular stimulation, concomitant time with the above-noted prostheses that applies a supplement stimulation and otherwise provides sensory substitution, such as in the form of an acoustic stimulus. Note further that the “and/or” is meaningful in all cases. In some embodiments, method action 4020 includes the action of only applying other stimulation other than vestibular stimulation. That is, there is no vestibular stimulation applied, and the stimulation that is applied is other stimulation. Note further that the application of other stimulation other than vestibular stimulation requires some form of affirmative act beyond that which is the case with respect to normal or ambient stimulation. This could be modifying the normal or ambient stimulation in some manner that would otherwise not be the case in the absence of such modification. This could be the additional input of stimulation, such as noise stimulation, in accordance with the teachings detailed above by way of example only and not by way of limitation.

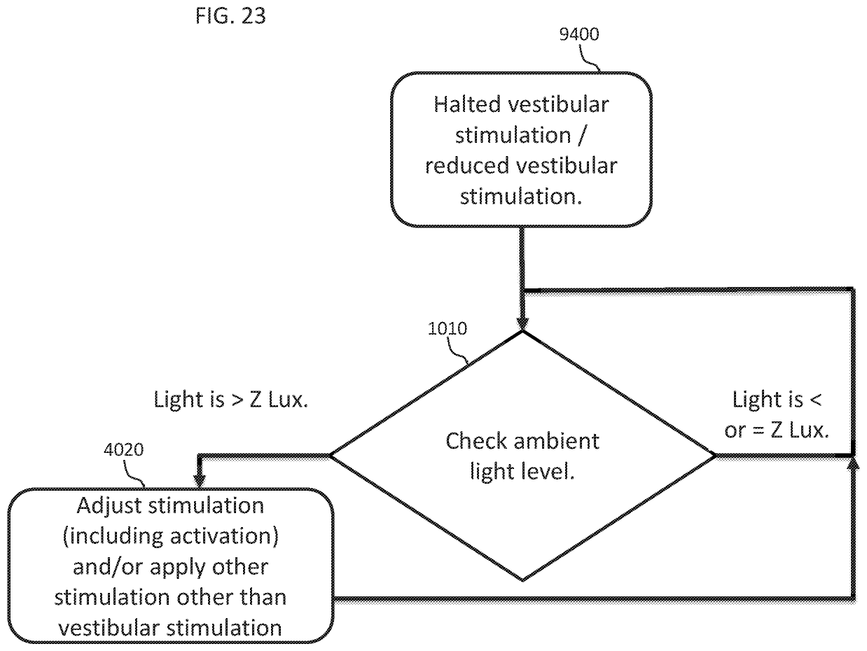

FIG. 23 presents another exemplary algorithm for an exemplary method, again where method action 1010 remain as above, but method 9400 is the halting/reduction in vestibular stimulation (for whatever reason, including that the light level has changed warranting such). Here, there is a qualifier on the ambient light of the value Z lux. Z can be in some embodiments, 30, 31, 32, 33, 34, 35, 40, 45, 50, 55, 60, 65, 70, 80, 90, 100, 110, 120, 130, 140, 150, 160, 170, 180, 190, 200, 225, 250, 275, 300, 350 or 400 lux. or any value or range of values therebetween in 0.1 lux. increments. The value of Z can be selected in preset based on empirical and/or statistical data and/or a combination of the two consistent with the teachings above/herein. It is noted that the values of Z need not be the same for all embodiments. Z is used herein for textual economy. Any use of Z for one embodiment can have a different Z in another embodiment.

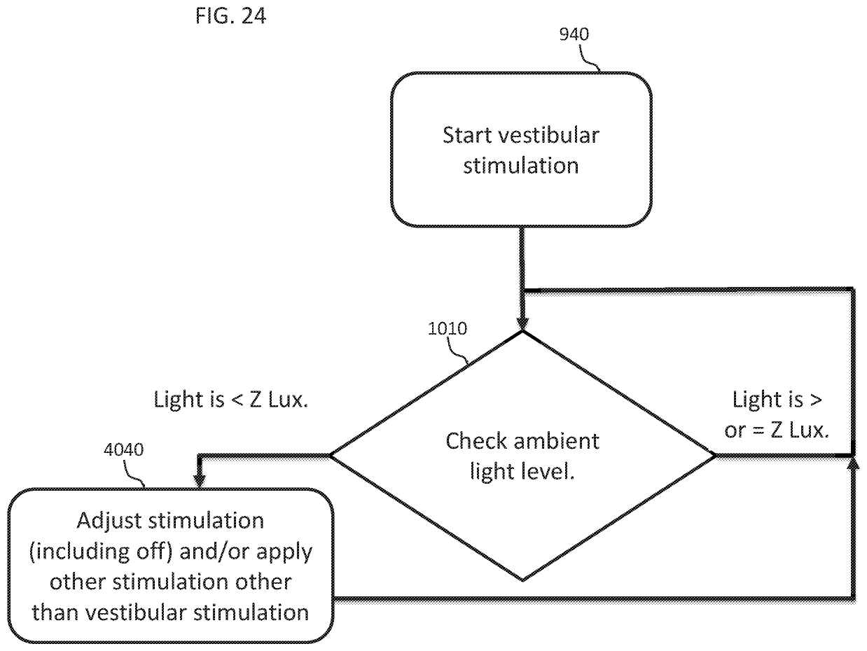

FIG. 24 presents another exemplary flowchart for an exemplary method where method actions 940 and 1010 are as above, and the triggers for the ambient light level can be based on the variable Z as noted above, where method action 4040 can correspond to method action 4020 above, except that the adjustment to stimulation can include turning off stimulation.

The embodiment of FIGS. 23 and/or 24 can be a scenario where the stimulation is adjusted, whether that could be to adjust the stimulation to minimum output power or to actually increase the stimulation, depending on the scenario or the utilitarian value with respect to a given balance impaired person.

Returning back to the apparatus concepts of the innovative features herein, in an exemplary embodiment, there is an apparatus, comprising one or more electrodes, a power source, a light capture device, and a control unit. In an exemplary embodiment, the electrodes could be the implantable electrodes of the vestibular implant, or could be extra cutaneous electrodes or any electrodes that can enable the teachings detailed herein. In an exemplary embodiment, the power source can be a rechargeable battery or could be a capacitor, such as a super capacitor or a plurality of capacitors, or could be a non-rechargeable battery, which could have utilitarian value with respect to an arrangement where the power source is located externally and the electrodes are implanted in the human. The light capture device can be any of the light sensors detailed herein, and can be a photodiode or a sophisticated CCDs (cameras are not light sensors, as a light sensor is a less-sophisticated device). The control unit can be electronics detailed herein and/or a processor or a chip or any microprocessor component that can enable the teachings detailed herein. In an exemplary embodiment, the control unit can be the control unit of the vestibular implant, which can be a commercial off-the-shelf device or could be a modified device, such as by the addition of firmware and/or hardware, such as the addition of a memory chip for a logic chip, etc.

In an exemplary embodiment, the apparatus is configured so that the control unit controls electrical signal(s) to the one or more electrodes to provide balance therapy and/or motor therapy to a recipient of the apparatus, the apparatus also configured so that the control unit controls the electrical signal(s) based on output from the light capture device. Consistent with the teachings above, the apparatus can vary the stimulation or otherwise control the stimulation from the electrodes based on the light level or otherwise the visual scene associated with the environment in which the user of the apparatuses located. In an embodiment where the system provides motor therapy, this can provide stimulation to stop and/or limit and/or control tremors or shaking by way of example. In an embodiment, this can provide refined motor function beyond that which would otherwise be the case. In an embodiment, the teachings herein, such as the applied stimulation, can improve scores on the Peabody developmental scale and/or the Purdue Pegboard Test and/or the Box and Blocks Test and/or Strength-dexterity test by at least 5, 10, 15, 20, 25, 30, 35, 40, 45 or 50% or any value or range of values therebetween in 1% increments relative to that which would otherwise be the case without the teachings herein, all other things being equal.

Embodiments utilizing the stimulation detailed herein can provide improved spatial orientation perception by the recipient. Accordingly, embodiments include spatial orientation perception systems and methods of improving spatial orientation and spatial orientation perception devices, such as those that rely on stimulating tissue.

In an exemplary embodiment, again consistent with the teachings above, the apparatus is configured to inhibit and/or suppress signals from the vestibula that travel to the brain by stimulation of tissue of the recipient using the electrical signal(s). Thus, in an embodiment, a principle of operation of the apparatus is at least that of the vestibular prostheses that inhibits or suppresses the balance function by stimulation. Note that this is not mutually exclusive and that the apparatus can also have the functionality and the associated hardware to re-create the feeling of balance and/or to provide sensory substitution. That said, the functionalities can be mutually exclusive. The apparatus can be an apparatus that only inhibits and/or suppresses the signals or could be an apparatus that only re-creates the feeling of balance in the recipient of the apparatus, etc. Thus, in an embodiment, the apparatus is configured to provide another type of stimulation to the recipient different from the stimulation provided by the one or more electrodes to provide balance therapy to the recipient to supplement a decreased and/or eliminated stimulation from the electrodes resulting from a phenomenon detected by the light capture device meeting a set criteria (e.g., there are a certain number of moving objects within a believed field of view of the recipient, the light level is below a threshold or above a threshold, the visual scene does not include sharp images or otherwise is a scene that is not pronounced, the light level has changed rapidly or otherwise within a time period by a certain amount, etc.). The set criteria can be fixed or can be set by the recipient or by a caregiver or healthcare provider. For example, a given light level that is utilized as a criterion can be set based on human factors engineering data or could be set based on subjective data associated with the specific recipient. The light level criteria can be adapted over time based on the changing desires or comfort of the recipient. The criteria can be set utilizing artificial intelligence or machine learning algorithms where the various changes made to the prosthesis by the recipient based on his or her desires with respect to changing environments can be correlated into criteria that can be used by the apparatus as the set criteria.

Note that the concept of a set criteria can be utilized in other scenarios in other embodiments, such as determining whether or not to suppress the vestibular balance function or to inhibit the vestibular balance function, and by how much to suppress or inhibit, etc. Any of the teachings detailed herein associated with varying or otherwise controlling the balance medical device can be applicable to utilizing a criterion such as a set criterion to trigger or control the medical device providing that the art enables such, unless otherwise noted.

In an exemplary embodiment, the apparatus is configured to, based on output from the light capture device, providing notification to the recipient indicative of at least one of a light based environmental feature, such as a light level (qualitative and/or quantitatively) or an action that is recommended to be taken by the recipient of the apparatus. These have been discussed above but briefly, in an embodiment, the notification could be that the ambient light is X lumens or considered dark or considered problematically bright or considered acceptable for implementation of the use of the apparatus as currently set or the opposite. The notification could be that the visual environment has a number of moving objects or could be one that could influence the balance of the recipient (or some other notification that conveys the goal without insulting the intelligence of the recipient but also in a concise manner). And note that the apparatus need not necessarily provide the notification. Instead, the apparatus could initiate the notification. In this regard, the apparatus could provide a signal to the accessory device/assistant device or to the smart phone, etc., indicative of the notification that should be given, and the smart phone or the assistant device could provide the notification. This could have utilitarian value with respect to embodiments where the notification is a text message or an audible message utilizing voice, where the apparatus does not include a speaker but the smart phone does include a speaker. That said, a speaker could be built into the apparatus, such as the behind-the-ear device, and can be configured to output a verbal message.

The notification could be an action that is recommended. In this regard, as briefly noted above, the notification could be a warning to take care. The notification could be more specific, such as to walk slower or more carefully or to take smaller steps or to focus on a fixed object (depending on the sophistication, the apparatus could identify the object-embodiments can include a laser apparatus that could illuminate a “target” and notify the recipient that he or she should focus his or her sight on that limited target). The notification could be to make an adjustment to the apparatus, such as in embodiments where the recipient wants to have more control over the apparatus and the apparatus is not automatically adjusting its performance including activation and deactivation based on the environment. For example, if the apparatus determines that the light level has sufficiently decreased to a level that could be problematic, the apparatus could provide a recommendation to the recipient that the recipient deactivate the stimulation portion of the apparatus until subsequently notified by the apparatus.

In an embodiment, the apparatus is configured so that the control unit at least one of increases a threshold of activation the electrodes, increase a stimulation rate of the electrodes or increases an amplitude of the electrical signal(s) based on output from the light capture device. In an embodiment, the apparatus is configured so that the control unit at least one of decreases a threshold of activation the electrodes, decrease a stimulation rate of the electrodes or decreases an amplitude of the electrical signal(s) based on output from the light capture device.

Embodiments include a human balance medical system, comprising a neurological stimulator subsystem configured to influence neurological signals to a brain of a recipient of the human balance medical system to improve balance of the recipient. The system includes a power source, such as any of those detailed herein. The neurological stimulatory subsystem of the system is powered by the power source, and the human balance medical system is a smart human balance medical system. With regard to the “smart” aspects of the human balance medical system, this can be achieved by the embodiments described herein relating to the control of the prosthetic components based on the ambient environment and/or based on the comfort levels of the recipient or otherwise the physiological state of the recipient. With regard to the latter, it is noted that any embodiment disclosed herein relating to the comfort level of the recipient can correspond to a disclosure of a more generalized physiological state of the recipient. In this regard, for example, the various sensors utilized to obtain data based on latent variables associated with comfort level can be utilized to assess various physiological features of the human, at least in some embodiments. In an embodiment, consistent with the teachings detailed above and just mentioned, the smart human balance medical system is configured to control the neurological stimulator subsystem based on input into the system indicative of an ambient environment of the system. In an embodiment, the smart human balance medical system is configured to automatically adjust an environment of the system to improve efficacy of the neurological stimulator subsystem. This can be implemented according to any of the teachings herein. But briefly, it is noted that in an exemplary embodiment, the human balance medical system can be in signal communication with another system, such as a household system or a workplace system or a car system or some other transportation system, that can be controlled based on a signal from the human balance medical system. Embodiments of the system could be controlled by other means, such as by some other smart system associated with that other system. For example, households can include a system where lights can be adjusted based on the presence or absence of a human, which presence or absence can be detected by a sensor subsystem of that system. In a similar vein, that system could be adapted for use with the smart human balance medical system. The smart human balance medical system could provide control instructions to the other system. That said, in a different embodiment the smart human balance medical system could simply provide data relating to the environment or the like and/or relating to the physiological state of the recipient or the comfort level of the recipient, and the other system can analyze that data and take action accordingly. The utilization of the Internet of things concept can enable these embodiments in at least some instances.

In some embodiments, the smart human balance medical system is configured to automatically adjust a functionality of the neurological stimulator subsystem based on ambient light and/or noise level. The smart human balance medical system could be configured to automatically adjust a functionality based on other variables as these are not mutually exclusive. And consistent with the teachings above, in an embodiment, the smart human balance medical system is configured to automatically halt a functionality of the neurological stimulator subsystem based on an ambient light level and/or ambient noise level, or any of the other “trigger” events detailed herein. With respect to halting the functionality, this can be, for example, with respect to the embodiment of the inhibition and/or suppression of the vestibular balance function, halting of the application of electrical signals or otherwise halting the inhibition and/or suppression of the vestibular balance function by the prostheses.

In an embodiment, the smart human balance medical system is configured to automatically check an ambient light level and automatically, based on the checked ambient light level, selectively:
(i) adjust a functionality of the neurological stimulator subsystem;
(ii) halt a functionality of the neurological stimulator subsystem; or
(iii) provide information and/or a recommendation to the recipient to improve balance of the recipient.

This captures the various features detailed above with respect to at least some of the various permutations herein. Briefly, the action of adjusting a functionality of the neurological stimulator subsystem can include reducing or increasing the inhibition of the vestibular balance function. The action of halting the functionality can include halting electrical stimulation to suppress the vestibular balance function. The providing of information and/or a recommendation to the recipient can include the above-noted information's and recommendations or any other recommendation or information that can have utilitarian value. In an embodiment, the smart human balance medical system is configured to gather latent variable(s) that can impact recipient cognitive load and automatically, based on the gathered latent variable(s), selectively:
(i) adjust a functionality of the neurological stimulator subsystem;
(ii) halt a functionality of the neurological stimulator subsystem; or
(iii) provide information and/or a recommendation to the recipient to improve balance of the recipient.

The latent variables can be those detailed above, such as body temperature or the EEG are EKG values, and ambient light level or the presence of objects that are moving around the balance impaired human, etc. Anything that can be indicative of the cognitive load to the recipient or which could impact the cognitive load of the recipient can be utilized. Speech rate and pronunciation can be used (the idea is that a person who is not speaking clearly or accurately may be cognitively burdened).

Embodiments thus include devices and/or systems configured to receive input indicative of latent variables or the like that are indicative of the recipient becoming, for example, fatigued, the recipient having less cognitive capability than that which was previously the case and/or that the sound and/or light to which the recipient is being exposed requires more effort to comprehend. In an exemplary embodiment, the devices and/or systems can correlate input relating to the latent variables and can train itself to automatically take an action upon the presence of data that indicates that an action should be taken (because an action had been repeated taken in the past when such data was present).

Embodiments can utilize any one or more of the teachings of US patent application publication number 2017/0304620 to Dr. Sean Lineaweaver and John Michael Heasman, published on Oct. 26, 2017, to evaluate/ascertain cognitive load and/or to capture/sense latent variables that can be utilized to evaluate/ascertain cognitive load, or to ascertain a feature of the environment that can be utilized to make one or more of the determinations herein regarding how to affect the vestibular balance function of the human, etc. For example, embodiments can include utilizing any one or more of the features of that patent application related to determining/estimating cognitive load and using that as a basis/trigger for taking any one or more of the actions herein relating to adjusting/using the balance prostheses detailed herein.

As noted above, embodiments can include affirmatively taking control of an environment of a balance impaired person. In this regard, embodiments are not just limited to control of the balance sensory device, but can include, potentially to the exclusion of the control of the balance sensory device, the adjustment of the ambient environment. Briefly, it is noted that by use of the Internet of things, by way of example, a medical device can be utilized to control or otherwise prompt a change in the environment to improve the efficacy of the medical device, here, a balance sensory device. In an exemplary embodiment, this could include increasing or decreasing an ambient level of brightness. Indeed, in some embodiments, it can be that a less bright environment has a more calming effect or otherwise makes the recipient feel more comfortable. Embodiments can include adjusting vestibular stimulation from a prior output/setting. For example, vestibular stimulation output (as measured by current level, pulse length, frequency, or any combination thereof) could be increased by 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 80, 90, 100, 150, 200, 250, 300, 350 or 400% or more or any value or range of values therebetween in 0.1% increments coupled with a decrease in light, by for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, or 70% or more or any value or range of values therebetween in 0.1% increments, and/or by 15, 20, 25, 30, 35, 40, 45, 50, 55, 60, 65, 70, 80, 90, 100, 110, 120, 130, 140, 150, 160, 170, 180, 190, 200, 225, 250, 275 or 300 or more lux. or any value or range of values therebetween in 0.1 lux. increments. The decrease in light could take place within 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 80, 90, 100, 110, 120, 130, 140, 150, 160, 170, 180, 210, 250, 300, 350, 400, 450, 500, 600 or 700 seconds or any value or range of values therebetween in 0.1 second increments.

Note also embodiments can include the reverse, such as reducing stimulation output and increasing the lighting by any of the amounts noted above (but in reverse). Embodiments can include reducing stimulation output and reducing lighting, increasing stimulation output and increasing lighting, etc. such increases or decreases being according to the above (for the purposes of textual economy, any disclosure of a decrease or increase corresponds to a disclosure of the opposite values unless otherwise noted, providing that the art enables such). Any combination of adjustments that can have utilitarian value can be practiced providing that the art enables such.

In an exemplary embodiment, there can be an implementation of a method where the output simulation level is increased by 5% or 10% coupled with a decrease in light from 300 or 250 Lux to 150 or 125 Lux, by way of example. This can have the subjective impact on the balance impaired person of making the balance impaired person feel more comfortable than that which was the case prior to these arrangements. Conversely, it could be that if the light level is reduced too much, such as by way of example, below 100 Lux or below 50 or 70 or 60 Lux, the balance impaired person can start to feel uncomfortable. And it could be such that even if stimulation is increased, there is little to no efficacy. Instead, other types of stimulation could be applied which could be more efficacious. Such other stimulation could be, for example, the acoustic stimulation detailed above. In these embodiments can have utilitarian value with respect to power management of the medical device. In this regard, these devices can be power intensive because of the electrical stimulation to the tissue, relative to other types of medical devices, such as a hearing aid (generally speaking). There can be utilitarian value with respect to limiting power output or otherwise not abiding electrical stimulation when the stimulation will have little to no benefit. In this regard, increasing longevity of a medical device with respect to time periods between recharging or changing batteries can have utilitarian value. Corollary to this is that a full-blown increase in stimulation, such as operating the balance sensory device at maximum output, could quickly discharge the batteries, or more accurately, more quickly discharge the batteries relative to that which would be the case at a lower output level. In this output level is achieving an efficacious results, it could be warranted. What if this output level is not achieving utilitarian results, it is unnecessary and otherwise wasteful and impacts the overall efficacy of the treatment in that the balance sensory device will become discharged faster than that which would otherwise be the case, thus reducing its overall utilitarian value in the short run at least.

Accordingly, embodiments include utilizing an ambient environment as a latent variable to evaluate the efficacy of stimulation, and controlling stimulation or otherwise controlling the balance sensory device based on how efficacious use thereof during a given scenario would be. This concept can be somewhat counterintuitive, because the goal of a medical device is typically to perform, and to perform in all instances. But the present inventors have determined that not only is there utilitarian value in limiting or halting the effects of a balance prosthesis on a human with respect to preventing a deleterious effect, such as suppressing the vestibular balance function in a lowlight environment, but that there is also utilitarian value in controlling a balance prosthesis based on the efficacy of that device in the first instance. Put another way, whereas the former utilizes the end goal of the outcome for the human as the control (e.g., what action can be taken to increase the level of balance experienced by the human), the latter utilizes the fact that the outcome for the human is essentially fixed, and adjusts the balance sensory device accordingly for the greater goal of maintaining compliance for example by extending the battery life beyond that which would otherwise be the case.

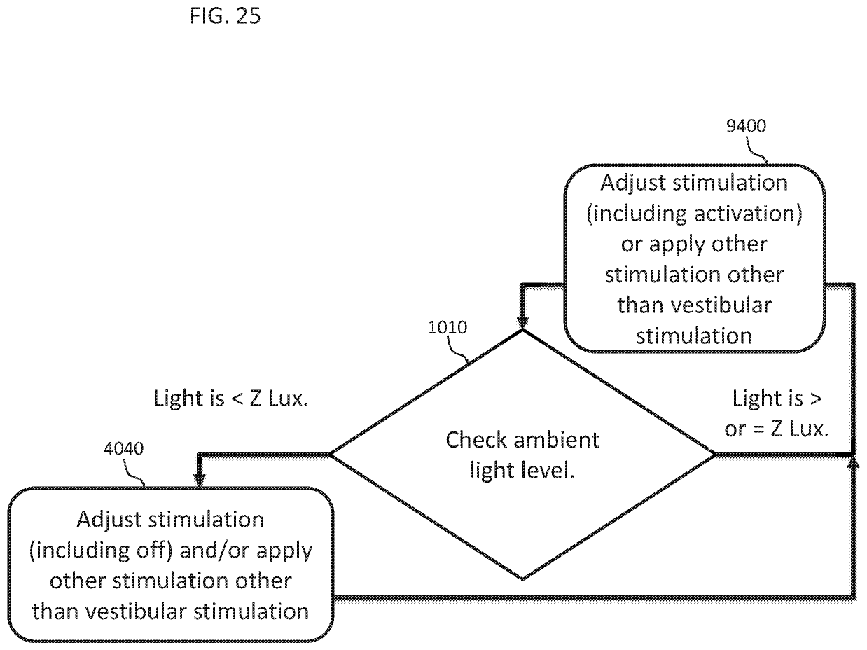

FIG. 25 shows another exemplary flowchart for an exemplary method, where method action 1010 is as above. Here, if the light level meets or exceeds a certain value, the method proceeds to method action 9400, which corresponds to method action 4020 above. If the light level is below a certain value, the method proceeds to method action 4040, which is as above. It is briefly noted that while the equal qualifier is presented on the right side of the flow charts, in an embodiment, that qualifier could be on the left side (the right-sided left side is utilized as shorthand).

The embodiment of FIG. 25 can take into account the fact that at certain light levels, such as, for example, light levels below 50 or 40 or 30 Lux or so, statistics indicate that stimulation from the balance sensory prosthesis provides little if any increase in balance performance. This as compared to, for example, no stimulation at all. That is, no stimulation at all could provide the same results as stimulation at the maximum output possible. And in this regard, while method action 9400 includes various scenarios and method action 4040 include various scenarios, in an exemplary embodiment of the flowchart of 25, method action 9400 could simply be to turn on or otherwise activate the implant, and commence stimulation from where previously there was no stimulation, and method action 4040 can entail simply turning off the stimulator or otherwise halting stimulation whereas there was stimulation prior thereto.

Still, embodiments can include a sliding scale where an increase in output can be deemed to have little to no improvement, and thus the adjustment in method action 4040 would be to reduce the stimulation to a level that is deemed to have utilitarian value for that light level.

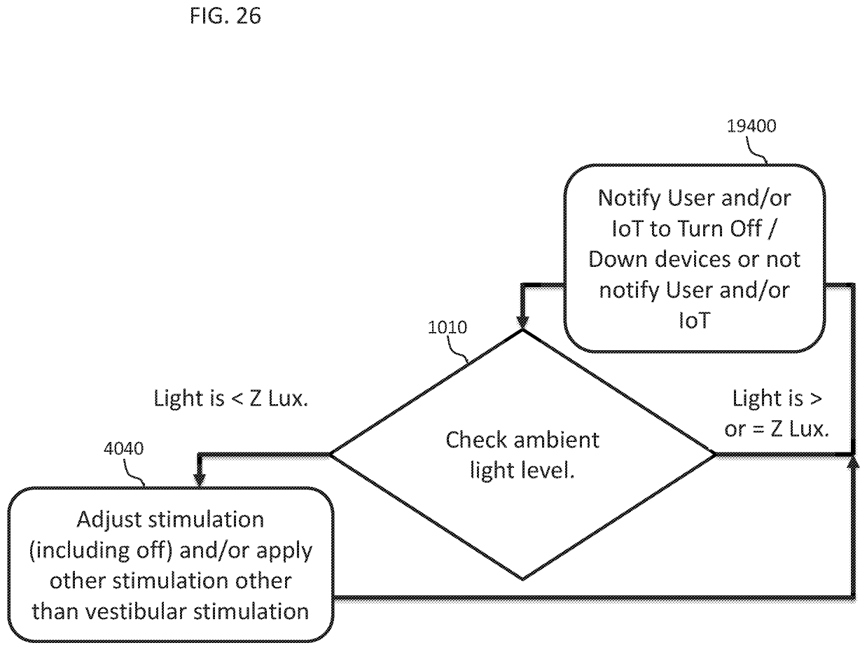

FIG. 26 presents another exemplary flowchart which parallels the flowchart of FIG. 25, except that there is instead method action 19400, which includes the action of notifying the user and/or the Internet of things to turn off or down devices or to not notify the user and/or the Internet of things.

It is briefly noted that while the flowcharts of the methods of FIGS. 25 and 26 do not indicate the prior state of the balance sensory device, the balance sensory device could be in a stimulating state for a non-stimulating state depending on the circumstances. And it is also briefly noted that while many of the above-noted methods indicate that there is a “start stimulation” in method action 940, that can also include the scenario where the vestibular stimulation has been ongoing. That is, any disclosure of starting stimulation corresponds to an alternate disclosure of being in a stimulated state, and vice versa, providing that the art enables such, unless otherwise noted, all for the purposes of textual economy.

The embodiment of FIG. 26 can have utilitarian value with respect to scenarios where acoustic noise can utilitarian value as well. By way of example, acoustic noise can work like a linear reaction between the output level in noisy environments and balance performance. There can be scenarios where improvements to balance are gained in linear proportion to reductions in environmental noise. In the reverse can also be true. And note that in some embodiments, in an exemplary embodiment, the reaction can be non-linearly correlated. Still, the point is that in some exemplary embodiments, turning down a radio or a television or executing some form of noise cancellation can improve the efficacy of the balance sensory prostheses. Still further, in some embodiments, this could be the case irrespective of whether or not an adjustment is made to the balance sensory prosthesis or even whether or not the balance sensory prosthesis is providing stimulation to the recipient. By way of example only and not by way of limitation, there could be a scenario where the light level is sufficiently low that stimulation would be non-utilitarian, and thus the stimulation is turned off. However, the device could still notify the user and/or notify the Internet of things or whatever devices are in a system that are controlled by or controlled based on input from the medical device, to take some form of action to improve balance. Thus, even though the light level is such that it may warrant turning off stimulation, the medical device can still improve balance by performing these other ancillary features. This is consistent with the concept of providing alternate stimulation. But this is also consistent with the concept of providing noise cancellation and/or reducing the sound level in an environment.

And it is briefly noted that as used herein, the action of applying other stimulation other than vestibular stimulation could include adjusting that other stimulation, such as adjusting the noise level.

With respect to noise cancellation, embodiments can include the utilization of hearing aid technology to cancel the noise. In an exemplary embodiment, in the ear devices could be utilized to transpose sound to the recipient, where the recipient does not have a hearing problem or otherwise does not need per se a hearing aid. The hearing aid could actually operate potentially in reverse, so as to dampen/deamplify the sound, at least for certain frequencies. The end the ear device can be utilitarian because it can at least partially occlude the ear canal. Embodiments can alternatively and/or in addition to this include a speaker or the like that outputs a sound that has the effect of canceling noise, which speaker could be located on a behind-the-ear device for example.

Still, embodiments include instructing the user and/or the Internet of things or some other device to take action to change the environment to improve balance or at least not further detract from balance irrespective of the presence or absence of vestibular stimulation.

Embodiments also include treating oscillopsia. In this regard, one or more or all of the teachings detailed herein can be applicable to the treatment of a person who experiences oscillopsia. Any disclosure herein of a balanced impaired person or person in general corresponds to an alternate disclosure of a person who is afflicted with or otherwise experiences oscillopsia (and the two are not mutually exclusive at least in all instances). In an embodiment, the combination of light level control and stimulation control are utilized to treat oscillopsia. In an embodiment, this treatment can be directed towards children or young adults or infants. In an exemplary embodiment, this treatment is directed toward people who are less than, 21, 20, 19, 18, 17, 16, 15, 14, 13, 12, 11, 10, 9, 8, 7, 6, 5, or 4 years of age or any value or range of values therebetween in one month increments. In this regard, there can be light levels that are more comfortable to the person than others, and there can be a correlation between balance and other vestibular related features, such as eye-movement and eye tracking, and ambient light level. Embodiments include ascertaining the utilitarian combinations of ambient light level and stimulation for a particular person, whether strictly subjective or based on statistical values for a greater population or combination thereof, to treat oscillopsia. Embodiments can be directed towards improving a comfort level or otherwise achieving a comfort level, where a comfort level can change or otherwise impact a real-time cognitive level of a human. Without being bound by theory, in at least some exemplary embodiments, the efficacy of vestibular stimulation utilizing the medical devices herein and variations thereof and other medical devices, can be enhanced if the person has less cognitive load relative to that which would otherwise be the case. Put another way, if a person is not concentrating on other things, or otherwise afflicted with a high cognitive load, the outcomes of the vestibular stimulation can be superior. Accordingly, embodiments include reducing the cognitive load of a recipient in combination with the application of stimulation therapy. Such reductions in cognitive load can be executed utilizing at least in part the teachings of the above-noted patent application to Dr. Sean Lineaweaver, such as by way of example only and not by way of limitation, utilizing the various teachings therein to ascertain or otherwise estimate cognitive load, and/or utilize the various teachings therein to reduce cognitive load.

Briefly, embodiments include utilizing a vestibular stimulation device to dampen the vestibular balance function. Embodiments include halting the damping or limiting the damping relative to that which would otherwise be the case depending on the environment. In an embodiment, the balance sensory device is configured to dampen the vestibular function by at least and/or equal to 5, 20, 25, 20, 25, 30, 35, 40, 45, 50, 55, 60, 65, 70, 75, 80, 85, 90, 95 or 100% or any value or range of values therebetween in 1% increments. Thus, embodiments that adjust the balance sensory medical device can change to any one or more of the just-noted percentages from any one or more of the just noted percentages based on the environment. This accounts for increase and decrease. Also, an embodiment can decrease an output level (one or more of the above-noted features of the electrical stimulation) by 5, 20, 25, 20, 25, 30, 35, 40, 45, 50, 55, 60, 65, 70, 75, 80, 85, 90, 95 or 100% or any value or range of values therebetween in 1% increments depending on the changed environment. The reverse can be true, although the upper limit is not bounded by 100%, as the increase could be greater, such as to 200, 300, 400 or 500% or any value or range of values therebetween in 1% increments. Embodiments can also be classified by the power consumed by the stimulation. This can be a proxy for output. Thus, the above percentages can be applicable to the power required to stimulate (as differentiated from the power required to maintain the implant, for example, above the reset voltage and/or at the standby voltage, or to provide back telemetry, etc.). This is the power that is required to stimulate/the power required to move current out of the electrodes (and into the electrodes for an alternating system). Accordingly, in an embodiment, the stimulation power can be reduced and/or increased by the above-noted percentages.

Any reference to a vestibular stimulation device refers to a single device applying stimulation to one ear, a single device applying stimulation to both ears and a plurality of devices applying stimulation to both ears, and vis-a-versa, unless otherwise noted.

In an exemplary embodiment, there are devices, systems and/or methods that are utilized to provide an indication that the sensory management and/or sensory stimulation systems detailed herein and variations thereof and/or other types of systems that involve medical devices which can be other types of sensory management and/or sensory stimulation systems and/or could be other types of systems that are medical devices that are not sensory stimulation and/or a sensory management systems, is/are activated and/or is/are not activated. In an exemplary embodiment, this indication is provided automatically by the system, which can be provided by the prostheses and/or a body worn device that constitutes the medical device and/or another component that works in conjunction with the prostheses, such as for example, a smart phone or a smart watch, or a dedicated remote control for the prosthesis (or a nondedicated remote control, which can be the handheld device of system 210 by way of example only-more on this below). In an embodiment, there are devices, systems and/or methods that are utilized to provide an indication that the aforementioned systems are being implemented in an efficacious manner and/or that provide an indication of a level of efficaciousness of the implementation, as an alternative and/or in addition to the indication that the system is activated or otherwise has been activated.

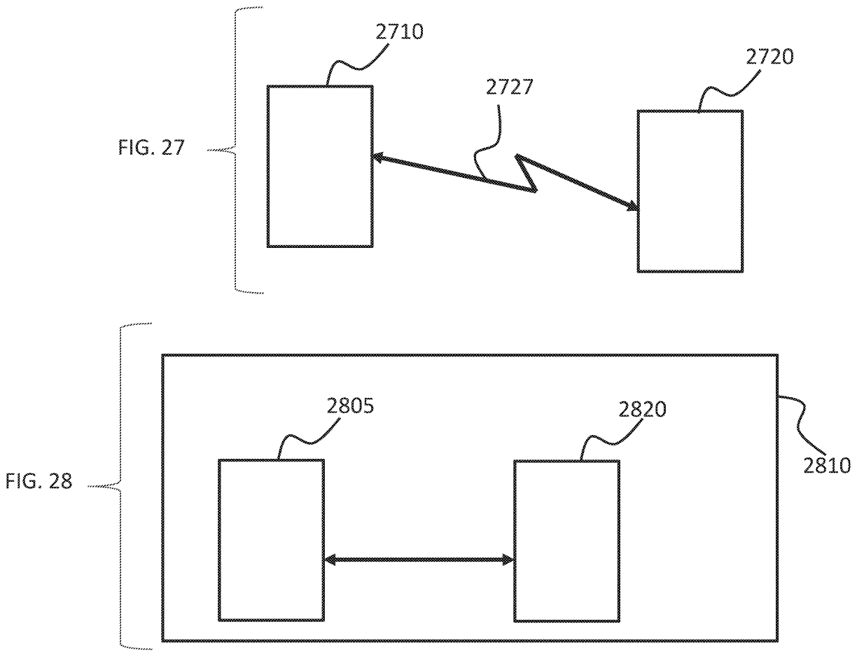

In an embodiment, there is a system, such as that shown in FIG. 27, which includes a first subsystem 2710 configured to neurologically affect a human when activated. The system includes a second subsystem 2720 that is configured to provide an indication that the system is activated and/or not activated. As will be described briefly below, the system can include a third subsystem and/or one of the first subsystem and/or second subsystem can be configured to make a determination whether or not the system has been activated, which determination can be made automatically. The two subsystems communicate with each other via radio frequency link 2727 as shown which can be in some other embodiments a wired link. In an exemplary embodiment, the system of FIG. 27 corresponds to system 210, where subsystem 2710 can be the prostheses 100 and subsystem 2720 can be the smart phone 2410 or can be some other smart device or a non-spart device, such as a dedicated remote control. In an exemplary embodiment, the prostheses 100 as utilized in the arrangement of FIG. 27 can be a cochlear implant and/or can be a vestibular stimulation system as detailed above and/or a tinnitus management device as will be described below. The vestibular stimulation system can include, in some embodiments, the aforementioned environment sensing arrangement, which can include the light sensor(s) detailed above. That said, in some exemplary embodiments, the vestibular stimulation system does not include the light sensor, for example, or the environmental sensing arrangement, detailed above at least not as utilized according to the teachings above.

FIG. 28 presents another exemplary system where the two subsystems are contained in a single prosthetic device 2810, which can be a vestibular stimulation system, balance sensory medical device or a tinnitus management/mitigation device, for example. Subsystem 2805 can be the subsystem configured to neurologically affect a human when activated, and subsystem 2820 can correspond to subsystem 2720 detailed above. As seen, the two subsystems can communicate with each other, here, in a wired/printed circuit manner and/or by light communication, although in other embodiments, the communication can be wireless. In the exemplary embodiment of the embodiment of FIG. 28, both subsystems are contained in a single housing, which can be an implantable housing, and thus both subsystems are implanted (and this can include the subsystem that makes the above-noted determination of activation in some embodiments). In an alternate embodiment, the subsystems are located in separate components of the prostheses, such as for example the implantable component with respect to the first subsystem and the external component with respect to the second subsystem. In some embodiments, the first and second subsystems are located in the external component (and there might be no implantable component depending on the system, or vis-a-versa). Note also that the individual various subsystems can be divided up between the various components. By way of example only and not by way of limitation, a portion of the second subsystem can be located in the implantable component and a portion of the second subsystem can be located in the external component (as well as the first subsystem).

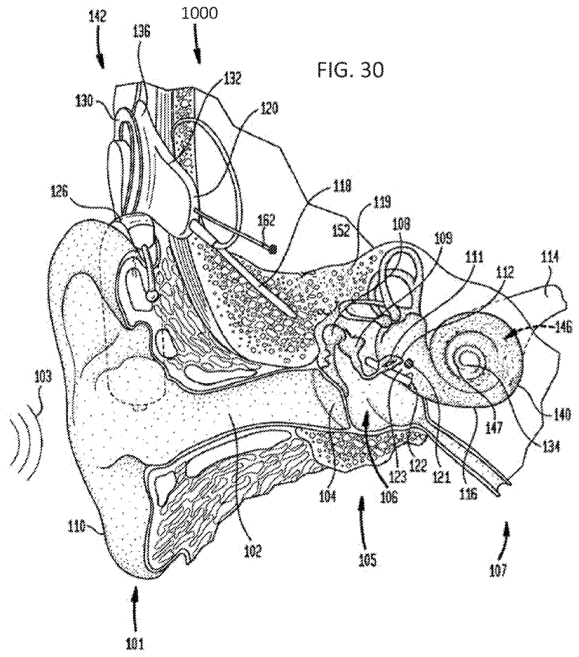

Briefly, FIG. 30 is a perspective view of a tinnitus treatment implant, referred to as implant 1000, implanted in a recipient. As shown, implant 1000 comprises one or more components which are temporarily or permanently implanted in the recipient. Implant 1000 is shown in FIG. 1000 with an external device 142 which can correspond in principle to that detailed above, but modified/different for tinnitus treatment, the details of which will not be expanded upon for purpose of Briefly, the implant 1000 can be loosely based on a cochlear implant (partially implantable or totally implantable). The arranging of implant 1000 can receive power and/or data in a manner similar to and/or the same as or otherwise as modified to implement tinnitus treating and convert such to an electrical stimulation signal that is ultimately delivered to tissue. As detailed herein, some arrangements may or may not have an implanted processor. Any arrangement that can be used to provide electrical stimulation to an electrode to stimulate tissue to treat tinnitus (or to treat epilepsy, for that matter-more on this below) can be used in some embodiments.

In the illustrative arrangement of FIG. 30, external device 142 (again, based on that device above, but for a tinnitus treatment device) may comprise a power source (not shown) disposed in a Behind-The-Ear (BTE) unit 126. Implant 1000 comprises an internal energy transfer assembly 132 which may be positioned in a recess of the temporal bone adjacent auricle 110 of the recipient. As detailed below, internal energy transfer assembly 132 is a component of the transcutaneous energy transfer link and receives power and/or data from external device 142.

Implant 1000 further comprises a main implantable component 120 and an elongate electrode assembly 118.

The above said, in an exemplary embodiment, the implant 1000 is a totally implantable apparatus that includes a power source (e.g., battery), and is configured to operate in a manner akin to a totally implantable hearing prosthesis, as modified for tinnitus treatment.

Elongate electrode assembly 118 has a proximal end connected to main implantable component 120, and a distal end that includes an electrode that is located abutting the cochlea 140. In the embodiment shown in FIG. 30, the electrode (not shown) is located in drilled partial hole 122. Electrode assembly 118 extends from main implantable component 120 to cochlea 140 through mastoid bone 119, to the otic capsule.

As noted, implant 1000 can comprise a totally implantable prosthesis that is capable of operating, at least for a period of time, without the need for external device 142. Therefore, implant 1000 further comprises a rechargeable power source (not shown) that stores power received from external device 142. The power source may comprise, for example, a rechargeable battery. Alternatively, a long term non-rechargeable power source that is implanted and remains implanted may be used. During operation of implant 1000, the power stored by the power source can be distributed to the various other implanted components as needed. The power source may be located in main implantable component 120, or disposed in a separate implanted location.

As seen, there is a return electrode/reference electrode at the end of lead 162. This provides a return/reference for the electrode at the end of the electrode assembly 118.

Embodiments include utilizing implant 100 to treat tinnitus, such as via the application of electrical current to the ear system of a person suffering from tinnitus. By way of example only and not by way of limitation, in an exemplary embodiment, one or more electrodes are placed against or otherwise in electrical communication with the tissue (e.g., bone) of the otic capsule/bony labyrinth, etc. In an exemplary embodiment, by way of example, one or more electrodes are placed against or otherwise in electrical communication with the round window of the cochlea. The concept here is that the electrical current supplied by the electrode or otherwise conducted from the electrodes to the tissue, will stimulate the inner ear nerves or otherwise the auditory nervous system, in a manner that can be utilized to treat and/or otherwise mitigate the effects of tinnitus.

Embodiments include utilizing the teachings herein to indicate the activation/functionality/efficacy of the tinnitus treatment/masking implant 1000.

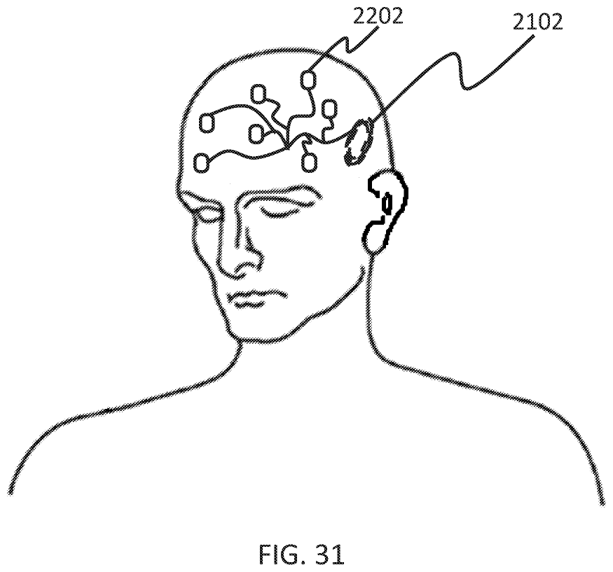

Another exemplary arrangement that can use the technologies detailed here is an epilepsy monitoring and/or treatment device. FIG. 31 provides an exemplary embodiment of an EEG system (that can be an epilepsy monitoring system) that is implanted in the recipient, where read/sense electrodes 2202 are arrayed inside a recipient's head and in signal communication with a coil 2102 via electrical leads. In this embodiment, the implanted device has no recording/storage capabilities, and requires an external device to receive a signal from the implanted inductance coil 2102 so as to retrieve in real time the signal therefrom. Not shown is an implantable component that converts the electricity sensed by the sensor/read electrodes into a detailed herein, again unless otherwise noted provided that the art enables such.

Consistent with the teachings detailed above, in an exemplary embodiment, the system is a human balance medical system, which can correspond to the exemplary embodiments detailed above, in whole or in part, but can also be variations thereof or otherwise different embodiments of a human balance medical system. Embodiments can include devices and/or systems configured to provide and/or determine and/or methods of providing the indication and/or determine that the system is activated/not activated and/or the efficacious operation thereof and/or the functional state thereof. In an embodiment, the system is configured to provide this indication to the signal that is transmitted by the inductance coil. In an exemplary embodiment, the sensor arrangement seen in FIG. 31 is an implanted EEG sensor arrangement.

Embodiments include utilizing the teachings herein to indicate the activation/functionality/efficacy of the epilepsy treatment system of FIG. 31.

Embodiments can also be used with a sleep apnea treatment system.

Any disclosure herein of the utilization of one or more of the teachings described herein with a balance sensory system or a tinnitus treatment system and/or an epilepsy treatment system and/or any of the medical devices detailed herein corresponds to, in the interest of textual economy, a comparable disclosure to utilizing those teachings with any one of the other systems, unless otherwise noted, providing that the art enables such. To be clear, embodiments include devices, systems and/or methods where one or more of the teachings detailed herein are utilized with one or more of the medical devices user of the system, such as the recipient of the prosthetic device that is part of the system. In an exemplary embodiment, the system is configured to provide this indication to a third-party relative to the recipient/patient/user of the system (person directly affected by the system), such as a caregiver or a guardian of the recipient of the prosthetic device by way of example. A third party monitoring company could be provided with the indication. By way of example only and not by way of limitation, a third party could monitor the activation rates and/or the efficacy and/or the functionality, etc., based on the indications that are provided thereto. In an exemplary embodiment, this can have utilitarian value with respect to having a professional organization or otherwise a company that is skilled in the subject matter associated with the system assess the aforementioned phenomenon to determine if there is a “problem” with the system and/or recipient/user of the system to determine whether or not an intervention can be utilitarian. By way of example only and not by way of limitation, the human balance medical system could result in the recipient of the system feeling nauseous. This could then result in the recipient of the system not utilizing the system as much as he or she could be or otherwise should be and thus creating a potentially dangerous situation in the person's life. This data could be utilized by the professional third-party to determine or otherwise evaluate the settings and adjust the system remotely by way of example.

Still, embodiments can be focused on providing the indication to the recipient and/or the immediate caregivers of the person that is affected by the system. The indication can be for example and not by way of limitation, a visual indication, for instance, such as activation and/or deactivation of a light emitting diode (LED), or a change in color thereof, or the presentation of a given sequence of LED activation and/or a change in a sequence of LED activation, or a change in flashing thereof (from non-flashing), or flashing in a first pattern vs. a second pattern, etc. The visual indication can be on the external component of the prostheses (such as the BTE device) that is part of the system or a component remote from the prostheses (in embodiments where the system utilizes a prostheses), such as a handheld smart phone or smart device. The visual indication can be on a remote control unit of the system, such as a hand-held remote control unit for any of the prostheses detailed herein. The visual indication could be a message (smartphone text message or email) or a status indicator on any of these devices. By way of example only and not by way of limitation, the message or the status indicator could be displayed on the screen 2421 of the handheld component 2401 where the system corresponds to the system 210 or a variation thereof detailed above (e.g., the second subsystem could be part of the handheld component 2401).

The indication could be an audible indication in the form of a beep or a tone that can be triggered by an external trigger such as a smart device command. The indication can be a computer generated or pre-recorded voice statement from a speaker of the prosthesis or device or smartphone or remote control.

The indication could be electrical stimulation of tissue of the recipient/user, such as stimulation of a nerve (for instance by way of example only, the auditory nerve for a vestibular implant, and thus the stimulation of the nerve for indication purposes is different from the stimulation of the nerve(s) for vestibular stimulation/balance improvement/maintenance). Indeed, in an exemplary embodiment, stimulation could be a stimulation that parallels or otherwise is analogous to, at least in general terms, that which results from a cochlear implant stimulation. In some embodiments, the implant has electrodes that are also located in or at least at least proximate the inner ear (and in some embodiments in the cochlea) and can provide at least a rudimentary form of stimulation to the auditory system, which stimulation can be perceived as a hearing percept in general, and could be a verbal hearing percept in particular. That said, in an exemplary embodiment, the stimulation can be in the form of a series of pulses akin to a Morris code signal or could be a single tone or a varying tone, anything that is hearing percept based that can convey the indication to the recipient of the system. Embodiments can also be configured to provide a “hi stimulation” relative to that which is normally provided by the system, which hi stimulation is sufficient to stimulate the auditory nerve utilizing electrodes that are located outside the cochlea and/or away from the cochlea. In this exemplary embodiment, with the system includes, for example, a vestibular stimulation device, and the electrodes thereof are located away from the cochlea, the current to those electrodes could be increased and/or the voltage could be increased by a relatively large amount over that which is utilized to stimulate the vestibular system for example, and this amount can be sufficient for the current to stimulate the auditory nerves in a manner that results in a hearing percept of some sort sufficient to implement the teachings herein. The frequency and/or pulse and/or other electrical characteristics that are utilized in this exemplary embodiment could be such that the vestibular system is not stimulated in a manner that affects the balance of the human but will be perceived as sound by the cochlea.

In an embodiment, a haptic indication/feedback can be utilized to convey the indication. Embodiments can include a system that includes a vibration device, such as the vibrator of a portable phone, or a vibrator of a bone conduction device. The vibration output can be triggered by an external trigger such as a smartphone command. In an exemplary embodiment, the vibration can be presented in a manner analogous to the above-noted LED presentation, where a sequence can be provided that conveys a certain indication by way of example. A pattern or intensity or a frequency or any one or more combinations of these can be utilized to convey the indication. This is the case for the light indication or the sound indication of the tactile indication, etc.

Any device, system and/or method of providing/conveying the indication that can have utilitarian value can be used providing that the art enables such. To be clear, embodiments include a device and/or system configured to provide indication delivery and/or a method of delivering the indication by any one or more or all of a smart device (smart phone for example, or tablet, or smart watch), or remote control of a medical device), a dumb device (e.g., a digital watch or a neck worn pendant for example that has a receiver that receives a signal from another component of the system indicative of the status of the system, such as that the system is activated and/or not activated, which can also include an arrangement that provides an indication upon lack of receipt of the signal from the another component of the system), a BTE or an OTE or an ITE device, a car communication system, infrastructure, the Internet of things, etc. Embodiments include a device and/or system configured to provide indication delivery and/or a method of delivering the indication by an audio arrangement, a visual arrangement, a tactile arrangement, an electrical arrangement, which can include shocking the recipient or otherwise providing a “zap” or at least a “tingling sensation” to tissue of the recipient for example.

With respect to the indication(s), in an exemplary embodiment, the second subsystem is configured to provide an indication that the system is on and/or that the system is off. In an exemplary embodiment, the system is on when an “on” button (which can be an on/off button) thereof is depressed as needed to turn the device on, and the resulting state of the system is on as one would expect and want (when the system is operating correctly—again, this is based on the operation of the button). This could be a button on the external component of the vestibular implant by way of example, which could be on the BTE device or on the OTE device thereof. This could also be by way of example when an on signal is provided from a portable handheld device, such as a smart phone or remote control, that is in signal communication with the vestibular implant. This does not necessarily require a button in the traditional sense in that a touchscreen can be utilized. Of course, the origins could be verbal input as well. The system is off when for example, the “off” button thereof is depressed to turn the system off, or at least to turn off the ultimate stimulation device, and the resulting state of the system is off as one would expect and want (when functioning properly), again with the aforementioned qualifications albeit with respect to off instead of on.

This is not to say that when the system is on, the system is functioning in a utilitarian manner and/or in an efficacious manner. By way of analogy, a car could be on but if the transmission is broken, the car will not operate as intended. The air conditioner may work but the car will not drive forward or backwards.

Accordingly, in view of the above, the second subsystem can be configured to provide the indication to a recipient of the system via an internally applied stimulus by the system. In an exemplary embodiment, according to the teachings above, the second subsystem can be configured to provide the indication to a recipient of the system via an externally applied stimulus by the system. In an embodiment, the second subsystem can be configured to provide both an internally applied stimulus and an externally applied stimulus. Of course, there could be a third subsystem that can provide the stimulus external to the recipient while the second subsystem provide the stimulus internal to the recipient.

In an embodiment, the second subsystem is configured to directly provide the indication to a recipient of the system from a body worn portion of the system. In an embodiment, this could be the BTE device of the vestibular stimulation system or of the cochlear implant or of the tinnitus management system by way of example. In an embodiment, this could be from a smart watch. With respect to the BTE device, that would also be a second subsystem that is configured to directly provide the indication to a recipient of the system from a portion of the device that is body worn, whereas the smart watch, while part of the system, is not part of the vestibular stimulation implant/device. In an embodiment, the feature of the direct provision of the indication of the recipient could be accomplished utilizing the audible of the tactile limitations detailed above. In an exemplary embodiment, this could also be visual, but such could require for example the recipient to remove the BTE device from his or her ear so that the device can be moved to see the LEDs for example. Of course, embodiments can include using a mirror, or having another person tell the recipient that there is a light that is blinking for example on his/her BTE device. But these would be indirect providing of the indication to the recipient. These would be direct providing of the formation to a third party for example such as a caregiver, who can see the BTE device while the BTE devices on the ear of the recipient. Accordingly, embodiments include a second subsystem configured to provide the indication directly to a third-party relative to the recipient.

Briefly, it is noted that embodiments are configured so that the second subsystem provides an indication that the system is activated. This can be a periodic indication by way of example to the recipient. For example, every five minutes or 10 minutes, the indication can be provided. The idea is to give an indication in an efficacious manner without overwhelming or otherwise even annoying the recipient with “constant” reminders. More on this below. Conversely, embodiments include a system where the second subsystem provides an indication that the system is not activated. In some instances, this could be more utilitarian from a standpoint of recipients of annoyance or tolerance of the implementation of the teachings herein. The system would notify or otherwise provide the indication when the system is not activated. The presumption would be that if this indication is not provided, the system is activated. Of course, embodiments can provide both. In an embodiment, the recipient can select a mode where the indication is that the system is activated, and then select another mode where instead, the indication is that the system is not activated (and the opposite does not occur/is foreclosed). In an embodiment, there can be a third mode where the second subsystem provides both an indication that the system is activated and an indication that the system is not activated depending on the scenario. Moreover, in an embodiment, the system can be configured so that the recipient or a third-party can set the trigger time or the manner in which the indication is triggered. For example, if the recipient chooses the mode where the indication is that the system is activated, the recipient can set the timing of such indication, such as, for example, every five minutes or every 10 minutes or every half hour for example, the indication is provided. This can be a preference for the recipient depending on how often the recipient is comfortable being notified.

Indeed, embodiments include adjusting the trigger depending on a lifestyle action of the recipient. For example, if the recipient is driving, the indication can be provided more frequently than if the recipient is lying in bed reading. The idea is that it would be more critical for the recipient to know if the system was activated or not activated etc. when the recipient is driving as opposed to when the recipient is lying down in the bed. The recipient might very well want to know every minute (or every second, or continuously) while he or she is driving that the system is activated. Indeed, embodiments could be implemented here the system is constantly providing an indication, such as a constant tone for example or some other constant type of stimulation. For example, a recipient may be fine with a low-frequency or high-frequency constant tone or a medium frequency constant tone, which is constantly on or otherwise constantly being “played” while the recipient is doing a given activity, such as driving for example. In this exemplary scenario, the recipient is engaging in an activity that he or she deems to be that which warrants constant notification that the balance device is activated. Of course, this could be a more periodic arrangement where the indication is provided once every 10 second or 20 seconds or 30 seconds or one minute or two minutes etc. Note also that the indication need not be as pedestrian as a tone or some other noise per se. The indication could be that there is music playing or the output of a radio station which is streamed to the medical device is constantly being played (e.g. the recipient can hear the results thereof) while the device is activated, and when the device becomes deactivated, what is being played is stopped, which stopping can be the indication providing that the recipient understands such and otherwise is notified that is the way this will operate. This can be instead a car radio for example, where the system interfaces with the car entertainment system or communication system is outputting the product of the radio station to which the recipient has tune the radio thereto.

Accordingly, the indication can be provided through infrastructure.

Again with respect to the antithesis of constant indication, in an alternate embodiment, indication only occurs when the system is no longer activated. This too can be accomplished via infrastructure such as a car entertainment system etc.

Still, returning back to the concept of the user selected mode or sub mode of indication, the user or a caregiver or the like can select how frequently the indication will be provided for under what circumstances the indication will be provided. While the scenarios above focused on the concept of the system being activated and the indication being delivered periodically, in an alternate embodiment, there could be a lag between the time that the system becomes deactivated or otherwise ceases to be activated and the indication. For example, the user could set the system so that the indication is provided only after 15 seconds or 30 seconds of non-activation by way of example or after a number (predetermined number) of determinations that the system is inactive. That said, in an embodiment, there could be a built in lag time of the device itself where the device double checks or triple checks a determination that it has been deactivated or otherwise that it. The activation or inactivation exists for more than a certain period of time. This would be, in some embodiments, far shorter than the aforementioned 15 or 30 seconds. This could be a time sufficient to ensure that there are not random signals that indicate a “false positive” or “false negative” by way of example. That is, the software is configured to reduce the likelihood that the indication is provided incorrectly or unnecessarily.

Embodiments include utilizing the subsystem configured to provide an indication that the system is activated and/or not activated in conjunction with the above-noted teachings where the system includes a subsystem configured to obtain data based on an ambient environment of the system, where in such an embodiment the system includes a third subsystem that includes a light sensor. As noted above, at least some of the systems are configured to control the first subsystem, at least in part, based on the obtained data from the subsystem that is configured to obtain data based on the ambient environment. The aspects associated therewith can be combined or otherwise utilized with the teachings herein pertaining to the indication, and/or vice versa. By way of example only and not by way of limitation, there are scenarios where the system could halt or otherwise inhibit the amount of stimulation provided by the medical device based on the ambient environment. By way of example only and not by way of limitation, as noted above, the system could automatically limit or stop stimulation of the vestibula if the system determines that there is not a sufficient level of ambient light, or otherwise that it is dark where the recipient is located. In some embodiments, this could result in the deactivation of the system, at least where the system is not providing any stimulation at all by way of example. Accordingly, there can be utilitarian value with respect to providing indication to the recipient or a caregiver or some party that the system is no longer providing stimulation to the human or otherwise neurologically affect in the human.

Indeed, in an exemplary embodiment, the system is configured to limit a level of effect of the first subsystem on the human based on that obtain data pertaining to the ambient environment. Thus, the system that is configured to provide an indication to the human can be configured to do so automatically when the level of effect of the first subsystem is limited, which can correspond to an indication that the system is not activated if the limitations on the effect of the first subsystem corresponds to such. Corollary to this is that an indication can be provided when the system is providing the stimulation based on the ambient environment. Accordingly, for example, if the system determines that there is sufficient light in the ambient environment to implement stimulation, the system could provide an indication that the system has been activated. And note that while embodiments have focused on activation and non-activation, embodiments can also focus on reactivation and deactivation. Any disclosure herein associated with activation corresponds to an alternate disclosure of reactivation and any disclosure herein associated with non-activation corresponds to an alternate disclosure of deactivation unless otherwise noted providing that the art enables such.

In view of the above, it is to be understood that any disclosure herein of managing the medical device systems based on the ambient environment and/or management of the system based on any of the phenomenon detailed herein corresponds to a disclosure of utilizing the teachings associated with providing the indication there with and vice versa, providing that the art enables such, unless otherwise noted. Thus, any disclosure of any embodiment associated with controlling the first subsystem or otherwise the subsystem configured to neurologically affect a human when activated corresponds to a disclosure of a device and/or system for and/or a method of providing indication based on the results or otherwise based on features associated with that management. By way of example only and not by way of limitation, the system could include a fourth subsystem that analyzes the state of the system to determine whether or not the first subsystem is being controlled based on the data obtained by the second subsystem, and this fourth subsystem could control the second subsystem to provide the indication(s) according to the teachings detailed herein.

In an embodiment, the indication can be controlled or otherwise linked to other phenomenon as well. By way of example only and not by way of limitation, the system can be configured to provide the indication when the recipient begins movement even though the trigger for the indication would have otherwise occurred earlier. For example, in the scenario where the low light level of the ambient environment results in the deactivation of the stimulator of the balance system, the system can be configured to delay the action of providing the indication until a point in time where the recipient begins to move. The idea being is that the recipient does not necessarily need to know that the system is in a deactivated state until he or she begins to move. Alternatively and/or in addition to this, the system could evaluate whether or not the recipient is sitting or standing or laying in bed, etc. If the recipient is laying in bed, even if the recipient is moving, the system could withhold the indication (the idea being that the movement is just the recipient rolling in bed for example). If the recipient is sitting in a chair, the system could withhold the indication until a movement indicative of the recipient standing up or otherwise significantly shifting his or her position within the seat occurs. Conversely, if the recipient is moving while standing for example, the indication would be immediate or otherwise as quickly as possible.

In an embodiment, the system is configured to provide an indication that the system is not activated within (inclusive) XYZ or more seconds or any value or range of values therebetween in 0.05 seconds of the system entering into the state of non-activation. XYZ can be 0.1, 0.25, 0.5, 0.75, 1, 1.25, 1.5, 1.75, 2, 2.5, 3, 3.5, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 75, 80, 85, 90, 120, 150, 210, 350, 300, 350, 400, 500, 600, 700, 800, 900, 1000, 1250, 1500 or more seconds. In an embodiment, the recipient and/or caregiver can adjust the timing of the indication and/or the device can be adjusted to adjust the timing by a professional and potentially only by a professional (adjustment might require reprogramming for example, or uploading new settings-with respect to user/caregiver adjustment, this could be executed for example by the recipient or caregiver depressing a button on the BTE device in a given sequence or in a certain manner so as to adjust the timing and/or could be accomplished by inputting the timing into a smart device or a remote control unit that is in signal communication with the prostatic component or the body worn component of the medical device. In some embodiments, a special GUI menu is provided on the display screen of the handheld device that allows the recipient to input the desired timing.

Note also that the above noted timings can be for the timing of the indication that the system is active and/or is not active. With respect to the former, it is envisioned that the timing could be longer than those detailed above, or at least longer than the timing for indication that the system is not activated (it is envisioned that the scenario where the system is not activated would be one in which the recipient would want to know sooner than the other scenarios). In any event, regardless of the scenario, in at least some exemplary embodiments, the above noted timings can be lengthened in some embodiments by 1.25, 1.5, 1.75, 2, 2.25, 2.5, 2.75, 3, 4, 5, 6, 7, 8, 9 or 10 times or any value or range of values therebetween in 0.05 increments.

Note also that embodiments include a recipient or third party check arrangement where the recipient or other caregiver queries the system as to whether the system is and/or is not activated. By way of example only and not by way of limitation, the recipient could depress a button on the BTE device for example, where the system is configured to interpret the depression of that button (or how the button was depressed-if the button was depressed three times in a row for example within two seconds, the system could interpret this as a query for the activation status, whereas if the button was depressed only twice within two seconds, the system could interpret this as a desire to adjust another functionality of the system by way of example-this is a long-winded way of saying that the teachings detailed herein can be utilized with single input device components or limited input device components) as a query from the recipient as to the status or operational mode, etc., of the system. Thus, in an exemplary scenario, the indication is only given in response to an affirmative action by the recipient that constitutes a query by the recipient as to if the system is activated and/or if the system is not activated.

Still, embodiments can include the arrangement where the system evaluates the activation state of the system, or at least the activation state of the portion that provides the stimulation or otherwise manages a bodily function of the recipient or otherwise manages a medical feature of the recipient, and provides an indication to the recipient or user or a third-party when the system is in a non-activation state. It is envisioned that regardless of the other features, a recipient may like the feature that the indication is provided when the system is not activated.

Embodiments include a non-transitory computer-readable media having recorded thereon, a computer program for executing at least a portion of a method, the computer program including code for automatically determining whether a system is functioning and/or is not functioning and/or how well the system is functioning and/or if the system is capable of functioning and/or how well the system will function, wherein the system is a sensory management and/or sensory stimulation system. Note that in some other embodiments, this functionality can instead be obtained by or in addition to this be obtained by a device or system configured to do such. In an embodiment, the code is configured to implement a checksum of certain data of the system or otherwise evaluate electrical currents and/or voltages of the system at certain nodes or otherwise utilitarian points in the overall circuitry of the system. The code can include logic to determine the level of functionality of the system based on such. Accordingly, in an embodiment, the code is code for an algorithm that receives for example, data indicative of the voltage at a location in the circuit over a certain period of time and/or at discrete periods of time, and then determines whether or not that voltage is present for a sufficient period of time and/or has a magnitude that is sufficient for the device to be functioning (at least at a certain level). By way of example only and not by way of limitation, the algorithm can average the voltage readings over a period of time, such as one or five or 10 seconds, and if the average (mean, median and/or mode) value of the voltage falls below a threshold, the algorithm includes logic to determine that the system is not functioning at a desired level. Additional implementations by way of example will be described below.

In an exemplary embodiment, the code for automatically determining whether the system is functioning and/or is not functioning and/or how well the system is functioning and/or if the system is capable of functioning and/or how well the system will function is code that performs a functionality check as distinguished from whether the system is activated and/or deactivated. By way of analogy, many central air systems and houses include electrostatic air filters. An electric charge is utilized to attract dust particles and adhere such to the filter. However, it is often difficult to ascertain whether or not the electrostatic system is functioning or otherwise how well it is functioning. The system could be on, but the electrostatic system of the air handling system may be in a state where the electrostatic feature is not performing to attract and/or adhere the dust particles to the filter (the basic filter is capturing the dust in the manner that would be the case without the electrostatic feature), or otherwise is performing but at a level that is meaningfully and even substantially below the intended operational level thereof. One may not be able to tell whether or not the electrostatic system is functioning by visual inspection. Conversely, measurements of current and/or voltage and/or even an evaluation of the static field(s) created by the system could provide an indication whether or not the system is functioning or how well the system is functioning. In a similar vein, here, there can be code that performs a functionality check. In an embodiment, this is executed while the system is turned on/the codes herein are executable while the system is turned on.

Conversely, the code could be implemented while the system is turned off. In this regard, a system that is off can correspond to a system that is in an inactive state and for all intents and purposes to any outside observer or to the recipient the system is off (e.g., the simulator portion is deactivated/will not stimulate for example, but consider test stimulation below). Indeed, in such an arrangement, the system would not be capable of providing stimulation or sensory management, etc., or one or more or all of the functionalities detailed herein associated with the medical devices'interface with the recipient or user, when in the off state. However, the system can still be capable of operating at a base level to perform diagnostic features or otherwise to execute at least some of the features associated with determining whether the system is capable of functioning and/or how well the system will function, etc. And note that in some embodiments, some form of output can be provided by the system, even though the system is “off.” The output could be some threshold level output or otherwise can be output of a limited duration that the recipient does not realize that the stimulation is present, or otherwise that the vast majority of recipients of the device or system will likely not realize that the stimulation is present, or even if they do, the effect their one will be de minimis. Accordingly, briefly jumping ahead, in an exemplary embodiment where efficacy of the medical device is evaluated as will be further detailed below, the efficacy evaluation is based on stimulation provided to the tissue of the recipient when the device was turned off and/or turned on.

Embodiment include utilizing the code for providing the indication with the code detailed above for automatically obtaining data based on a changeable environment of an impaired human, such as a balance impaired human and/or a movement-impaired human and/or a vestibular function-impaired human and code for controlling, at least partially, input to the brain of the human from a vestibula system of the human based on the obtained data. In some such embodiments, the code for automatically determining is code that makes the determination based on how the input to the brain is controlled (where, for example, the control can be halted, and thus the system is not functioning, or where the system is limited in its function, and thus the system is not operating at its full functionality). In an embodiment, as noted above, controlling at least partially input to the brain includes halting artificial stimulation of the vestibular system of the human and thus the indication would be that the system is not functioning for example. In an embodiment, there is code for automatically evaluating the obtained data and determining that a light level is at and/or below a threshold level, wherein the action of controlling at least partially input to the brain of the human including halting stimulation based on the determination.

In view of the preceding paragraph, it can be seen again that embodiments associated with controlling input to the brain of the human from a vestibula system of the human based on data pertaining to a changeable environment of an impaired human (balance impaired and/or movement impaired and/or vestibular function impaired, etc.) can be combined with the indication teachings detailed herein, consistent with the above-noted disclosure that any of these features can be combined with one another so as to provide the recipient and indication that the system is operating or not operating in accordance with the environmentally based controls.

In view of this, it can be seen that there are methods of providing an indication to the recipient or user or to a third-party that there exists, for example, a problem with the system or that the system will not function or otherwise will function in a certain manner that might be different than that desired or expected. The indication of this to the user recipient can be utilized by the user recipient to adjust his or her actions based on the knowledge of whether the system is functioning or will function or how the system is functioning or will function and/or adjust the medical device system. By rough analogy, in the case of a system that is a balance system for example, the system could be functioning at a level that will enable the recipient to walk around a house including walking up or down stairs at a desired level of safety, but would not be functioning at a level that would enable the recipient to drive a car on a winding road at rush hour, or at least would provide the recipient with notice that he or she should take more care than that which would otherwise be the case.

Briefly, we note that a functioning system is different from a system that is activated. As noted above, an activated system can be a system that is on but that does not mean that the system is in the state where the system can provide output and/or meaningful/utilitarian output. Conversely, a system that is functioning is a system that is providing some form of output for example, and typically utilitarian output. This requires at least in most conceivable embodiments that the system is on and otherwise activated. But functioning means working at some level. But note that there are levels of functionality. By way of example only and not by way of limitation, a system where the battery level is starting to run low may not function at a level corresponding to that which would be the case if the system had a fully charged battery. By way of example only and not by way of limitation, certain subroutines or functions could be curtailed owing to the low battery state. By way of example only and not by way of limitation, it could be that the system automatically curtails a current output of one or more electrodes and instead lengthens the amount of time that the current is applied. Indeed, the level of current output could simply be curtailed where all other things are held the same. The point is that there are different levels of functionality. In this regard, features associated with how well the system is functioning can include that the system will only provide certain levels of stimulation/management relative to other levels.

In an exemplary embodiment, there is also code for providing an indication that the system is functioning and/or is not functioning and/or how well the system is functioning. By way of example only and not by way of limitation, the code can be the code that is utilized to present the GUI interface of a smart phone as modified to have the specific display to present the indication. In an exemplary embodiment, the code can be code for allowing current to flow to a red colored LED, the illumination thereof indicating that the system is not functioning. In this regard, the code can be a code that evaluates a signal from the code that makes the determination of the functional level for example (output from a detector, which output could be digital, which output represents the presence or status of that signal), and upon failure to receive the signal, because the code that makes the determination has determined that the system is not functioning at the given level at issue, and thus stops outputting the signal to the algorithm for providing the indication, the code for providing the indication, when it performs a checksum or otherwise evaluates the current level of the signal, or the voltage of the signal, or more accurately, that the current and/or voltage is zero or de minimis, or otherwise that the output of the detector has a binary character string that corresponds to a lack of functioning of the system, provides the indication on the GUI that the medical device is not functioning and/or is not functioning at a desired level. The reverse can be true of course where a signal is received or otherwise the binary character indicates functionality, or at least functionality at a desired level, and in the code controls the GUI to provide an indication that the system is functioning at least in a desirable manner. This indication could be a graphic or a text string indicating that the system is functioning in a desired manner, or a text string indicating that the system is not functioning in a desired manner or not functioning at all for that matter, etc. The graphic could be a simple graphic such as a thumbs up or thumbs down image presented on the screen of the smartphone.

Consistent with the above, the code for automatically determining includes code to perform a self-assessment of the system. This self assessment can be executed in accordance with the teachings just noted above. Again, this can be executed by monitoring voltage and/or current of one or more parts of the system. In an embodiment, the code is code for monitoring the voltage of a battery or a power storage device or multiple power storage devices of the system. Again, in an exemplary embodiment, a voltage detector can be located in series with the output of a battery, and this detector can output a digital signal indicative of the voltage or at least a feature of the voltage, and is a digital signal that can be evaluated by the code, such as by comparing the data represented by the digital signal to a lookup table for certain values associated with the voltage, and if the lookup table indicates that for such value, the voltages at a level that is indicative of the system not functioning as desired or otherwise in a utilitarian manner, the code can make the determination in an automated manner. Again, other types of arrangements can be utilized where, for example, a detector is provided at other locations on the circuit, which detector monitors the voltage of the current etc., and outputs a digital signal which digital signal is evaluated by the code, such as based on a threshold technique or a lookup table technique.

In an embodiment, the code for automatically determining includes code to evaluate a feature indicative of an output of the system and determining that the system is and/or is not functioning and/or how well the system is functioning based on the evaluation of the feature. In an embodiment, this feature can be by way of example, the voltage of the electrode(s) and/or the current provided to the electrode(s). In an embodiment, voltage of the electrodes and/or the current supplied thereto can be measured utilizing standard techniques for measuring such with respect to cochlear implant technologies. In an embodiment, there is a current detector that detects the current supplied to the electrodes. The current detector outputs a digital signal which is evaluated by the software such as by way of a lookup table to compare the output to date of lookup table to determine whether or not the current is indicative of a functioning system or a system that is not functioning and/or to determine how well the system is functioning. In an embodiment, there can be another sensor that is separate from the output that evaluates the internal physiological features of the recipient to determine whether or not the system is functioning. Corollary to this is that this can be used to determine whether or not the system is providing efficacy and/or the level thereof. More on this below. In an embodiment, the feature indicative of the output could be the length of an electrical pulse that is output or the length of time that a voltage difference is present relative to other times, etc. In an embodiment, latent variables can be utilized as a basis, where a sensor can detect these latent variables and provide digital output to the software, where the output be compared to lookup tables for example. Any device, system, and/or method for capturing or otherwise ascertaining a feature indicative of the output of the system and/or for evaluating a feature indicative of the output of the system can be utilized in at least some exemplary embodiments providing that the art enables such, unless otherwise noted. Note further that more than one features that are indicative of the output of the system can be utilized, this is a failsafe method or a redundancy method while alternatively, the features can be compared to each other, where if these features establish a first set of features, the determination can be made that the system is functioning and if these features establish a second set of features, the determination can be made that the system is not functioning and/or that the sets of features can be utilized to determine how well the system is functioning and there can be code for such in some embodiments.

Note that features indicative of the output are different than whether or not a component is operating. Simply because an engine is running does not mean that the engine is propelling a vehicle, such as by way of example if the transmission of the vehicle is broken. To be clear, embodiments also include evaluating the operational aspects of the components of the system. Those could be related to output. But simply because a component is not working and thus there is no output does not mean that evaluation of that component corresponds to a per se evaluation of a feature indicative of the output.

In an embodiment, the code for providing an indication is code for providing a warning that the system is not functioning and/or will not function and/or is functioning at and/or below a specific level and/or will only function at and/or below a specific level. And note that “level” is used here loosely. That does not require, for example, a level of 77%, although such can be included in the level. Put another way, the level can be a qualitative and/or qualitative level. Any indication that will provide utilitarian value to the recipient or user or third party that is indicative of the specific level can be utilized in some embodiments providing that the art enables such, unless otherwise noted.

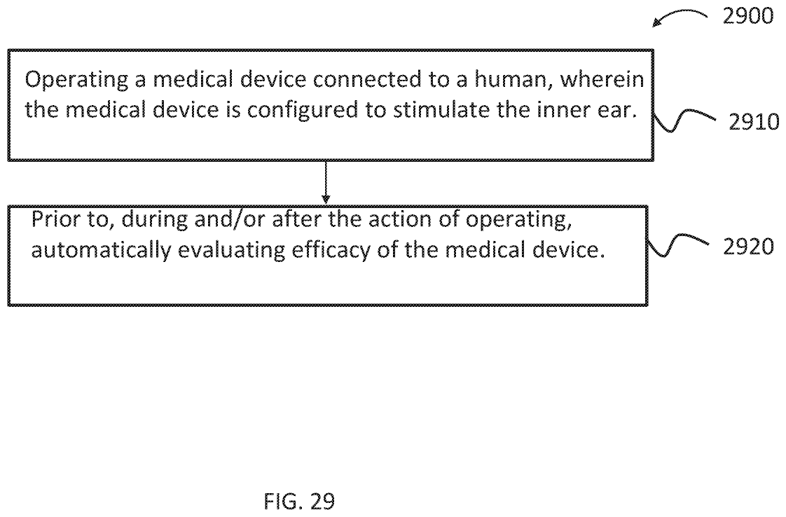

Embodiments include methods. FIG. 29 presents an exemplary flowchart for an exemplary method, method 2900, according to an exemplary embodiment. Method 2900 includes method action 2910, which includes the action of operating a medical device connected to a human, wherein the medical device is configured to stimulate the inner ear of the human. The stimulation could be direct stimulation or indirect stimulation. In an exemplary embodiment, the medical device can be a balance sensory medical device. In an exemplary embodiment, the device can be a tinnitus masking device. The device can be a cochlear implant. The device could also be a bone conduction device by way of example for that matter, because such stimulates the inner ear indirectly. The method also includes method action 2920, which includes the action of prior to, during and/or after the action of operating, automatically evaluating the efficacy of the medical device. Here, the efficacy could be in the context of whether the medical device is turned off, in which case it would have no efficacy. On the other extreme, the efficacy could be how well the medical device is functioning according to its intended purpose. By way of example only, in the example where the medical device is a balance sensory medical device, the efficacy could be in terms of how well the medical device is helping the recipient of the medical device to maintain balance. This can also be how well the medical device will help the recipient to maintain balance. This can be completely subjective to the recipient based on empirical data collected by the medical device while the medical device is connected to the human. The efficacy of the medical device can be evaluated based on feedback from the recipient by way of example. The medical device can be configured to receive this feedback and automatically evaluate such. The efficacy of the medical device can be based on the voltage difference between electrodes. By way of example, if the voltage difference is very high, this could be indicative of high impedance between the electrodes, indicating that the efficacy will be low. This is contrasted to, for example, evaluating the current level provided to electrodes to achieve a certain outcome and evaluating whether or not that is “a lot” of current relative to that which otherwise should be the case. That would be evaluating the efficacy of the medical device. If the medical device is capable of operating in an efficacious manner even though higher current levels are required, that is merely an inefficient device. If however the higher current levels were indicative of the device not having a level of efficacy relative to another level, that would be different and that would thus be automatically evaluating the efficacy of the medical device.

Still, embodiments can be focused on utilizing the state of the system in which the medical device is a part as a basis for evaluating the efficacy, off status, inactive status, low power status, reduced functionality status, etc. Here, this could be subjective or objective or both. In an exemplary embodiment, the evaluation of efficacy is based on whether the device is off. This would of course provide no efficacy. This action can be executed by a device separate from the medical device. This would thus not require the device to be able to operate to evaluate efficacy while the devices turned off in the normal manner that would be understood as being off. That is, the device would not require some form of routine that operates even though the device is turned off for all intents and purposes vis-à-vis the operational features desired by the user (e.g., to provide simulation for balance purposes). By way of example only and not by way of limitation, the portable handheld device that is part of the system, such as a smart phone, could be utilized to execute the action of automatically evaluating the efficacy of the medical device. Still, embodiments include methods where the action of evaluating is executed by the medical device while the medical device is off. Still, embodiments include scenarios where the action of automatically evaluating is executed while the device/system is on and the recipient would otherwise believe that the device is operating in an efficacious manner. And that is a utilitarian feature—the determination that there exists something anomalous or otherwise not what one would expect or want when the recipient would not necessarily realize that such is the case.

Consistent with the teachings herein, in an embodiment, the medical device is a balance sensory medical device and the efficacy is based on how well the device is performing to improve balance of the human. This would be based on subjective data specific to the human. In an exemplary embodiment, there can be a system that can be configured to automatically evaluate the movements of the human for example, and compare those movements to a baseline, which could be stored in a memory. Based on this comparison, an evaluation can be made as to how well the device is performing to improve the balance of the human. In an exemplary embodiment, there can be one or more gyroscopes or inertia sensors, etc., that could capture data relating to the movements of the human (or lack of movement by the human, indicating that the human may be experiencing dizziness) and provide an output that is evaluated by the code, where the code is configured to evaluate such data by comparing the data to for example lookup tables. In an alternate embodiment, the system could receive feedback from the recipient, such as for example he or she feels dizzy, etc., and that could be the basis for the evaluation. This input can be verbal and/or can be input into the system by the smart device touch screen or by input directly into the BTE device of the balance medical device by way of example (tapping the device a number of times indicating the level of dizziness for example).

Still, embodiments associated with feedback from the recipient based on the recipient's observations defeat the concept of a system that checks itself or otherwise a system where there is an automated evaluation of the efficacy or the functionality, etc., of the system, so as to provide an indication that serves as a warning or otherwise notification to the recipient or user that the device is not functioning in a manner that he or she may believe it is functioning or otherwise should be functioning. That is, embodiments can have utilitarian value with respect to notifying the recipient that he or she should not rely on the medical device to the full extent that he or she might otherwise rely on the medical device, where the recipient could, in some implementations, have little to no reason to believe that this would be the case. This is somewhat analogous to the electrostatic air cleaner analogy detailed above. It is an indication that there is a problem or at least there is scenario that is unfolding that is not readily apparent or otherwise may not be readily apparent to the recipient. Accordingly, embodiments include devices, systems, and/or methods of performing the evaluations detailed herein without at least direct input from the recipient and/or without input from the recipient that is based on an evaluation by the recipient or otherwise a judgment of the recipient. Embodiments include devices, systems and/or methods of performing the evaluations without the recipient needing to take any action other than at most utilizing the medical device as would normally be utilized.

As noted above, the action of automatically evaluating the efficacy of the medical device can be executed while the device is operating or before or after the device is operated, or all three. In an exemplary embodiment, the action of evaluating is executed within and/or before (including at least before) and/or after (including at least after) any of the above-noted time periods, which are not repeated here but incorporated by reference for purposes of textual economy. Thus, for example, the action of automatically evaluating the efficacy could be executed 120 seconds or at least 120 seconds before operating the medical device. This can have utilitarian value with respect to giving the recipient of the balance sensory prosthesis for example time to change or adjust his or her intended actions depending on how efficaciously the medical device is going to operate or otherwise is operating or has been operating. For example, if the medical device is operating at a high level of efficacy, the recipient may determine to engage in activities that require a high level of balance or otherwise where if the recipient loses his or her balance, the results could be more drastic than other activities with which the recipient will participate. That is, this can enable risk management at a superior level relative to that which might otherwise be the case. (It is not reasonable to have a recipient of a balance sensory prosthesis to avoid walking across a hard asphalt parking lot at all times. That is not risk management. That is risk avoidance. Determining when or whether the recipient should walk across a hard asphalt parking lot is risk management.) In any event, consistent with the teachings detailed above, embodiments include evaluating the efficacy of the medical device while the medical device is being operated. But also consistent with the teachings detailed above, the action of automatically evaluating the efficacy of the medical device could also be implemented when the devices cease to be operational, and this could be a very utilitarian time to evaluate the efficacy, because this can be utilized as a basis to convey to the recipient that the efficacy of the medical device is low (which includes no efficacy). And embodiments include executing the efficacy evaluation within a short time of the cessation of operation.

Note also that any disclosure herein of a temporal trigger for the device not being activated or not operating or being off corresponds to an alternate disclosure of the device changing a level of efficacy or state, etc. Any change of state/new state that warrants an indication to the recipient or a third party is applicable to any of the temporal triggers detailed herein unless otherwise noted, all in the interests of textual economy. With respect to arrangements that are agnostic to whether the device is turned on or turned off, in an exemplary embodiment, the efficacy is based on whether the device is providing tissue stimulation to the human regardless of or turned off. Again, this could be providing stimulation to the recipients at a sub threshold level while the device is turned off, which stimulation is provided for the diagnostic purposes detailed herein.

Consistent with the disclosure above regarding the utilization of the indication features and other features in combination with the varied ambient environment embodiments, in an exemplary embodiment of an exemplary method, the action of automatically evaluating the efficacy is based on the medical device being operated based on the varied ambient environment. By way of example only and not by way of limitation, in an embodiment where the medical device is a balance sensory medical device, the method can include obtaining data based on an ambient environment of a balance-impaired human. The method can further include experiencing a variation of the ambient environment, such as when the lights go off in a living room for example. The method further includes limiting operation of the balance sensory medical device connected to the human based on the varied ambient environment. In an exemplary embodiment, the action of automatically evaluating the efficacy of the medical device in method action 2920 is based on the medical device being limited in operation based on the varied ambient environment. Conversely, in an exemplary embodiment, the method can further include experiencing a variation of the ambient environment where lighting in a room is increased to a level that renders the balance sensory medical device utilitarian, and thus the balance sensory medical device is activated from a deactivated state by way of example. Here, the method includes limiting operation of the balance sensory medical device based on the varied ambient environment, or otherwise activating the balance sensory medical device, such as from a deactivated state or otherwise increasing the level of functionality of the medical device.

Consistent with the teachings detailed above, embodiments can include providing indication to the recipient of the medical device or ⅓ party, etc., of the limitation and/or the removal of limitations.

In an exemplary embodiment, the system and/or device is configured to perform, and the methods of performing include, a check on the activation and/or functionality and/or efficacy of the system and/or device every and/or within XYZ seconds or any value or range of values therebetween in 0.05 second increments during a 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or 100 minute period, or any value or range of values therebetween in 0.1 minute increments, and the timing between performances need not be the same during those time periods (e.g., during a 30 minute period, the device could perform a check every 5 seconds for 18 minutes and then every 3 seconds for 12 minutes). The utilized because the recipient is engaging in an activity where even a temporary drop in efficacy for a short period of time could be life threatening vs. that which would be the case for other activities. Accordingly, embodiments include methods where the action of automatically evaluating is executed periodically (even or different timings between evaluation) by the medical device/system while the device is connected to the human and/or while the medical device/system is in a state where the recipient thereof would believe that the device is functioning normally/system is functioning normally or otherwise functioning in a manner as desired. The method further includes periodically automatically providing an indication to the human that the device is operating in an efficacious manner based on the results of the automatically evaluating. The periods need not coincide or be temporally linked in a consistent manner, but can be as well. Returning back to embodiments where the medical device is a balance sensory medical device, the action of automatically evaluating can be executed periodically by the balance sensory medical device while the device is connected to the human and/or while the medical device/system is in a state where the recipient thereof would believe that the device is functioning normally/system is functioning normally or otherwise functioning in a manner as desired and the method further comprises automatically providing an indication to the human that the device is operating in a less than efficacious manner based on the results of the automatically evaluating.

For purposes of textual economy, the disclosure herein often refers to one feature without explicitly referring to another feature. By way of example only and not by way of limitation, some of the disclosure herein refers to activation while other portions of the disclosure refer to functionality while other portions of the disclosure referred to efficacy. For purposes of textual economy, it is noted that any disclosure of a feature related to activation and/or non-activation corresponds to an alternate disclosure related to functionality and/or non-functionality and/or various levels thereof and/or efficacy or non-efficacy or variation of levels thereof provided that the art enables such unless otherwise specifically noted. Any feature of embodiment can be combined with any other embodiment and/or any feature of any embodiment can be explicitly excluded from use with any feature of any other embodiment unless otherwise noted providing that the art enables such.

And while embodiments have focused on detailed arrangements associated with evaluating features associated with functionality and/or activation and/or efficacy, embodiments also include basic implementations where the system provides an indication to the recipient or user thereof of the given status of the system and/or a sub-system thereof, such as the first subsystem, including whether the system and/or a portion of that system, such as a stimulator device for a prosthesis for example, is on and/or off and/or whether such is functional and/or not functional at a base level (e.g., the system can be turned on, but the battery is so weak that it cannot stimulate the vestibular system sufficiently or consistently to achieve the desired efficacy). By way of example only and not by way of limitation, a portable handheld device such as a smart phone can provide an indication that the balance sensory medical device is turned off or otherwise is not on or otherwise is not functioning and/or vis-a-versa. In an embodiment, the system is configured to detect the voltage and/or current that is provided to or can be provided to the stimulator unit of the given prostheses in question and output a signal, such as a digital signal, that can be read by code of the system and/or evaluated by a processor of the system, etc., by comparing the voltage and/or current to that of a lookup table in determining whether or not the system can function or is functioning, etc. In an embodiment, the system is configured to detect the on off state of the stimulator, which can correspond to evaluating the state of the switch that turns the system on and/or off, etc., and this detection can be the basis for determining the state of the system.

To reiterate, in view of the above, there can be utilitarian value with respect to utilizing the teachings detailed herein so as to provide an indication to a recipient of the system and/or a caregiver, that the system is functioning and/or is not functioning etc. This can reduce the likelihood of the scenario where the recipient or a caregiver has false confidence in the system or otherwise the results of the system relative to that which would otherwise be the case. In a straightforward scenario, a recipient who is suffering from balance disorders and is utilizing a balance sensory medical device will achieve utilitarian value with respect to knowing the state of the system before and/or during activities that require balance, or at least where the results of lacking balance could be bad relative to other scenarios, such as driving versus lying in bed or walking across a hard asphalt parking lot versus walking across a manicured lawn with a foot or two of topsoil, etc.

As noted above, embodiments include methods that include automatically evaluating the efficacy of the medical device. This can be done utilizing logic circuitry and/or software that analyzes certain features of the system of which the medical device is a part. These features can be latent variables associated with the efficacy of the medical device, for example, or can be more directly associated therewith. The component that executes the action of automatically evaluating is more than a mere on-off indicator. Indeed, simply indicating whether or not the medical device is on or off is not evaluation. That is the conveyance of information. Conversely, by way of example, a logic circuit that automatically evaluates whether or not the system is in an activation state to energize the electrodes, whether or not specially designed and implemented to execute the teachings herein, can correspond to a circuit for implementing some of the method actions detailed herein. If that circuit also includes output that can control for example the activation/deactivation of an LED, which LED indicates whether or not the system is on for example, those features are ancillary to the logic circuit that automatically evaluates whether or not the system is operating, etc. Note further that componentry that is utilized to execute some of the method actions detailed herein can utilize existing circuitry in the system that for example provides for the control of the aforementioned LED. By way of example only and not by way of limitation, if a medical device is configured to provide electrical current to an LED to indicate that the device is turned on, some embodiments include monitoring for that electrical current and utilizing the detection thereof or the absence of detection thereof as an indicator when executing at least some of the exemplary method actions. That said, systems can include more sophisticated arrangements where the circuitry that is utilized to controllably generate the electrical current is utilized to also as a basis implement at least some of the method actions, or at least output from such circuitry is utilized as a basis to make determinations with respect to some of the method actions detailed herein. To be clear, mere on/off indicators/use thereof does not constitute evaluating efficacy or determining whether a system is functioning or not or how well the system is functioning/whether the system is capable of functioning, etc.

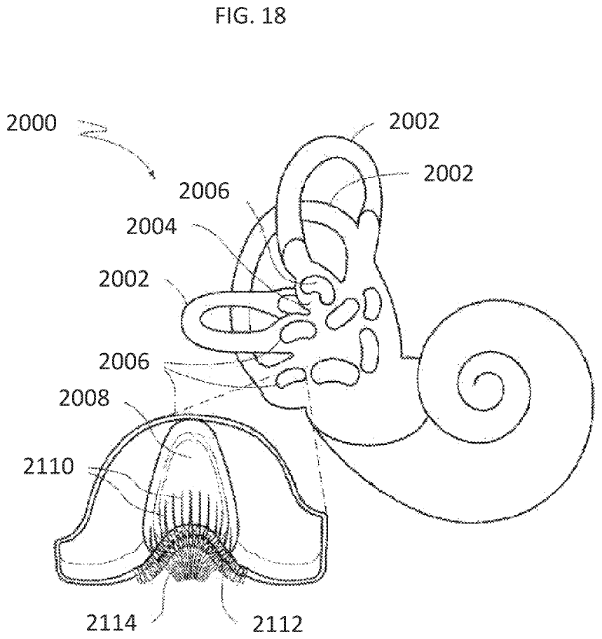

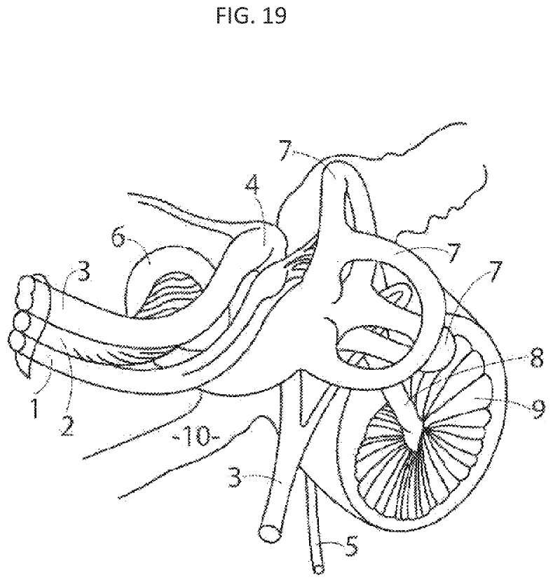

Some additional details of vestibular stimulation will now be described. FIG. 18 is a general schematic illustration of the anatomical structures of the vestibular system and FIG. 19 is a more detailed illustration of the semicircular canals and related structures. With reference to FIG. 18, there is a vestibular system 2000. The three semi-circular canals 2002 are shown, each being arranged more or less orthogonal to each other. Each canal is filled with endolymph fluid, and upon rotation of the head with a component of motion in the appropriate direction, fluid is caused to move within the canal. At the base of each canal is the ampulla 2004 and the related crista 2006. Within the crista 2006 is the cupula 2008 which contains hair bundles 2110 connected to hair cells 2112, and in turn to nerve fibres 2114. When the fluid moves, the hair cells 2112 are stimulated, and produce a corresponding neural signal. With reference to FIG. 19, there is an illustration in more detail the location and orientation of the vestibular labyrinth relative to cranial nerves VII and VIII and selected structures of the inner and middle ear. Illustrated are Nervus vestibularis 1, Nervus cochlearis 2, Nervus intermediofacialis 3, Ganglion geniculi 4, Chorda tympani 5, Cochlea 6, semicircular canals 7, Malleus 8, tympani 9, and ear canal 10. The illustrative embodiment which will be described can be used in a relatively simple, constant stimulation system. This is intended to be operable by a user when they determine that they have symptoms indicating the onset of an attack, or alternatively in a preventative mode, in which the device is operated to prevent the onset of an attack. In other embodiments, the system could be implemented in a manner which is connected to a monitor that automatically enables/disables stimulation per the above and/or adjusts such per the above. Embodiments can include direct electrical stimulation of the vestibular system by implanting an electrode array atraumatically within one or more semicircular canals.

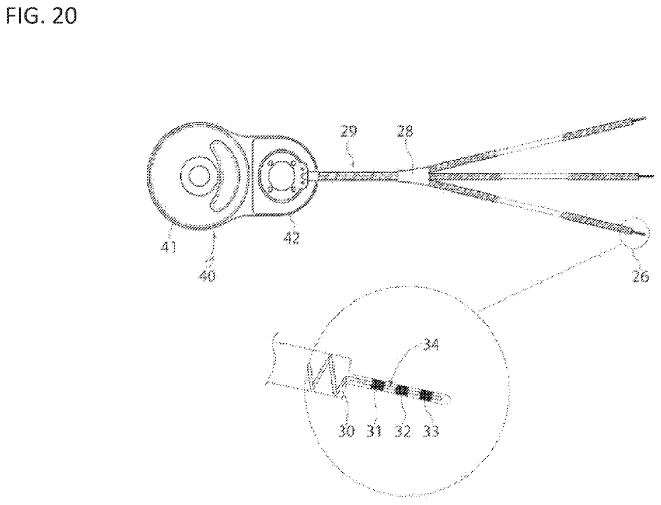

An embodiment of a vestibular stimulation device, such as those above and/or presented in FIG. 20 (more on this in a moment), can include an external processor (or an implanted processor), a transmit coil (transcutaneous link) and implant and an electrode array. The stimulation device 40, and associated external power supply/stimulation controller, can be a modified conventional cochlear implant stimulator device, with a customized electrode array.

FIG. 20 shows an enlarged view of the array, showing the electrodes 31, 32, 33, with a stiffener 34.

Once implanted, the electrodes of the electrode array (although embodiments can use a single electrode on each branch) receive stimulation signals from the stimulator unit. The stimulator unit is typically electrically connected to the electrode array by an electrical lead. The stimulator unit is positioned within a housing that is implantable within the patient, and is typically implanted within a recess in the bone behind the ear posterior to the mastoid. When implanted, the housing can include, in addition to the stimulator unit, a receiver unit adapted to receive signals from a controller. The controller is, in this example, mounted external to the body behind the pinna of the patient such that signals are transmitted transcutaneously through the skin of the patient.

The signals travel from the controller to the receiver unit and vice versa. The receiver unit includes a receiver antenna, such as an antenna coil, adapted to receive radio frequency (RF) signals from a corresponding transmitter antenna, such as an antenna coil, worn externally of the body. The radio frequency signals may comprise frequency modulated (FM) signals, but could alternatively be modulated in any suitable way, using, amplitude, frequency or phase, using either analog or digital techniques. In general, the modulation can be chosen in order to maximize both the data and power efficiency of the link. It should be appreciated that the receiver antenna may also transmit signals, and that the transmitter antenna may receive such signals. The transmitter antenna coil is preferably held in position adjacent the implanted location of the receiver antenna coil by way of respective attractive magnets (not shown) mounted centrally in, or at some other position relative to, the coils.

The external controller in this example includes a processor (not shown, but can correspond to the processor(s) noted above) adapted to encode a suitable stimulation signal, for example in response to any one or more of the triggers detailed herein. Such a signal can include data defining, for example, the mode of stimulation, current level, and which electrodes are to be stimulated. As the implant can use three separate electrode arrays, the stimulation may occur on more than one array simultaneously, or alternatively, sequentially. The encoded sequence is transferred to the implanted receiver/stimulator unit using the transmitter and receiver antennae. The implanted receiver/stimulator unit demodulates the signals and allocates the electrical pulses to the appropriate electrode. The external controller may further include a power supply (not shown). The power supply may comprise one or more rechargeable batteries. The transmitter and receiver antennae are used to provide power via transcutaneous induction to the implanted receiver/stimulator unit and the electrode array.

Embodiments of the implants can be configured to deliver both monopolar and bipolar stimulation or one or the other. Bipolar stimulation occurs when a current flows from one electrode to another electrode of the same array, that is, in the same canal. Monopolar stimulation occurs when current flows between an electrode within the canal and an electrode external to the canal, for example a separate implanted electrode external to the canal. Depending on the stimulation current required to elicit a response bipolar may be advantageous in minimizing interaction with adjacent semicircular canals. At least two channels, typically one intracanal and one inter canal, are also required for Neural Response Telemetry which has been shown to be important during surgery for electrode placement.

When there is no movement, the normal vestibular system generates constant regular activity, i.e., the neurons in the semicircular canal fire at a constant rate. The objective of stimulation, in some embodiments, can be to simulate this constant firing through delivery of electrically evoked afferent activity. This may, according to some embodiments, be unmodulated. However it is contemplated that other implementations may be modulated, for example in frequency or amplitude, in order to provide more complex user percepts. The electrical stimuli that can be used can be a lower complexity and at lower rate pulse trains than for auditory stimulation. For example, the electrical stimuli may be provided as biphasic pulses at 100-200 Hz, 400 μs phase width, 8 μs phase gap and currents of between 20-100 uA. These figures are indicative only, and implementations may use other parameters.

In an embodiment, the electrode array is intended into each of the semi-circular canals whilst preserving any residual vestibular function. This is achieved using a suitable dimension, for example for a circular array, a diameter less than 150 microns. Other specific characteristics, relating to length, a stopper to limit penetration, and stiffness assist in this objective, as will be explained further below.

Embodiments can instead be located on the outside of the canals, or both places.

The array(s) can be surgically placed to either one, two or all semicircular canals.

The illustrated arrangement of electrode array(s) can allow for the placement of one electrode array in one semicircular canal, with the remaining electrode arrays placed safely within the mastoidectomy cavity for possible future implantation in the remaining semicircular canals. In this case, only the implanted array is used for stimulation. The remaining electrode arrays could also be used for possible otolithic stimulation via implantation of the vestibule, possibly via a round or oval window approach, or via the common crus.

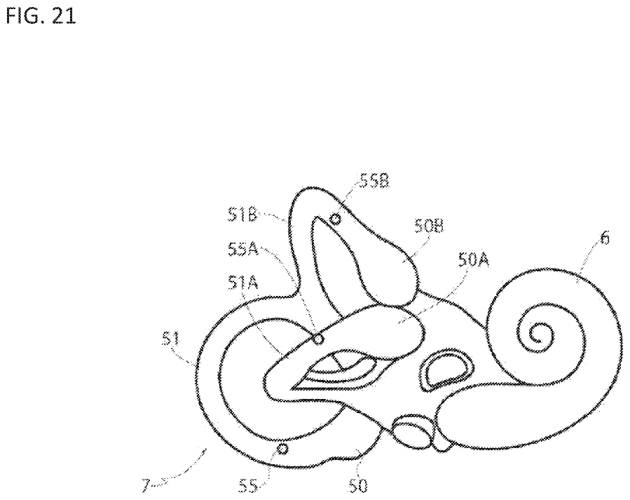

FIG. 21 illustrates suitable surgical openings 55, 55A, 55B in the posterior 51, superior 51B and lateral 51A semi-circular canal, through which the electrode array 26 may be implanted. In each case, the respective ampulla 50, 50A, 50B can be seen.

The array can be operatively placed within the labyrinth whilst preserving vestibular function/sensitivity, but providing robust electrical stimulation of the vestibular periphery.

Each array can have a sufficient number of electrodes to permit both monopolar and bipolar stimulation, as well as to provide sufficient redundancy in the event of individual electrode failure. A suitable reference electrode can also be provided as a return path for monopolar stimulation.

Some embodiments include implantation away from the ampulla of the lateral canal.

Embodiments can include electrode placement near the ampullae of the semicircular canals for activation of the vestibular system.

Embodiments include the utilization of machine learning algorithms or otherwise so-called artificial intelligence to implement at least some of the teachings detailed herein. In an exemplary embodiment, these machine learning algorithms can be implemented to evaluate the environment, such as the light level or the visual scene, etc., make a determination based on that evaluation, such as, for example, whether or not to suppress or inhibit the vestibular balance function in accordance with the teachings detailed above, by way of example only. Some brief examples of the implementations of machine learning technology will now be described, but it is also noted that in an exemplary embodiment, the analyses/decisions detailed herein can be executed by a microprocessor or a chip or otherwise electronic circuitry with logic circuits configured to analyze the data and make decisions thereon.

At least some exemplary embodiments according to the teachings detailed herein utilize advanced techniques to analyze the data obtained by the system/apparatus and/or used in the methods. An exemplary data processing technique is the so called deep neural network (DNN). At least some exemplary embodiments utilize a DNN (or any other advanced learning data processing technique) to process data, which processed data is utilized to evaluate the electrodes according to the teachings herein. At least some exemplary embodiments entail training data processing algorithms to process data to implement at least some of the exemplary methods herein. That is, some exemplary methods utilize learning algorithms or regimes or systems such as DNNs or any other system that can have utilitarian value where that would otherwise enable the teachings detailed herein to analyze the data.

Embodiments include utilizing a so-called “neural network” that can be a specific type of machine learning system. Any disclosure herein of the species “neural network” constitutes a disclosure of the genus of a “machine learning system.” While embodiments herein focus on the species of a neural network, it is noted that other embodiments can utilize other species of machine learning systems. Accordingly, any disclosure herein of a neural network constitutes a disclosure of any other species of machine learning system that can enable the teachings detailed herein and variations thereof. To be clear, at least some embodiments according to the teachings detailed herein are embodiments that have the ability to learn without being explicitly programmed. Accordingly, with respect to some embodiments, any disclosure herein of a device or system constitutes a disclosure of a device and/or system that has the ability to learn without being explicitly programmed, and any disclosure of a method constitutes actions that results in learning without being explicitly programmed for such.

Embodiments include method actions associated with processes to train DNNs so as to enable those DNNs to be utilized to execute at least some of the method actions detailed herein.

It is noted that in at least some exemplary embodiments, the DNN or the product from machine learning, etc., is utilized to achieve a given ability to evaluate/process the data detailed herein. In some instances, for purposes of linguistic economy, there will be disclosure of a device and/or a system that executes an action or the like, and in some instances structure that results in that action or enables the action to be executed. Any method action detailed herein or any functionality detailed herein or any structure that has functionality as disclosed herein corresponds to a disclosure in an alternate embodiment of a DNN or product from machine learning, etc., that when used, results in that functionality, unless otherwise noted or unless the art does not enable such.

Exemplary embodiments include utilizing a trained neural network to implement or otherwise execute at least one or more of the method actions detailed herein, and thus embodiments include a trained neural network configured to do so. Exemplary embodiments also utilize the knowledge of a trained neural network/the information obtained from the implementation of a trained neural network to implement or otherwise execute at least one or more of the method actions detailed herein, and accordingly, embodiments include devices, systems and/or methods that are configured to utilize such knowledge. In some embodiments, these devices can be processors and/or chips that are configured utilizing the knowledge. In some embodiments, the devices and systems herein include devices that include knowledge imprinted or otherwise taught to a neural network.

All the above said, in some embodiments, standard processors that are programmed in the traditional manner/that are not machine learning based and/or chips that are formatted in a traditional manner and include logic circuitry that are configured to execute at least some of the exemplary method actions detailed herein are utilized. Computers and computational devices that are programmed or otherwise configured to accept the data and/or retrieve the data and/or process the data or otherwise evaluate the data can be utilized to execute at least some of the method actions detailed herein. Note also that any reference to a method action herein that is implemented utilizing artificial intelligence and/or a neural network and/or machine learning corresponds to an alternate embodiment where the reference is to a functionality of a device. By way of example only and not by way of limitation, in an exemplary embodiment, if there is a disclosure herein of a medical device that is configured to evaluate the data relating to the ambient environment, and take some action based thereon, such corresponds to a disclosure of utilizing a product of machine learning to analyze that data, where the product of the machine learning can be a computer chip for example, which computer chip can be part of the medical device or the smart device/assistant device, or the home/work/infrastructure system, etc.

It is noted that any method action or functionality disclosed herein corresponds to a disclosure of a non-transitory computer readable medium that has program thereon a code for executing such method action providing that the art enables such. Still further, any method action or functionality disclosed herein where the art enables such corresponds to a disclosure of a code from a machine learning algorithm and/or a code of a machine learning algorithm for execution of such. Still, in an exemplary embodiment, the code need not necessarily be from a machine learning algorithm, and in some embodiments, the code is not from a machine learning algorithm or the like. That is, in some embodiments, the code results from traditional programming. Still, in this regard, the code can correspond to a trained neural network. That is, as will be detailed below, a neural network can be “fed” significant amounts (e.g., statistically significant amounts) of data corresponding to the input of a system and the output of the system (linked to the input), and trained, such that the system can be used with only input, to develop output (after the system is trained). This neural network used to accomplish this later task is a “trained neural network.” That said, in an alternate embodiment, the trained neural network can be utilized to provide (or extract therefrom) an algorithm that can be utilized separately from the trainable neural network. In one embodiment, there is a path of training that constitutes a machine learning algorithm starting off untrained, and then the machine learning algorithm is trained and “graduates,” or matures into a usable code-code of trained machine learning algorithm. With respect to another path, the code from a trained machine learning algorithm is the “offspring” of the trained machine learning algorithm (or some variant thereof, or predecessor thereof), which could be considered a mutant offspring or a clone thereof. That is, with respect to this second path, in at least some exemplary embodiments, the features of the machine learning algorithm that enabled the machine learning algorithm to learn may not be utilized in the practice some of the method actions, and thus are not present the ultimate system. Instead, only the resulting product of the learning is used.

And to be clear, in an exemplary embodiment, there are products of machine learning algorithms (e.g., the code from the trained machine learning algorithm) that are included in any one or more of the systems/subsystems detailed herein, that can be utilized to analyze any of the data obtained or otherwise available disclosed above that can be utilized or otherwise is utilized to evaluate the utilitarian value of any one or more of the implants detailed herein. This can be embodied in software code and/or in computer chip(s) that are included in the system(s).

An exemplary system includes an exemplary device/devices that can enable the teachings detailed herein, which in at least some embodiments can utilize automation. That is, an exemplary embodiment includes executing one or more or all of the methods detailed herein and variations thereof, at least in part, in an automated or semiautomated manner using any of the teachings herein. Conversely, embodiments include devices and/or systems and/or methods where automation is specifically prohibited, either by lack of enablement of an automated feature or the complete absence of such capability in the first instance.

It is further noted that any disclosure of a device and/or system detailed herein also corresponds to a disclosure of otherwise providing that device and/or system and/or utilizing that device and/or system.

It is also noted that any disclosure herein of any process of manufacturing other providing a device corresponds to a disclosure of a device and/or system that results there from. Is also noted that any disclosure herein of any device and/or system corresponds to a disclosure of a method of producing or otherwise providing or otherwise making such.

An exemplary system includes an exemplary device/devices that can enable the teachings detailed herein, which in at least some embodiments can utilize automation. That is, an exemplary embodiment includes executing one or more or all of the method actions detailed herein and variations thereof, at least in part, in an automated or semiautomated manner using any of the teachings herein.

It is further noted that any disclosure of a device and/or system detailed herein also corresponds to a disclosure of otherwise providing that device and/or system and/or utilizing that device and/or system.

It is also noted that any disclosure herein of any process of manufacturing other providing a device corresponds to a disclosure of a device and/or system that results there from. Is also noted that any disclosure herein of any device and/or system corresponds to a disclosure of a method of producing or otherwise providing or otherwise making such.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

Any function or method action detailed herein corresponds to a disclosure of doing so an automated or semi-automated manner.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A system, comprising:
a first subsystem configured to neurologically affect a human when activated; and
a second subsystem configured to obtain data based on an ambient environment of the system, wherein
the system is configured to control the first subsystem, at least in part, based on the obtained data, and
the system is a human balance medical system.

2. The system of claim 1, wherein:
the first subsystem is a vestibular neurological system stimulation device.

3. The system of claim 1, wherein:
the first subsystem is a therapeutic substance delivery subsystem.

4. The system of claim 1, wherein:
the second subsystem is a light capture subsystem.

5. (canceled)

6. The system of claim 1, wherein:
the system is configured to limit a level of effect of the first subsystem on the human based on the obtained data.

7. The system of claim 1, wherein:
the first subsystem is configured to varyingly suppress and/or inhibit sensory input to a brain of the human.

8-37.

38. A human balance medical system, comprising:
a neurological stimulator subsystem configured to influence neurological signals to a brain of a recipient of the human balance medical system to improve balance of the recipient; and
a power source, wherein
the neurological stimulatory subsystem is powered by the power source, and
the human balance medical system is a smart human balance medical system.

39. The system of claim 38, wherein:
the smart human balance medical system is configured to control the neurological stimulator subsystem based on input into the system indicative of an ambient environment of the system.

40. The system of claim 38, wherein:
the smart human balance medical system is configured to automatically adjust an environment of the system to improve efficacy of the neurological stimulator subsystem.

41. The system of claim 38, wherein:
the smart human balance medical system is configured to automatically adjust a functionality of the neurological stimulator subsystem based on ambient light and/or noise level.

42. The system of claim 38, wherein:
the smart human balance medical system is configured to automatically halt a functionality of the neurological stimulator subsystem based on an ambient light level and/or ambient noise level.

43. The system of claim 38, wherein:
the smart human balance medical system is configured to automatically check an ambient light level and automatically, based on the checked ambient light level, selectively:
(i) adjust a functionality of the neurological stimulator subsystem;
(ii) halt a functionality of the neurological stimulator subsystem; or
(iii) provide information and/or a recommendation to the recipient to improve balance of the recipient.

44. The system of claim 38, wherein:
the smart human balance medical system is configured to gather latent variable(s) that can impact recipient cognitive load and automatically, based on the gathered latent variable(s), selectively:
(i) adjust a functionality of the neurological stimulator subsystem;
(ii) halt a functionality of the neurological stimulator subsystem; or
(iii) provide information and/or a recommendation to the recipient to improve balance of the recipient.

45-47. (canceled)

48. A system, comprising:
a first subsystem configured to neurologically affect a human when activated; and
a second subsystem configured to provide an indication that the system is activated and/or not activated, wherein
the system is a sensory management and/or sensory stimulation system.

49. (canceled)

50. The system of claim 48, wherein:
the system is an epilepsy management system.

51. The system of claim 48, wherein:
the system is a tinnitus management system or a sleep apnea system.

52. (canceled)

53. The system of claim 48, wherein:
the second subsystem is configured to provide an indication that the system is off.

54-55. (canceled)

56. The system of claim 48, wherein:
the second subsystem is configured to provide the indication to a recipient of the system, which indication is visual.

57. (canceled)

58. The system of claim 48, wherein:
the second subsystem is configured to directly provide the indication to a recipient of the system from a body worn portion of the system.

59-61. (canceled)

62. The system of claim 48, further comprising a light sensor.

63-99. (canceled)
